(19) United States
(12) Patent Application Publication
(10) Pub. No.: US 2007/0078083 A1
(43) Pub. Date: Apr. 5, 2007

(54) MODULATION OF NEUORGENESIS BY HDac INHIBITION

(75) Inventors: Carrolee Barlow, Del Mar, CA (US); Todd A. Carter, San Diego, CA (US); Kym I. Lorrain, San Diego, CA (US); Jammieson C. Pires, San Diego, CA (US); Andrew Morse, San Diego, CA (US); Dana Gitnick, San Marcos, CA (US); Kai Treuner, San Diego, CA (US); Alejandro R. Dearie, Chula Vista, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: BrainCells, Inc., San Diego, CA (US)

(21) Appl. No.: 11/470,957

(22) Filed: Sep. 7, 2006

Related U.S. Application Data
(60) Provisional application No. 60/715,219, filed on Sep. 7, 2005.
(60) Provisional application No. 60/764,963, filed on Feb. 3, 2006.
(60) Provisional application No. 60/785,713, filed on Mar. 24, 2006.

(51) Int. Cl. A61K 38/12; A61K 31/19

(52) U.S. Cl. 514/9; 514/575

(57) ABSTRACT

The instant disclosure describes methods for treating diseases and conditions of the central and peripheral nervous system by stimulating or increasing neurogenesis. The disclosure includes compositions and methods based on an HDac inhibitory agent alone or in combination with another neurogenic agent to stimulate or activate the formation of new nerve cells.

### CROSS-REFERENCES TO RELATED APPLICATIONS

This application claims benefit of priority under 35 U.S.C. §119(e) from U.S. Provisional Patent Applications 60/715,219, filed Sep. 7, 2005; 60/764,963, filed Feb. 3, 2006; 60/785,713, filed Mar. 24, 2006; all three of which are hereby incorporated by reference as if fully set forth.

### FIELD OF THE DISCLOSURE

The instant disclosure relates to methods for treating diseases and conditions of the central and peripheral nervous system by stimulating or increasing neurogenesis via inhibition of histone deacetylase (HDac) activity, including via inhibition of the activity in combination with another neurogenic agent. The disclosure includes methods based on the application of a neurogenesis modulating agent having inhibitory activity against HDac activity to stimulate or activate the formation of new nerve cells.

### BACKGROUND OF THE DISCLOSURE

Neurogenesis is a vital process in the brains of animals and humans, whereby new nerve cells are continuously generated throughout the life span of the organism. The newly born cells are able to differentiate into functional cells of the central nervous system and integrate into existing neural circuits in the brain. Neurogenesis is known to persist throughout adulthood in two regions of the mammalian brain: the subventricular zone (SVZ) of the lateral ventricles and the dentate gyrus of the hippocampus. In these regions, multipotent neural progenitor cells (NPCs) continue to divide and give rise to new functional neurons and glial cells (for review Gage 2000). It has been shown that a variety of factors can stimulate adult hippocampal neurogenesis, e.g., adrenalectomy, voluntary exercise, enriched environment, hippocampus dependent learning and anti-depressants (Yehuda 1989, van Praag 1999, Brown J 2003, Gould 1999, Malberg 2000, Santarelli 2003). Other factors, such as adrenal hormones, stress, age and drugs of abuse negatively influence neurogenesis (Cameron 1994, McEwen 1999, Kuhn 1996, Eisch 2004).

In eukaryotic cells, nuclear DNA wraps around a protein core consisting of histones H2A, H2B, H3, and H4 to form chromatin, with basic amino acids of the histones interacting with negatively charged phosphate groups of the DNA. Approximately 146 base pairs of DNA wrap around a histone core to make up a nucleosome particle, the repeating structural motif of chromatin. Histones are subject to posttranslational acetylation of the α,ε-amino groups of N-terminal lysine residues. The acetylation reaction is catalyzed by enzymes termed histone acetyl transferase (HATs). Acetylation neutralizes the positive charge of the lysine side chain, and is thought to impact chromatin structure in a manner that facilitates transcription (e.g., by allowing transcription factors increased access to DNA). A family of enzymes termed histone deacetylases (HDacs) has been reported to reverse histone acetylation. Eight members of the HDac family, termed HDac1-HDac8, have been reported and proposed as two distinct classes: class I, comprising HDacs 1, 2, 3 and 8, and class II, comprising HDacs 4, 5, 6 and 7. In vivo, the acetylation state of chromatin is thought to be maintained by a dynamic balance between the activities of HATs and HDacs.

Some small molecules have been reported as having HDac inhibitory activity (HDac inhibitors). HDac inhibitors are thought to shift the HDac/HAT balance towards HAT activity, causing an accumulation of acetylated histones. HDac inhibitors have been reported as associated with a diverse range of biological effects, including the induction of cell cycle arrest, terminal differentiation, and apoptosis. HDac inhibitors have also been shown to inhibit tumor formation in animal models, and a number of compounds are currently in Phase I and Phase II clinical trials as potential therapeutics for a variety of cancers. However, to date, the role of HDac inhibitors in the central and peripheral nervous systems has not been fully elucidated.

Citation of the above documents is not intended as an admission that any of the foregoing is pertinent prior art. All statements as to the date or representation as to the contents of these documents is based on the information available to the applicant and does not constitute any admission as to the correctness of the dates or contents of these documents.

### BRIEF SUMMARY OF THE DISCLOSURE

Disclosed herein are compositions and methods for the prophylaxis and treatment of diseases, conditions and injuries of the central and peripheral nervous systems by stimulating or increasing neurogenesis. Aspects of the methods, and activities of the compositions, include increasing or potentiating neurogenesis in cases of a disease, disorder, or condition of the nervous system. Embodiments of the disclosure include methods of treating a neurodegenerative disorder, neurological trauma including brain or central nervous system trauma and/or recovery therefrom, depression, anxiety, psychosis, learning and memory disorders, and ischemia of the central and/or peripheral nervous systems. In other embodiments, the disclosed methods are used to improve cognitive outcomes and treat epilepsy.

In one aspect, methods of modulating, such as by stimulating or increasing, neurogenesis are disclosed. The neurogenesis may be at the level of a cell or tissue. The cell or tissue may be present in an animal subject or a human being, or alternatively be in an in vitro or ex vivo setting. In some embodiments, neurogenesis is stimulated or increased in a neural cell or tissue, such as that of the central or peripheral nervous system of an animal or human being. In other embodiments, neurogenesis may be potentiated in a neural cell or tissue. In cases of an animal or human, the methods may be practiced in connection with one or more disease, disorder, or condition of the nervous system as present in the animal or human subject. Thus, embodiments disclosed herein include methods of treating a disease, disorder, or condition by administering at least one neurogenesis modulating agent having inhibitory activity against histone deacetylase (HDac) activity. The agent is hereinafter referred to as a “neurogenic HDac inhibitor” or a “neuromodulating HDac inhibitor” or an “HDac inhibitory agent.”

While an HDac inhibitory agent may be considered a “direct” agent in that it has direct activity against an HDac by interactions therewith, the disclosure includes an HDac inhibitory agent that may be considered an “indirect” agent in that it does not directly interact with an HDac. Thus, an indirect agent acts on an HDac indirectly, or via production, generation, stability, or retention of an intermediate agent which directly interacts with an HDac.

The HDac inhibitory agent may be used alone or in combination with one or more additional neurogenic agents. The additional neurogenic agent may be another HDac inhibitory agent (direct or indirect) or a neurogenic agent that acts through a mechanism independent from inhibition of HDac activity. An additional neurogenic agent as described herein may be another direct HDac inhibitory agent, another indirect HDac inhibitory agent, or a neurogenic agent that does not act, directly or indirectly, by inhibiting HDac activity. Thus in some embodiments, an additional neurogenic agent is one that acts, directly or indirectly, through a mechanism other than by inhibiting HDac activity.

In a second aspect, the disclosure includes a method of lessening and/or reducing a decline or decrease of cognitive function in a subject or patient treated with a cytotoxic agent and/or condition, such as an anti-proliferative agent and/or condition. In some embodiments, the agent and/or condition is anti-cancer chemotherapy and/or radiation therapy. In some cases, the method may be applied to maintain and/or stabilize cognitive function in the subject or patient. The method may comprise administering an HDac inhibitory agent to a subject or patient in an amount effective to lessen or reduce a decline or decrease of cognitive function due to a cytotoxic agent and/or condition, such as in a subject or patient treated with anti-cancer chemotherapy and/or radiation therapy.

In another aspect, methods of using chemical entities as HDac inhibitory agents to increase neurogenesis, or alleviate a negative effect on cognitive function, are disclosed. In some embodiments, a chemical entity used as an HDac inhibitory agent is a therapeutically or pharmaceutically acceptable reversible HDac inhibitor. Alternatively, an acceptable irreversible HDac inhibitor may also be used in some embodiments of the disclosure. Additional embodiments comprise an inhibitor that crosses the blood brain barrier.

Embodiments of the disclosure include a combination of more than one of the HDac inhibitory agents disclosed herein or known to the skilled person. Of course an HDac inhibitor may be used, either alone or in combination with one or more additional HDac inhibitory agent or other neurogenic agent. Compositions disclosed herein include such combinations of HDac inhibitory agents and one or more other neurogenic agents.

In a further aspect, the disclosed methods include identifying a subject or patient suffering from, or subjected to, one or more diseases, disorders, or conditions, or a symptom thereof, and administering to the patient an HDac inhibitor, alone or in combination with another neurogenic agent, as described herein. In some embodiments, a method includes identification of a subject as in need of an increase in neurogenesis, or the alleviation or moderation in a reduction of cognitive function. The method may then further include administering to the subject or patient, one or more HDac inhibitory agents as disclosed herein. In some cases, the subject is an animal subject, and the patient is a human patient.

Additional embodiments describe a method including administering an HDac inhibitory agent, alone or in combination with another neurogenic agent, to a subject or patient exhibiting the effects of insufficient amounts of, or inadequate levels of, neurogenesis. In some cases, the need for additional neurogenesis is that detectable as a reduction in cognitive function. Embodiments include those where the subject or patient has been subjected to an agent and/or condition that decreases or inhibits neurogenesis. Non-limiting examples of inhibitors of neurogenesis include a cytotoxic agent and/or condition, such as anti-cancer chemotherapy and/or radiation therapy, or opioid receptor agonists, such as a mu receptor subtype agonist like morphine.

In further embodiments, the subject or patient may be demonstrating the effects of insufficient amounts of, or inadequate levels of, neurogenesis, such as through a detectable reduction in cognitive function, due to epilepsy or a condition associated with epilepsy. Thus the disclosure includes a method of lessening or reducing a decline or decrease of cognitive function associated with epilepsy or epileptic seizures by administration of an HDac inhibitory agent as described herein. The method may comprise diagnosing a subject or patient as in need of lessening or reducing a decline or decrease in cognitive function associated with epilepsy or epileptic seizures, and administering an HDac inhibitory agent to the subject or patient to alleviate or moderate the decline or decrease in cognitive function.

In another aspect, a disclosed method provides for administering an HDac inhibitory agent, alone or in combination with another neurogenic agent, to a subject or person that will be subjected to an agent and/or condition that decreases or inhibits neurogenesis. Non-limiting embodiments include those where the subject or person is about to be subject to a decrease or inhibition of neurogenesis because he/she/it i) has been administered anti-cancer chemotherapy and/or radiation therapy; ii) is about to be administered anti-cancer chemotherapy and/or radiation therapy; or iii) is about to be administered morphine or another opioid receptor agonist, like another opiate. Non-limiting examples include administering an HDac inhibitory agent, alone or in combination with another neurogenic agent, to a subject before, simultaneously with, or after the subject is administered anti-cancer chemotherapy and/or radiation therapy in connection with cancer, or administered morphine or other opiate in connection with a surgical procedure.

In some embodiments of the disclosure, the radiation therapy includes radiation applied to the brain of an animal subject or human patient. Radiation of the brain may be in whole (such as by whole brain radiation therapy or WBRT as a non-limiting example) or in part (such as by stereotactic radiosurgery as a non-limiting example).

In other embodiments, the method may be used to moderate or alleviate a mood disorder in the subject or patient described above. Thus the disclosure includes a method of treating a mood disorder in such a subject or patient. Non-limiting examples of the method include those comprising administering an HDac inhibitory agent, optionally in combination with another an HDac inhibitory agent and/or another neurogenic agent, to a subject or patient that is i) under treatment with anti-cancer chemotherapy and/or radiation therapy; or ii) diagnosed as having epilepsy or having seizures associated with epilepsy. The treatment may be with any combination and/or amount that is effective to produce an improvement in said mood disorder.

In yet another aspect, the disclosure includes methods for preparing a population of neural stem cells suitable for transplantation, comprising culturing a population of neural stem cells (NSCs) in vitro, and contacting the cultured neural stem cells with at least one HDac inhibitory agent, optionally in combination with another HDac inhibitory agent and/or another neurogenic agent. In some embodiments, the stem cells are prepared and then transferred to a recipient host animal or human subject. Non-limiting examples of preparation include 1) contact with an HDac inhibitory agent, optionally in combination with another HDac inhibitory agent and/or another neurogenic agent, until the cells have undergone neurogenesis, such as that which is detectable by visual inspection or cell counting, or 2) contact with an HDac inhibitory agent, optionally in combination with another HDac inhibitory agent and/or another neurogenic agent, until the cells have been sufficiently stimulated or induced toward or into neurogenesis. The cells prepared in such a non-limiting manner may be transplanted to a subject, optionally with simultaneous, nearly simultaneous, or subsequent administration of a neurogenic agent, or an HDac inhibitory agent to the subject. While the neural stem cells may be in the form of an in vitro culture or cell line, in other embodiments, the cells may be part of a tissue which is subsequently transplanted into a subject.

In other embodiments, the population of cells may be in vitro or in vivo. The disclosure includes maintaining, stabilizing, stimulating, or increasing neurodifferentiation in such a population of cells. In some embodiments, the population of neural cells is in a tissue in vivo, such as in an animal subject or human patient. In further embodiments, the population of neural cells is in a human patient treated with chemotherapy and/or radiation; a human patient diagnosed as having cancer; or in a human patient diagnosed as having epilepsy, a condition associated with epilepsy, or seizures associated with epilepsy. The method may comprise contacting a cell, a population of cells, or a cell containing tissue with an HDac inhibitory agent to maintain, stabilize stimulate, or increase neurodifferentiation therein. In further embodiments, the method may further comprise contact with an additional neurogenic or neuroproliferative agent to produce both neurodifferentiation and neuroproliferation, and thus neurogenesis, in the cell(s) or tissue or subject/patient. In alternative embodiments, contact with an HDac inhibitory agent is used to treat cell(s) or tissue exhibiting aberrant neuroproliferation, and so possibly neurogenesis.

In a yet further aspect, the disclosure includes methods of stimulating or increasing neurogenesis, or alternatively potentiating neurogenesis, in a subject or patient by administering an HDac inhibitory agent. The administration is optionally in combination with another HDac inhibitory agent and/or another neurogenic agent to produce a neurogenic effect. In some embodiments, the neurogenesis occurs in combination with the stimulation of angiogenesis which provides new cells with access to the circulatory system.

In other embodiments, the method may be used to maintain or reduce the differentiation of neural cells into astrocytes. In some cases, this may be applied as a means to potentiate the differentiation and/or proliferation of neuronal cells. The method may comprise contacting a population of neural cells with an HDac inhibitory agent to maintain or reduce their differentiation into astrocytes. In some embodiments, the cells are in a subject or patient with a nervous system disorder related to disease, cellular degeneration, a psychiatric condition, cellular trauma and/or injury, or another neurologically related condition.

Also within the scope of the disclosure are methods for reducing or preventing neurological damage and/or neurological toxicity, such as upon exposure to a DNA-damaging agent or condition. In some embodiments, the neurological damage and/or toxicity is/are to neural cells that are proliferating, dividing or moving through the mitotic cycle. The methods may comprise administering a neuroprotective amount of an HDac inhibitor as described herein. Additional methods are disclosed for protecting neural cells from the effects of DNA damaging agents or conditions. The methods may comprise administering an HDac inhibitor to a patient or subject who has been, or who will be, exposed to a DNA damaging agent or condition. In some cases, these methods may be used to reduce a negative effect on cognitive function and/or improve a mood disorder as described above and below.

The disclosure further includes a method of protecting neural cells from damage or toxicity. The method may comprise contacting a population of neural cells with an HDac inhibitory agent to protect said cells. In some embodiments, the protection may be in the form of reducing, limiting, or inhibiting the generation of astrocytes or the release of astrocytic factors which negatively affect neuronal differentiation and/or proliferation.

In a related aspect, the disclosure also includes a method to maintain, limit, or reduce the differentiation and/or proliferation of neural cells into astrocytes. The method may comprise contacting a population of neural cells with an HDac inhibitory agent in an effective amount such that the number or type of astrocytes, or cells limited to the astrocytic lineage, are maintained, limited or reduced.

In a further aspect, the disclosure includes a method to reduce or inhibit aberrant differentiation, proliferation and/or migration of neural cells in a tissue. The tissue may be in vitro, such as that for transplantation, or in vivo, such as that in an animal or human being as described herein. The method may comprise administering an HDac inhibitory agent to a subject or patient in an amount effective to decrease or limit aberrant differentiation and/or migration of neural cells in a tissue. In some embodiments, aberrant differentiation, proliferation and/or migration (and combinations thereof) include unwanted astrogenesis; unwanted or undesirable neurogenesis, or neurogenesis of unwanted or undesired cells, in an area or region of the brain or another part of the central nervous system; and formation of unwanted or undesired neural connections between cells.

As described by the foregoing, some methods of the disclosure include treatment to affect or maintain the cognitive function of a subject or patient. These methods optionally include assessing or measuring cognitive function of the subject or patient before, during, and/or after administration of the treatment to detect or determine the effect thereof on cognitive function. In some embodiments, the methods may comprise i) treating a subject or patient that has been previously assessed for cognitive function and ii) reassessing cognitive function in the subject or patient during or after the course of treatment.

The details of additional embodiments are set forth in the accompanying drawings and the description below. Other features, objects, and advantages of the embodiments will be apparent from the drawings and detailed description, and from the claims.

### BRIEF DESCRIPTION OF THE DRAWINGS

FIG. 1 is a dose-response curve showing the effect of trichostatin A on the differentiation of cultured human neural stem cells (hNSCs) along a neuronal lineage in two experiments, Run A (squares) and Run B (circles). Background media values are subtracted and data is normalized with respect to a neuronal positive control (circles). Trichostatin A significantly promoted neuronal differentiation, with a mean EC₅₀ value of approximately 3.45 nM, and/or inhibited astrocyte differentiation (see, FIG. 2).

FIG. 2 is a dose-response curve showing the effect of trichostatin A on the differentiation of cultured human neural stem cells (hNSCs) along an astrocyte lineage in two experiments, Run A (squares) and Run B (circles). Background media values are subtracted and data is normalized with respect to an astrocyte positive control. Trichostatin A did not show a significant effect on astrocyte differentiation within the range of concentrations tested (EC₅₀ value greater than highest concentration tested (approximately 31.6 nM)). In light of the results shown in FIG. 1, trichostatin A preferentially promotes differentiation of hNSCs along a neuronal lineage but does not promote the production of astrocytes.

FIG. 3 is dose-response curve showing the effect of trichostatin A on the cell count of cultured human neural stem cells (hNSCs). Data is shown as a percent of the basal media cell count. Toxic doses typically cause a reduction of the basal cell count below 80%. Trichostatin A had no detectable toxicity at concentrations up to 31.6 nM.

FIG. 4 is dose-response curve showing the effect of various concentrations of the HDac inhibitor MS-275 on neuronal differentiation of cultured rat neural stem cells (rNSC), measured as activation of the Neurofilament high (NFH) promoter. Results are presented as the percent of positive control.

FIG. 5 is dose-response curve showing the effect of various concentrations of the HDac inhibitor MS-275 on neuronal differentiation of cultured rat neural stem cells (rNSC), measured as activation of the GAP43 promoter. Results are presented as the percent of positive control.

FIG. 6 is dose-response curve showing the effect of various concentrations of the HDac inhibitor Valproic acid (VPA) on neuronal differentiation of cultured rat neural stem cells (rNSC), measured as activation of the Neurofilament high (NFH) promoter. Results are presented as the percent of positive control.

FIG. 7 is dose-response curve showing the effect of various concentrations of the HDac inhibitor Valproic acid (VPA) on neuronal differentiation of cultured rat neural stem cells (rNSC), measured as activation of the GAP43 promoter. Results are presented as the percent of positive control.

FIG. 8 is dose-response curve showing the effect of various concentrations of the HDac inhibitor Apicidin on neuronal differentiation of cultured rat neural stem cells (rNSC), measured as activation of the Neurofilament high (NFH) promoter. Results are presented as the percent of positive control.

FIG. 9 is dose-response curve showing the effect of various concentrations of the HDac inhibitor Apicidin on neuronal differentiation of cultured rat neural stem cells (rNSC), measured as activation of the GAP43 promoter. Results are presented as the percent of positive control.

FIG. 10 is a bar graph showing the proportion of BrdU-positive cells in the dentate gyrus of control rats (vehicle) and rats treated with 300 mg/kg of valproic acid for 28 days. Valproic acid significantly decreased proliferation in the dentate gyrus, as indicated by a significant decrease in the proportion of BrdU-positive cells in rats exposed to valproic acid.

FIG. 11 is a dose-response curve showing the effect of valproic acid on the differentiation of cultured human neural stem cells (hNSCs) along an astrocyte lineage. Background media values are subtracted and data is normalized with respect to an astrocyte positive control. Valproic acid showed no promotion of astrocyte differentiation within the range of concentrations tested (the EC₅₀ value is greater than highest tested concentration of approximately 10.0 μM).

FIG. 12 is a dose-response curve showing the effect of valproic acid on the cell count of cultured human neural stem cells (hNSCs). Data is shown as a percent of the basal media cell count. Toxic doses typically cause a reduction of the basal cell count below 80%. Valproic acid had no detectable toxicity at concentrations up to 10 μM.

FIG. 13 is a graph showing growth of cells over time in the presence or absence of valproic acid. After 14 days of growth in basal media, human neural stem cells proliferated and grew to an average of 164% of the area observed at the beginning of the experiment. In the presence of valproic acid, this growth was inhibited such that the cells occupied, on average, 86% of the starting area.

### DETAILED DESCRIPTION OF MODES OF PRACTICE

“Neurogenesis” is defined herein as proliferation, differentiation, migration and/or survival of a neural cell in vivo or in vitro. In various embodiments, the neural cell is an adult, fetal, or embryonic neural stem cell or population of cells. The cells may be located in the central nervous system or elsewhere in an animal or human being. The cells may also be in a tissue, such as neural tissue. In some embodiments, the neural cell is an adult, fetal, or embryonic progenitor cell or population of cells, or a population of cells comprising a mixture of stem cells and progenitor cells. Neural cells include all brain stem cells, all brain progenitor cells, and all brain precursor cells. Neurogenesis includes neurogenesis as it occurs during normal development, as well as neural regeneration that occurs following disease, damage or therapeutic intervention, such as by the treatment described herein. Neurogenesis also includes the integration of newly produced cells into neural networks to produce functional neural cells.

A “neurogenic agent” is defined as a chemical agent or reagent that can promote, stimulate, or otherwise increase the amount or degree or nature of neurogenesis in vivo or ex vivo or in vitro relative to the amount, degree, or nature of neurogenesis in the absence of the agent or reagent. A “neurogenic agent” may increase the degree and/or nature of neurogenesis in a method described in U.S. Provisional Application No. 60/697,905 to Barlow, hereby incorporated by reference in its entirety. Other methods are known in the art, and are described, e.g., in Hao et al., Journal of Neuroscience, 24(29): 6590-6599 (2004); and Shingo et al., Journal of Neuroscience, 21(24): 9733-9743 (2001), each of which is hereby incorporated by reference. In some embodiments, treatment with a neurogenic agent increases neurogenesis if it promotes neurogenesis by at least about 5%, at least about 10%, at least about 25%, at least about 50%, at least about 100%, at least about 500%, or more in comparison to the amount, degree, and/or nature of neurogenesis in the absence of the agent, under the conditions of the method used to detect or determine neurogenesis. As described herein, an HDac inhibitory agent that promotes, stimulates, or otherwise increases the amount or degree or nature of neurogenesis is a neurogenic agent.

The term “astrogenic” is defined in relation to “astrogenesis” which refers to the activation, proliferation, differentiation, migration and/or survival of an astrocytic cell in vivo or in vitro. Non-limiting examples of astrocytic cells include astrocytes, activated microglial cells, astrocyte precursors and potentiated cells, and astrocyte progenitor and derived cells. In some embodiments, the astrocyte is an adult, fetal, or embryonic astrocyte or population of astrocytes. The astrocytes may be located in the central nervous system or elsewhere in an animal or human being. The astrocytes may also be in a tissue, such as neural tissue. In some embodiments, the astrocyte is an adult, fetal, or embryonic progenitor cell or population of cells, or a population of cells comprising a mixture of stem and/or progenitor cells, that is/are capable of developing into astrocytes. Astrogenesis includes the proliferation and/or differentiation of astrocytes as it occurs during normal development, as well as astrogenesis that occurs following disease, damage or therapeutic intervention.

The term “stem cell” (or neural stem cell (NSC)), as used herein, refers to an undifferentiated cell that is capable of self-renewal and differentiation into neurons, astrocytes, and/or oligodendrocytes.

The term “progenitor cell” (e.g., neural progenitor cell), as used herein, refers to a cell derived from a stem cell that is not itself a stem cell. Some progenitor cells can produce progeny that are capable of differentiating into more than one cell type.

The term “cognitive function” refers to mental processes of an animal or human subject relating to information gathering and/or processing; the understanding, reasoning, and/or application of information and/or ideas; the abstraction or specification of ideas and/or information; acts of creativity, problem-solving, and possibly intuition; and mental processes such as learning, perception, and/or awareness of ideas and/or information. The mental processes are distinct from those of beliefs, desires, and the like. In some embodiments, cognitive function may be assessed, and thus optionally defined, via one or more tests or assays for cognitive function. Non-limiting examples of a test or assay for cognitive function include CANTAB (see for example Fray et al. “CANTAB battery: proposed utility in neurotoxicology.” Neurotoxicol Teratol. 1996; 18(4):499-504), Stroop Test, Trail Making, Wechsler Digit Span, or the CogState computerized cognitive test (see also Dehaene et al. “Reward-dependent learning in neuronal networks for planning and decision making.” Prog Brain Res. 2000;126:217-29; Iverson et al. “Interpreting change on the WAIS-III/WMS-III in clinical samples.” Arch Clin Neuropsychol. 2001;16(2):183-91; and Weaver et al. “Mild memory impairment in healthy older adults is distinct from normal aging.” Brain Cogn. 2006;60(2):146-55).

As used herein, the term “HDac” or “HDac” refers to any member of a family of enzymes that remove acetyl groups from the epsilon-amino groups of lysine residues at the N-terminus of a histone. Unless otherwise indicated by context, the term “histone” is meant to refer to any histone protein, including H1, H2A, H2B, H3, H4, and H5, from any species.

The term “HDac inhibitor” or “HDac inhibitory agent” as used herein includes a neurogenic agent, as defined herein, that inhibits, reduces, or otherwise modulates the deacetylation of histones mediated by a histone deacetylase activity. In various embodiments, administering an HDac inhibitor according to methods provided herein reduces histone deacetylase activity by at least about 50%, at least about 75%, or at least about 90% or more in comparison to the absence of the inhibitor. In further embodiments, histone deacetylase activity is reduced by at least about 95% or by at least about 99% or more. Methods for assessing histone deacetylase activity are known in the art, and are described, e.g., in Richon et al., Methods Enzymol., 376:199-205 (2004), Wegener et al., Mol Genet Metab., 80(1-2): 138-47 (2003), U.S. Pat. No. 6,110,697, and U.S. Patent Publication Nos. 20050227300, 20050118596, 20030161830, 20030224473, 20030082668, 20030013176, and 20040091951, all of which are incorporated herein by reference in their entirety. Methods for assessing histone deacetylase activity in human patients are also known in the art, and are described, e.g., in U.S. Patent Publication No. 20050288227, herein incorporated by reference in its entirety.

The terms “neurogenic HDac inhibitor” and “neuromodulating HDac inhibitor” refer to an HDac inhibitor that is a neurogenesis modulating agent. In some embodiments, administering a neurogenic, or neuromodulating, HDac inhibitor according to methods provided herein modulates neurogenesis in a target tissue and/or cell-type by at least about 50%, at least about 75%, or at least about 90% or more in comparison to the absence of the inhibitor. In further embodiments, neurogenesis is modulated by at least about 95% or by at least about 99% or more.

A neuromodulating HDac inhibitor may be used to inhibit a neural cell's proliferation, division, or progress through the cell cycle. Alternatively, a neuromodulating HDac inhibitor may be used to stimulate survival and/or differentiation in a neural cell. As an additional alternative, a neuromodulating HDac inhibitor may be used to inhibit, reduce, or prevent astrocyte activation and/or astrogenesis or astrocyte differentiation.

An “HDac inhibitor” or “HDac inhibitory agent” may be a ligand that binds a molecule with HDac activity and has inhibits or reduces HDac activity. In some embodiments, an HDac inhibitor may act by binding an HDac active site in whole or in part. In some embodiments, an HDac inhibitor or inhibits or reduces HDac activity by at least about 5%, at least about 10%, at least about 15%, at least about 20%, at least about 30%, at least about 50%, at least about 75%, at least about 100%, at least about 200%, at least about 300%, at least about 400%, or at least about 500% or more than the amount of activity in the absence of the HDac inhibitor.

“IC₅₀” and “EC₅₀” values are concentrations of a neuromodulating HDac inhibitor that reduce and promote, respectively, neurogenesis or another physiological activity (e.g., the activity of a receptor) to a half-maximal level. IC₅₀ and EC₅₀ values can be assayed in a variety of environments, including cell-free environments, cellular environments (e.g., cell culture assays), multicellular environments (e.g., in tissues or other multicellular structures), and/or in vivo. In some embodiments, neurogenesis modulating agents used in methods provided herein have IC₅₀ or EC₅₀ values of less than about 10 μM, less than about 1 μM, or less than about 0.1 μM or lower. In other embodiments, a neuromodulating HDac inhibitory agent has an IC₅₀ of less than about 50 nM, less than about 10 nM, or less than about 1 nM or lower.

In some embodiments, selectivity of a neuromodulating HDac inhibitor is measured as the ratio of the IC₅₀ or EC₅₀ value for a desired effect (e.g., modulation of neurogenesis or inhibition of HDac activity) relative to the IC₅₀/EC₅₀ value for an undesired effect. In some embodiments, a “selective” neuromodulating HDac inhibitor has a selectivity of less than about 1:2, less than about 1:10, less than about 1:50, or less than about 1:100. In some embodiments, a neuromodulating HDac inhibitor exhibits selective activity in one or more organs, tissues, and/or cell types relative to another organ, tissue, and/or cell type. For example, in some embodiments, a neuromodulating HDac inhibitor selectively modulates neurogenesis and/or HDac activity in a neurogenic region of the brain, such as the hippocampus (e.g., the dentate gyrus), the subventricular zone, and/or the olfactory bulb.

In other embodiments, modulation by an HDac inhibitor is in a region containing neural cells affected by disease or injury, region containing neural cells associated with disease effects or processes, or region containing neural cells affect other event injurious to neural cells. Non-limiting examples of such events include stroke or radiation therapy of the region. In additional embodiments, a neuromodulating HDac inhibitor substantially modulates two or more physiological activities or target molecules, while being substantially inactive against one or more other molecules and/or activities.

In some embodiments, the neuromodulating HDac inhibitor(s) used in the methods described herein has “selective” activity under certain conditions against one or more HDac family members with respect to the degree and/or nature of activity against one or more other HDac members. In other embodiments, a neuromodulating HDac inhibitor useful in methods provided herein is capable, under certain conditions, of “selectively” modulating one or more physiological processes, biological activities and/or target molecules with respect to other processes, activities, or molecules. In further embodiments, selectivity is achieved by administering a neuromodulating HDac inhibitory agent at a dosage and in a manner that produces a concentration in a target organ or tissue that is therapeutically effective against one or more target molecules, while being sub-therapeutic at non-targeted molecules and/or activities. In some embodiments, the concentration of a neuromodulating HDac inhibitor required for a desired level of neurogenesis modulatory activity is at least about 2-fold lower, at least about 5-fold lower, at least about 10-fold lower, or at least about 20-fold lower than the concentration required to produce an undesired biological effect (e.g., undesirable CNS effects, such as those contributing to extrapyramidal or other side effects). Thus in certain embodiments, selective activity of one or more neuromodulating HDac inhibitors results in enhanced efficacy, fewer side effects, lower effective dosages, less frequent dosing, or other desirable attributes.

In other embodiments, a neuromodulating HDac inhibitor as used herein includes a neurogenesis modulating agent, as defined herein, that elicits an observable neurogenic response by producing, generating, stabilizing, or increasing the retention of an intermediate agent which results in the neurogenic response, optionally when contacted with the HDac inhibitor. As used herein, “increasing the retention of” or variants of that phrase or the term “retention” refer to decreasing the degradation of, or increasing the stability of, an intermediate agent.

Thus, HDac inhibitors useful in methods described herein can modulate histone deacetylation directly (e.g., by inhibiting HDac catalytic activity), indirectly (e.g., by modulating the expression, transport, and/or metabolism of an HDac), and/or by another mode of action (e.g., by interacting with histones, DNA, and/or other molecules associated with HDac activity). In some embodiments, the activity of a neurogenic HDac inhibitor may require one or more additional compounds. HDac inhibitors can comprise any type of agent, including, but not limited to, chemical compounds, proteins, peptidomimetics, and antisense molecules or ribozymes.

In some embodiments, an HDac inhibitor useful in methods disclosed herein are substantially inactive, under certain conditions, against one or more molecular targets, such as (i) CNS receptors, including but not limited to, GABA receptors, opioid receptors (e.g., mu, delta, and kappa opioid receptors), muscarinic receptors (e.g., m1-m5 receptors), histaminergic receptors, phencyclidine receptors, dopamine receptors, alpha and beta-adrenoceptors, sigma receptors (type-1 and type-2), and 5HT-1 and 5-HT-2 receptors; (ii) kinases, including but not limited to, Mitogen-activated protein kinase, PKA, PKB, PKC, CK-2; c-Met, JAK, SYK, KDR, FLT-3, c-Kit, Aurora kinase, CDK kinases (e.g., CDK4/cyclin D, CDK2/cyclin E, CDK2/cyclin A, CDK1/cyclin B), and TAK-1; (iii) other enzymes, including but not limited to, phosphatases, phosphodiesterases, and the like; and/or (iv) receptor-associated ion channels (e.g., calcium, chloride, potassium, and the like).

In some embodiments, an HDac inhibitor disclosed herein exhibit selectivity for the inhibition of one or more classes and/or subtypes of HDacs relative to one or more other classes and/or subtypes of HDacs. For example, in some embodiments, an HDac inhibitor inhibits one or more HDacs, while being substantially inactive with respect to one or more additional HDacs.

In some cases, the selectivity of an HDac inhibitor results in improved efficacy, fewer side effects, lower effective dosages, less frequent dosing, and/or other desirable effects relative to non-selective neurogenesis modulating agents, due, e.g., to the targeting of molecules and/or activities that are differentially expressed in particular tissues and/or cell-types.

The disclosed embodiments include methods of modulating neurogenesis by contacting one or more neural cells with an HDac inhibitory agent optionally in combination with another HDac inhibitory agent and/or another neurogenic agent. The amount of an HDac inhibitory agent, optionally in combination with another HDac inhibitory agent and/or another neurogenic agent, may be selected to be effective to produce an improvement in a treated subject, or detectable neurogenesis in vitro. In some embodiments, the amount is one that also minimizes clinical side effects seen with administration of the inhibitor to a subject. The amount of an HDac inhibitory agent used in vivo may be about 50%, about 45%, about 40%, about 35%, about 30%, about 25%, about 20%, about 18%, about 16%, about 14%, about 12%, about 10%, about 8%, about 6%, about 4%, about 2%, or about 1% or less of the maximum tolerated dose for a subject, such as where another HDac inhibitory agent and/or another neurogenic agent is used in combination. This is readily determined for each HDac inhibitory agent that has been in clinical use or testing, such as in humans.

An HDac inhibitory agent may also be used to lessening or reducing a decline or decrease of cognitive function in a subject or patient treated with anti-cancer chemotherapy and/or radiation therapy. In some embodiments, such a method comprises administering an HDac inhibitory agent to a subject or patient to lessen or reduce a decline or decrease of cognitive function due to anti-cancer chemotherapy and/or radiation therapy. In other embodiments, the method comprises administering an HDac inhibitory agent to a subject or patient that has been assessed for cognitive function. The assessment may be used to determine a background or baseline measurement against which a subsequent reduction in cognitive function may be compared.

In further embodiments, the method comprises i) administering an HDac inhibitory agent to a subject or patient to lessen or reduce a decline or decrease of cognitive function due to anti-cancer chemotherapy and/or radiation therapy and ii) assessing cognitive function in the subject or patient. The assessment may be made at a subsequent time point to measure cognitive function for comparison to a control or standard value (or range) in subjects or patients treated with the same anti-cancer chemotherapy and/or radiation therapy in the absence of an HDac inhibitory agent. This may be used to assess the efficacy of the HDac inhibitory agent in alleviating the reduction in cognitive function caused by the anti-cancer chemotherapy and/or radiation therapy that produces a decline or decrease of cognitive function.

These methods may be applied in cases where anti-cancer chemotherapy and/or radiation therapy in a subject or patient produces a decline or decrease in cognitive function. Without being bound by theory, and offered to improve the understanding of the invention, such a reduction in cognitive function may be due to cytotoxic, neurotoxic, and/or anti-proliferative effects of the anti-cancer chemotherapy and/or radiation therapy. These effects may be moderated or alleviated by the methods comprising administering an HDac inhibitory agent in combination with the anti-cancer chemotherapy and/or radiation therapy. The combination may be used to lessen or reduce the decline or decrease of cognitive function in a treated subject or patient.

Methods to lessen or reduce reductions in cognitive function may also be used to maintain or stabilize cognitive function in a treated subject or patient. In some embodiments, the maintenance or stabilization may be at a level, or thereabouts, present in a subject or patient in the absence of anti-cancer therapy and/or radiation therapy. In alternative embodiments, the maintenance or stabilization may be at a level, or thereabouts, present in a subject or patient as a result of anti-cancer therapy and/or radiation therapy.

In further embodiments, and if compared to a reduced level of cognitive function due to anti-cancer chemotherapy and/or radiation therapy, a method of the invention may be for enhancing or improving the reduced cognitive function in a subject or patient. The method may comprise administering an HDac inhibitory agent to a subject or patient to enhance or improve a decline or decrease of cognitive function due to anti-cancer chemotherapy and/or radiation therapy. The administering may be in combination with the anti-cancer chemotherapy and/or radiation therapy as described herein.

Administration of an HDac inhibitory agent may be before, after, or concurrent with, another agent, condition, or therapy. In some embodiments, the combination may be of an HDac inhibitory agent and a cytotoxic agent and/or condition, such as an anti-proliferative agent and/or condition. In additional embodiments, the agent and/or condition is anti-cancer chemotherapy and/or radiation therapy. Non-limiting examples of such methods include those wherein the chemotherapy comprises administration of a kinase inhibitor or other therapy independent of HDac inhibition. Additional non-limiting examples of such methods include those wherein the subject or patient is a human being diagnosed as having cancer or undergoing treatment for cancer.

Non-limiting examples of cancer include carcinomas and sarcomas as well as those arising from hematological sources, such as lymphomas, leukemias, and myelomas. Non-limiting examples of carcinomas include adenocarcinoma, basal cell carcinoma, squamous cell carcinoma, and transitional cell carcinoma. Non-limiting examples of sarcoma include angiosarcoma, chondrosarcoma, epitheliod sarcoma, Ewings sarcoma, fibrosarcoma, gastrointestinal stromal tumor, Kaposi's Sarcoma, leiomyosarcoma, liposarcoma, malignant schwannoma or neurosarcoma or neurofibrosarcoma, mesenchymoma, osteosarcoma, rhabdomyosarcoma, or synovial cell sarcoma. Other non-limiting examples of cancer include solid tumors and astrocytoma, choroid plexus carcinoma, ependymoma, germ cell cancer, glioblastoma multiforme, glioma, hemangiopericytoma, medulloblastoma, malignant meningioma, mixed oligoastrocytoma, neuroblastoma, neurocytoma, oligodendroglioma, neuroectodermal tumor, melanoma, and mixed adenosquamous carcinoma.

In some embodiments of the invention, the HDac inhibitory agent is trichostatin A, apicidin, MS-275, FK228, SAHA, or valproic acid. In other embodiments, the HDac inhibitory agent is a composition comprising one or more of trichostatin A, apicidin, MS-275, FK228, SAHA, or valproic acid, or a derivative of one of these three agents. Non-limiting examples of valproic acid derivatives include isovalerate, valerate, or valproate. The positive recitation (above and below) of possible HDac inhibitory agents to treat conditions disclosed herein is intended to include, within the disclosure, embodiments with the explicit exclusion of one or more of the agents. As would be recognized by the skilled person, a description of the whole of a plurality of alternative agents necessarily includes and describes subsets of the possible alternatives, or the part remaining with the exclusion of one or more of the alternatives.

In addition to treatment of a subject or patient undergoing anti-cancer chemotherapy and/or radiation therapy, an HDac inhibitory agent may also be used to lessening or reducing a decline or decrease of cognitive function due to epilepsy, a condition associated with epilepsy, or seizures associated with epilepsy. In some embodiments, such a method comprises i) diagnosing a subject or patient as in need of lessening or reducing a decline or decrease in cognitive function due to epilepsy, a condition associated with epilepsy, or seizures associated with epilepsy, and ii) administering an HDac inhibitory agent to the subject or patient. The administration may be with any HDac inhibitory agent in an amount sufficient or effective to reduce a decline or decrease of cognitive function in the subject or patient. In some embodiments, the subject or patient is a human being diagnosed as having epilepsy, a condition associated with epilepsy, or seizures associated with epilepsy.

In other embodiments, the method comprises administering an HDac inhibitory agent, other than valproic acid, to the subject or patient. Again, the HDac inhibitory agent and amount thereof may be any that is sufficient or effective to reduce a decline or decrease of cognitive function in the subject or patient.

In a method relating to epilepsy, a condition associated with epilepsy, or seizures associated with epilepsy, the method may comprise administering an HDac inhibitory agent to a subject or patient that has been assessed for cognitive function. Like in the case of a subject or patient treated with anti-cancer chemotherapy and/or radiation therapy the assessment may be used to determine a background or baseline measurement against which a subsequent reduction in cognitive function may be compared. Alternatively, the assessment may be made at a time subsequent to administration of an HDac inhibitory agent to measure cognitive function for comparison to a control or standard value (or range) in subjects or patients not treated with an HDac inhibitory agent. This may be used to assess the efficacy of the HDac inhibitory agent in alleviating the reduction in cognitive function associated with epilepsy, a condition associated with epilepsy, or seizures associated with epilepsy.

Of course, such a method to lessen or reduce a reduction in cognitive function related to epilepsy and epileptic seizures may also be used to maintain or stabilize cognitive function in a treated subject or patient. In some embodiments, the maintenance or stabilization may be at a level, or thereabouts, present in a subject or patient in the absence of epilepsy, a condition associated with epilepsy, or seizures associated with epilepsy. In other embodiments, the maintenance or stabilization may be at a level, or thereabouts, present in a subject or patient as a result of affliction with epilepsy, a condition associated with epilepsy, or seizures associated with epilepsy.

In further embodiments, and if compared to a reduced level of cognitive function due to epilepsy, a condition associated with epilepsy, or seizures associated with epilepsy, a method of the disclosure may be for enhancing or improving the reduced cognitive function in a subject or patient. The method may comprise administering an HDac inhibitory agent to a subject or patient to enhance or improve a decline or decrease of cognitive function due to epilepsy, a condition associated with epilepsy, or seizures associated with epilepsy.

Methods described herein may also be used to treat a subject or patient of the disclosure for a mood disorder. Various mood disorders are described herein. In some embodiments, a method of treating a mood disorder comprises administering an HDac inhibitory agent, optionally in combination with another an HDac inhibitory agent and/or another neurogenic agent, to a subject or patient that is a) under treatment with a cytotoxic anti-cancer therapy or b) diagnosed as having epilepsy, a condition associated with epilepsy, or seizures associated with epilepsy. The administering is of agent(s) in amounts sufficient or effective to produce an improvement in the disorder. Non-limiting examples of mood disorders include depression, anxiety, hypomania, panic attacks, excessive elation, seasonal mood (or affective) disorder, schizophrenia and other psychoses, lissencephaly syndrome, anxiety syndromes, anxiety disorders, phobias, stress and related syndromes, aggression, non-senile dementia, post-pain depression, and combinations thereof.

Where a neural cell is contacted with an HDac inhibitory agent, the method may be to increase neurodifferentiation. This may be considered a method to potentiate a neural cell for proliferation and thus neurogenesis. Thus the disclosure includes a method of maintaining, stabilizing, stimulating, or increasing neurodifferentiation in a cell or tissue. The method may comprise contacting a cell or tissue with an HDac inhibitory agent to maintain, stabilize stimulate, or increase neurodifferentiation in the cell or tissue.

In some embodiments, the method may further comprise contacting the cell or tissue with an additional neurogenic agent, such as one that stimulates or increases proliferation or cell division in a neural cell. A method comprising such a combination may be used to produce neurogenesis (in this case both neurodifferentiation and proliferation) in a population of neural cells. In some cases, the cell or tissue is in an animal subject or a human patient. In additional embodiments, the cell or tissue is in a human patient treated with chemotherapy and/or radiation; a human patient diagnosed as having cancer; or in a human patient diagnosed as having epilepsy, a condition associated with epilepsy, or seizures associated with epilepsy. Alternatively, the subject or patient is in need of neurogenesis or has been diagnosed with a disease, condition, or injury of the central or peripheral nervous system as described herein.

In further embodiments, the cell or tissue exhibits decreased neurogenesis or is subjected to an agent or condition which decreases or inhibits neurogenesis as described herein. In yet additional embodiments, the cell or tissue exhibits aberrant neurogenesis or neuroproliferation, and the method optionally inhibits, reduces, or limits neuroproliferation.

In additional alternative embodiments, a method comprising the contacting of a neural cell with an HDac inhibitory agent may be used to inhibit neural cell proliferation or division. In some cases, this method protects neural cells from damage or toxicity that only occurs to proliferating, dividing, or cycling cells. Non-limiting examples include the protection of neural cells in a patient subjected to chemotherapy or radiation treatments, such as in the treatment of cancer.

In additional embodiments, the amount or concentration of an HDac inhibitory agent is that which reduces, decreases, or minimizes astrogenesis in a population of neural cells. The disclosure thus includes a method to maintain or reduce the differentiation of neural cells into astrocytes, the cells of or specific to an astrocytic lineage, or the activation of astrocytes. The method may comprise contacting a population of neural cells with an HDac inhibitory agent to maintain or reduce their differentiation into astrocytes or cells of, or specific to, an astrocytic lineage. Alternatively, the contacting may reduce or decrease or minimize the activation of astrocytes.

In further embodiments, the disclosure includes a method of protecting neural cells from damage or toxicity. The method may comprise contacting a population of neural cells with an HDac inhibitory agent to protect the cells. In alternative embodiments, the method may include limitation or inhibition of the level or amount of differentiation of the protected cells into astrocytes.

The amount or concentration of an HDac inhibitory agent may be any that is effective in lowering the amount of astrocyte differentiation and/or astrocyte activation. In some embodiments, the amount or concentration is the minimum necessary to produce a desired, or minimum, level of suppression or reduction in astrogenesis. In other embodiments, the amount or concentration is that which reduces, decreases, or minimizes astrogenesis in a population of neural cells treated with the HDac inhibitory agent and an additional neurogenic agent. In some cases, this may be applied in embodiments where the additional neurogenic agent, even at a reduced or minimum amount or concentration to produce a neurogenic effect, also produces an astrogenic effect.

Methods to limit astrogenesis may be used on any population of neural cells, including cells in a tissue of an animal subject or human patient. The cells or tissue may be in vitro or in vivo. In some embodiments, the cells are in a subject or patient with a nervous system disorder related to disease, cellular degeneration, a psychiatric condition, cellular trauma and/or injury, or another neurologically related condition as described herein. Optionally, the condition is not epilepsy. In other embodiments, the cells are in in a human patient as described in the foregoing methods.

Therefore, the HDac inhibitory agent may be used in some embodiments to reduce or avoid the inhibition of beneficial neurogenesis by a combination of an HDac inhibitory agent and one or more additional neurogenic agents. In some embodiments, the HDac inhibitory agent is used as a neurogenic sensitizing agent, such as one which has no detectable or measurable astrogenic activity. As a non-limiting example, a subject in need of the combination is administered an HDac inhibitory agent as a neurogenic sensitizing agent and an additional neurogenic agent to produce neurogenesis in said subject.

The amount or concentration of an HDac inhibitory agent is an effective one which does not induce an unacceptable level or degree of astrogenesis. Non-limiting examples of such an amount or concentration include amounts which do not increase, or actually decrease, the level of astrogenesis. The level of astrogenesis may be that relative to the amount in the absence of the HDac inhibitory agent or relative to that amount in combination with an additional neurogenic agent in vitro or in vivo. In other embodiments, the amount of astrogenesis with an HDac inhibitor agent, alone or in combination as described herein, may be no more than about 5%, about 10%, about 15%, about 20%, about 25%, about 30%, about 35%, about 40%, about 45%, about 50%, about 55%, about 60%, about 65%, about 70%, or about 75% or higher than in comparison to the absence of the HDac inhibitory agent (alone or in combination).

In further embodiments, the disclosure includes a method to reduce or inhibit aberrant differentiation and/or migration of neural cells in a tissue. Non-limiting examples of aberrant differentiation, proliferation and/or migration (in all combinations) include unwanted or undesired astrogenesis; unwanted or undesirable neurogenesis, or neurogenesis of unwanted or undesired cells, in an area or region of the brain or another part of the central nervous system; and formation of unwanted or undesired neural connections between cells. The formation or proliferation of dopaminergic as opposed to GABAergic (or gabaergic), or vice versa, neurons is a non-limiting example of neurogenesis of undesired cells. The disclosed method may comprise contacting the cells of a tissue with an HDac inhibitory agent to reduce or inhibit aberrant differentiation, proliferation, and/or migration of neural cells in or into the tissue. In some cases, the contact is with a tissue in vivo, such as by administering an HDac inhibitory agent to a subject or patient to reduce or inhibit aberrant differentiation, proliferation, and/or migration of neural cells in or into the tissue. The subject or patient may be any as described for the foregoing methods.

The amount of an HDac inhibitory agent may be an amount that also potentiates or sensitizes, such as by activating or inducing cells to differentiate, a population of neural cells for neurogenesis. The degree of potentiation or sensitization for neurogenesis may be determined with use of the combination in any appropriate neurogenesis assay, including, but not limited to, the neuronal differentiation assay described herein. In some embodiments, the amount of HDac inhibitory agent is the highest amount which produces no detectable neuroproliferation in vitro but yet produces neurogenesis, or a measurable shift in efficacy in promoting neurogenesis in vitro, when used in combination with a neurogenic agent. In other embodiments, the amount of HDac inhibitory agent used in vivo may be about 50%, about 45%, about 40%, about 35%, about 30%, about 25%, about 20%, about 18%, about 16%, about 14%, about 12%, about 10%, about 8%, about 6%, about 4%, about 2%, or about 1% or less of the maximum tolerated dose for a subject. Non-limiting examples of subjects include both human beings and animals in assays for behavior linked to neurogenesis. Exemplary animal assays include those described herein.

The amount of an HDac inhibitory agent may be an amount selected to be effective to produce an improvement in a treated subject, or detectable neurogenesis in vitro, when used in combination with an additional neurogenic agent. In some embodiments, such as in the case of known neurogenic agents, the amount is one that minimizes clinical side effects seen with administration of the agent to a subject. The amount of neurogenic sensitizing agent used in vivo may be about 50%, about 45%, about 40%, about 35%, about 30%, about 25%, about 20%, about 18%, about 16%, about 14%, about 12%, about 10%, about 8%, about 6%, about 4%, about 2%, or about 1% or less of the maximum tolerated dose for a subject. This is readily determined for each HDac inhibitory agent disclosed herein as well as those that have been in clinical use or testing, such as in humans.

In other embodiments, the amount of HDac inhibitory agent is the highest amount which produces no detectable neuroproliferation in vitro, including in animal (or non-human) models for behavior linked to neurogenesis, but yet produces neurogenesis, or a measurable shift in efficacy in promoting neurogenesis in the in vitro assay, when used in combination with an additional neurogenic agent. Alternative embodiments include amounts of HDac inhibitory agent and additional neurogenic agent which produce about 1%, about 2%, about 4%, about 6%, about 8%, about 10%, about 12%, about 14%, about 16%, about 18%, about 20%, about 25%, about 30%, about 35%, or about 40% or more of the neurogenesis seen with the amount that produces the highest level of neurogenesis in an in vitro assay.

In another aspect, the disclosed embodiments include methods of using an HDac inhibitory agent, optionally in combination with another HDac inhibitory agent and/or another neurogenic agent, at a level at which neurogenesis occur. The amount of an HDac inhibitory agent, optionally in combination with another HDac inhibitory agent and/or another neurogenic agent, may be any that is effective to produce neurogenesis, optionally with reduced or minimized amounts of astrogenesis. In some embodiments, the amount may be the lowest needed to produce a desired, or minimum, level of detectable neurogenesis or beneficial effect.

In methods of increasing neurogenesis by contacting cells with HDac inhibitory agent, optionally in combination with another neurogenic agent, the cells may be in vitro or in vivo. In some embodiments, the cells are present in a tissue or organ of a subject animal or human being. The cells are those capable of neurogenesis, such as to result, whether by direct differentiation or by proliferation and differentiation, in differentiated neuronal or glial cells. Representative, and non-limiting examples of non-HDac inhibitory agents for use in the disclosed embodiments are provided below.

In applications to an animal or human being, the embodiments relate to a method of bringing cells into contact with an HDac inhibitory agent, optionally in combination with another HDac inhibitory agent and/or another neurogenic agent, in effective amounts to result in an increase in neurogenesis in comparison to the absence of the HDac inhibitory agent or combination. A non-limiting example is in the administration of the HDac inhibitory agent to the animal or human being. Such contacting or administration may also be described as exogenously supplying the HDac inhibitory agent to a cell or tissue.

In some embodiments, the term “animal” or “animal subject” refers to a non-human mammal, such as a primate, canine, or feline. In other embodiments, the terms refer to an animal that is domesticated (e.g. livestock) or otherwise subject to human care and/or maintenance (e.g. zoo animals and other animals for exhibition). In other non-limiting examples, the terms refer to ruminants or carnivores, such as dogs, cats, birds, horses, cattle, sheep, goats, marine animals and mammals, penguins, deer, elk, and foxes.

The disclosed embodiments also relate to methods of treating diseases, disorders, and conditions of the central and/or peripheral nervous systems (CNS and PNS, respectively) by administering an HDac inhibitory agent, optionally in combination with another HDac inhibitory agent and/or another neurogenic agent. As used herein, “treating” includes prevention, amelioration, alleviation, and/or elimination of the disease, disorder, or condition being treated or one or more symptoms of the disease, disorder, or condition being treated, as well as improvement in the overall well being of a patient, as measured by objective and/or subjective criteria. In some embodiments, treating is used for reversing, attenuating, minimizing, suppressing, or halting undesirable or deleterious effects of, or effects from the progression of, a disease, disorder, or condition of the central and/or peripheral nervous systems. In other embodiments, the method of treating may be advantageously used in cases where additional neurogenesis would replace, replenish, or increase the numbers of cells lost due to injury or disease as non-limiting examples.

The amount of an HDac inhibitory agent, optionally in combination with another HDac inhibitory agent and/or another neurogenic agent, may be any that results in a measurable relief of a disease condition like those described herein. As a non-limiting example, an improvement in the Hamilton depression scale (HAM-D) score for depression may be used to determine (such as quantitatively) or detect (such as qualitatively) a measurable level of improvement in the depression of a subject.

Non-limiting examples of symptoms that may be treated with the methods described herein include abnormal behavior, abnormal movement, hyperactivity, hallucinations, acute delusions, combativeness, hostility, negativism, withdrawal, seclusion, memory defects, sensory defects, cognitive defects, and tension. Non-limiting examples of abnormal behavior include irritability, poor impulse control, distractibility, and aggressiveness. Outcomes from treatment with the disclosed methods include improvements in cognitive function or capability in comparison to the absence of treatment.

A number of compounds with HDac inhibitory activity are known in the art (see e.g., Marks et al., J. Natl. Cancer Inst. 92; 1210-1216 (2000) and Miller et al., J. Med. Chem., 46(24); 5097-5115 (2003), incorporated herein by reference) and may be used as an HDac inhibitory agent of the disclosure.

In other embodiments, an HDac inhibitor is a short-chain fatty acid, such as butyric acid, phenylbutyrate (PB), 4-phenylbutyrate (4-PBA), pivaloyloxymethyl butyrate (Pivanex, AN-9), isovalerate, valerate, valproate, valproic acid, propionate, butyramide, isobutyramide, phenylacetate, 3-bromopropionate, or tributyrin as non-limiting examples. Short-chain fatty acid compounds having HDac inhibitory activity are described in U.S. Pat. Nos. 4,988,731, 5,212,326, 4,913,906, 6,124,495, 6,110,970 6,419,953, 6,110,955, 6,043,389, 5,939455, 6,511,678, 6,528,090, 6,528,091, 6,713,086, 6,720,004, U.S. Patent Publication No. 20040087652, Intl. Publication No. WO 02/007722, and in Phiel et al., J Biol Chem., 276(39):36734-41 (2001), Rephaeli et al., Int J Cancer., 116(2):226-35 (2005), Reid et al., Lung Cancer., 45(3):381-6 (2004), Gottlicher et al., 2001, EMBO J., 22(13):3411-20 (2003), and Vaisburg et al., Bioorg Med Chem Lett., 14(1):283-7 (2004).

In further embodiments, an HDac inhibitor is a compound bearing a hydroxyamic acid group, such as suberoylanlide hydroxamic acid (SAHA), trichostatin A (TSA), trichostatin C (TSC), salicylhydroxamic acid, oxamflatin, suberic bishydroxamic acid (SBHA), m-carboxy-cinnamic acid bishydroxamic acid (CBHA), pyroxamide (CAS RN 382180-17-8), diethyl bis-(pentamethylene-N,N-dimethylcarboxamide)malonate (EMBA), azelaic bishydroxamic acid (ABHA), azelaic-1-hydroxamate-9-anilide (AAHA), 6-(3-Chlorophenylureido) carpoic hydroxamic acid, or A-161906 as non-limiting examples. Without being bound by a particular theory, and offered to improve the understanding of the invention, it is believed that hydroxyamic acid groups block catalytic activity by chelating a catalytic zinc ion in the active-site of HDacs (see e.g., Furumai et al., Proc Natl Acad Sci U S A., 98(1):87-92 (2001)).

Hydroxyamic acid compounds having HDac inhibitory activity are described in U.S. Pat. Nos. 6,800,638, 6,784,173, 6,531,472, 6,495,719, 6,512,123, and 6,511,990, U.S. Patent Publication Nos. 20060004041, 20050227976, 20050187261, 20050107348, 20050131018, 20050124679, 20050085507, 20040266818, 20040122079, 20040024067, and 20030018062, Intl. Publication Nos. EP1174438, WO/2004092115, WO/2005019174, WO0052033, WO018045, WO018171, WO0138322, WO0170675, WO9735990, WO9911659, WO0226703, WO0230879 and WO0226696, and in Butler et al., Clin Cancer Res., 7: 962-970 (2001), Richon et al., Proc. Natl. Acad. Sci. USA: 95; 3003-3007 (1998), Kim et al., Oncogene: 18(15); 2461-2470 (1999), Klan et al., Biol Chem., 384(5):777-85 (2003), Yoshida et al., J Biol Chem., 265(28):17174-9 (1990), Suzuli et al., Bioorg Med Chem Lett., 15(2):331-5 (2005), Kelly et al., J Clin Oncol., 23(17):3923-31 (2005), Kelly et al., Clin Cancer Res., 9(10 Pt 1):3578-88 (2003), Sonoda et al., Oncogene, 13(1):143-9 (1996), Richon et al., Proc Natl Acad Sci U S A., 93(12):5705-8 (1996), Jung et al., J. Med. Chem., 42; 4669-4679. (1999), Jung et al., Bioorg. Med. Chem. Lett., 7(13); 1655-1658 (1997), Lavoie et al., Bioorg. Med. Chem. Letters 11, 2847-2850 (2001), Remiszewski et al., J. Med. Chem. 45, 4, 753-757 (2002), Sternson et al., Org. Lett. 3, 26, 4239-4242 (2001), Bouchain et al., J Med Chem., 46(5):820-30 (2003), and Woo et al., J Med Chem., 45(13):2877-85 (2002).

In further embodiments, an HDac inhibitor is a cyclic tetrapeptide, such as Depsipeptide (FK228), FR225497, trapoxin A, apicidin, chlamydocin, or HC-toxin as non-limiting examples. Cyclic tetrapeptides having HDac inhibitory activity are described in U.S. Pat. Nos. 5,922,837, 6,403,555, 6,656,905, 6,399,568, 6,825,317, 6,831,061, U.S. Patent Publication Nos. 20050209134, 20040014647, 20030078369, and 20020120099, and in Kijima et al., J Biol Chem., 268(30):22429-35 (1993), Jose et al., Bioorg Med Chem Lett.,14(21):5343-6 (2004), Xiao et al., Rapid Commun Mass Spectrom., 17(8):757-66 (2003), Furumai et al., Cancer Res., 62(17):4916-21 (2002), Nakajima et al., Exp. Cell Res., 241; 126-133 (1998), Sandor et al., Clin Cancer Res., 8(3):718-28 (2002), Jung et al., J. Med. Chem., 42; 4669-4679. (1999), and Jung et al., Bioorg. Med. Chem. Lett., 7(13); 1655-1658 (1997).

In yet additional embodiments, an HDac inhibitor is a benzamide, such as MS-275. Benzamides having HDac inhibitory activity are described in U.S. Pat. Nos. 6,174,905 and 6,638,530, U.S. Patent Publication Nos. 2004005513, 20050171103, 20050131018, and 20040224991, Intl. Publication Nos. WO/2004082638, WO/2005066151, WO/2005065681, EP 0847992 and JP 258863/96, and in Saito et al., Proc. Natl. Acad. Sci. USA, vol. 96, pp. 4592-4597 (1999); Suzuki et al., J. Med. Chem., vol. 42, pp. 3001-3003 (1999), Ryan et al., J Clin Oncol., 23(17):3912-22 (2005), Pauer et al., Cancer Invest. 22(6):886-96 (2004), and Undevia et al., Ann Oncol., 15(11):1705-11 (2004).

In some embodiments, an HDac inhibitor is depudecin, a sulfonamide anilide (e.g., diallyl sulfide), BL1521, curcumin (diferuloylmethane), CI-994 (N-acetyldinaline), spiruchostatin A, Scriptaid, carbamazepine (CBZ), or a related compound. These and related compounds having HDac inhibitory activity are described in U.S. Pat. No. 6,544,957, and in Lea et al., Int. J. Oncol., 15, 347-352 (1999), Ouwehand et al., FEBS Lett., 579(6):1523-8 (2005), Kraker et al., Mol Cancer Ther. 2(4):401-8 (2003), de Ruijter et al., Biochem Pharmacol., 68(7):1279-88 (2004), Liu et al., Acta Pharmacol Sin., 26(5):603-9 (2005), Fournel et al., Cancer Res., 62: 4325-4330 (2002), Yurek-George et al., J Am Chem Soc., 126(4):1030-1 (2004), Su et al., Cancer Res., 60(12):3137-42 (2000), Beutler et al., Life Sci., 76(26):3107-15 (2005), and Kwon et al., Proc. Natl. Acad. Sci. USA 95, 3356-3361 (1998).

In other embodiments, an HDac inhibitor is a compound comprising a cyclic tetrapeptide group and a hydroxamic acid group. Examples of such compounds are described in U.S. Pat. Nos. 6,833,384 and 6,552,065, and in Nishino et al., Bioorg Med Chem., 12(22):5777-84 (2004), Nishino et al., Org Lett., 5(26):5079-82 (2003), Komatsu et al., Cancer Res., 61(11):4459-66 (2001), Furumai et al., Proc Natl Acad Sci U S A., 98(1):87-92 (2001), Yoshida et al., Cancer Chemotherapy and Pharmacology, 48 Suppl. 1; S20-S26 (2001), and Remiszeski et al., J Med Chem., 46(21):4609-24 (2003).

In further embodiments, an HDac inhibitor is a compound comprising a benzamide group and a hydroxamic acid group. Examples of such compounds are described in Ryu et al., Cancer Lett. Jul. 9, 2005 (epub), Plumb et al., Mol Cancer Ther., 2(8):721-8 (2003), Ragno et al., J Med Chem., 47(6):1351-9 (2004), Mai et al., J Med Chem., 47(5):1098-109 (2004), Mai et al., J Med Chem., 46(4):512-24 (2003), Mai et al., J Med Chem., 45(9):1778-84 (2002), Massa et al., J Med Chem., 44(13):2069-72 (2001), Mai et al., J Med Chem., 48(9):3344-53 (2005), and Mai et al., J Med Chem., 46(23):4826-9 (2003).

In additional embodiments, an HDac inhibitor is a compound described in U.S. Pat. Nos. 6,897,220, 6,888,027, 5,369,108, 6,541,661, 6,720,445, 6,562,995, 6,777,217, or 6,387,673, 6,693,132, or U.S. Patent Publication Nos. 20060020131, 20060058553, 20060058298, 20060058282, 20060052599, 2006004712, 20060030554, 20060030543, 20050288282, 20050245518, 20050148613, 20050107348, 20050026907, 20040214880, 20040214862, 20040162317, 20040157924, 20040157841, 20040138270, 20040072849, 20040029922, 20040029903, 20040023944, 20030125306, 20030083521, 20020143052, 20020143037, 20050197336, 20050222414, 20050176686, 20050277583, 20050250784, 20050234033, 20050222410, 20050176764, 20050107290, 20040043470, 20050171347, 20050165016, 20050159470, 20050143385, 20050137234, 20050137232, 20050119250, 20050113373, 20050107445, 20050107384, 20050096468, 20050085515, 20050032831, 20050014839, 20040266769, 20040254220, 20040229889, 20040198830, 20040142953, 20040106599, 20040092598, 20040077726, 20040077698, 20040053960, 20040002506, 20030187027, 20020177594, 20020161045, 20020119996,20020115826,20020103192, or 20020065282.

In further additional embodiments, an HDac inhibitor is selected from the group consisting of FK228, AN-9, MS-275, CI-994, LAQ-824, SAHA, G2M-777, PXD-101, LBH-589, MGCD-0103, MK0683, pyroxamide, sodium phenylbutyrate, CRA-024781, and derivatives, salts, metabolites, prodrugs, and stereoisomers thereof.

Additional non-limiting examples include a reported HDac inhibitor selected from ONO-2506 or arundic acid (CAS RN 185517-21-9); MGCD0103 (see Gelmon et al. “Phase I trials of the oral histone deacetylase (HDac) inhibitor MGCD0103 given either daily or 3× weekly for 14 days every 3 weeks in patients (pts) with advanced solid tumors.” Journal of Clinical Oncology, 2005 ASCO Annual Meeting Proceedings. 23(16S, June 1 Supplement), 2005: 3147 and Kalita et al. “Pharmacodynamic effect of MGCD0103, an oral isotype-selective histone deacetylase (HDac) inhibitor, on HDac enzyme inhibition and histone acetylation induction in Phase I clinical trials in patients (pts) with advanced solid tumors or non-Hodgkin's lymphoma (NHL)” Journal of Clinical Oncology, 2005 ASCO Annual Meeting Proceedings. 23(16S, Part I of II, June 1 Supplement), 2005: 9631), a reported thiophenyl derivative of benzamide HDac inhibitor as presented at the 97th American Association for Cancer Research (AACR) Annual Meeting in Washington, D.C. in a poster titled “Enhanced Isotype-Selectivity and Antiproliferative Activity of Thiophenyl Derivatives of BenzamideHDac Inhibitors In Human Cancer Cells,” (abstract #4725), and a reported HDac inhibitor as described in U.S. Pat. No. 6,541,661; SAHA or Vorinostat (CAS RN 149647-78-9); PXD101 or PXD 101 or PX 105684 (CAS RN 414864-00-9), CI-994 or Tacedinaline (CAS RN 112522-64-2), MS-275 (CAS RN 209783-80-2), or an inhibitor reported in WO2005/108367.

In yet further embodiments, an HDac inhibitor is a novel HDac inhibitor identified using structure-activity relationships and teachings known in the art and described, e.g., in Miller et al., J. Med. Chem., 46(24); 5097-5115 (2003) and Klan et al., Biol Chem., 384(5):777-85 (2003)), all of which are incorporated herein by reference in their entirety. Methods to assess histone deacetylase activity are known in the art, and are described, e.g., in Richon et al., Methods Enzymol., 376:199-205 (2004), Wegener et al., Mol Genet Metab., 80(1-2): 138-47 (2003), U.S. Pat. No. 6,110,697, and U.S. Patent Publication Nos. 20050118596, 20050227300, 20030161830, 20030224473, 20030082668, 20030013176, and 20040091951), all of which are incorporated herein by reference in their entirety.

In yet additional embodiments, the neurogenic HDac inhibitor is a molecule that inhibits the transcription and/or translation of one or more HDacs. Antisense oligonucleotides and ribozymes that inhibit transcription and/or translation of one or more HDacs are described in U.S. Pat. No. 6,953,783, and U.S. Patent Publication Nos. 20050171042, 20040266718, 20040204373, 20040077578, 20040077084, 20040077083, 20040072770, 20030236204,20030216345, 20030152557, 20030148970, 20030078216, 20020137162, 20020164752, 20020115177, and 20020061860. In some embodiments, HDac activity is inhibited by administering a combination of at least one HDac enzyme inhibitor, and at least one HDac transcriptional inhibitor.

Methods for assessing the nature and/or degree of neurogenesis in vivo and in vitro, for detecting changes in the nature and/or degree of neurogenesis, for identifying neurogenesis modulating agents, for isolating and culturing neural stem cells, and for preparing neural stem cells for transplantation or other purposes are disclosed, for example, in U.S. Provisional Application No. 60/697,905, and U.S. Publication Nos. 2005/0009742 and 2005/0009847, 20050032702, 2005/0031538, 2005/0004046, 2004/0254152, 2004/0229291, and 2004/0185429, all of which are herein incorporated by reference in their entirety.

As disclosed herein, neurogenesis includes the differentiation of neural cells along different potential lineages. In some embodiments, the differentiation of neural stem or progenitor cells is along a neuronal and/or glial cell lineage, optionally to the exclusion of differentiation along an astrocyte lineage.

Compounds described herein include pharmaceutically acceptable salts, derivatives, prodrugs, and metabolites of the compound. For example, the HDac inhibitor Depsipeptide (FK228) can be considered a prodrug, since reduction of an intramolecular disulfide bond of FK228 in vivo (e.g., by glutathione) greatly enhances its inhibitory activity (see e.g., Furumai et al., Cancer Res. Sep. 1, 2002;62(17):4916-21 and U.S. Patent Publication No. 20040053820, herein incorporated by reference). In addition, metabolites of FK228 may include glutathione conjugates which have been isolated from the blood after administration of FK228 and been shown to have potentially higher activity than the parent compound (see e.g., Xiao et al., Rapid Commun Mass Spectrom., 17(8):757-66 (2003), incorporated herein by reference). Other prodrug HDac inhibitors include AN-7 and AN-9, which are metabolized in vivo to form butyric acid, but have higher activity than butyric acid due to enhanced permeability across cell membranes and/or other characteristics (see e.g., Reid et al., Lung Cancer., 45(3):381-6 (2004); Rephaeli et al., Int J Cancer., 116(2):226-35 (2005), incorporated herein by reference). In some embodiments, the HDac inhibitor is administered as a pharmaceutical composition described in U.S. Patent Pub. No. 20060009527. In some embodiments, the HDac inhibitor is administered in a manner and/or composition in which the HDac inhibitor assumes a particular form or conformation, such as the polymorphs of suberoylanilide hydroxamic acid (SAHA) described in U.S. Patent Pub. No. 20040122101. Methods for preparing and administering salts, derivatives, prodrugs, and metabolites of various compounds are well known in the art.

Compounds described herein that contain a chiral center include all possible stereoisomers of the compound, including compositions comprising the racemic mixture of the two enantiomers, as well as compositions comprising each enantiomer individually, substantially free of the other enantiomer. Thus, for example, contemplated herein is a composition comprising the S enantiomer of a compound substantially free of the R enantiomer, or the R enantiomer substantially free of the S enantiomer. If the named compound comprises more than one chiral center, the scope of the present disclosure also includes compositions comprising mixtures of varying proportions between the diastereomers, as well as compositions comprising one or more diastereomers substantially free of one or more of the other diastereomers. By “substantially free” it is meant that the composition comprises less than 25%, 15%, 10%, 8%, 5%, 3%, or less than 1% of the minor enantiomer or diastereomer(s). Methods for synthesizing, isolating, preparing, and administering various stereoisomers are known in the art.

Methods described herein can be used to treat any disease or condition for which it is beneficial to promote or otherwise stimulate or increase neurogenesis. One focus of the methods described herein is to achieve a therapeutic result by increasing neurogenesis. Thus, certain methods described herein can be used to treat any disease or condition susceptible to treatment by increasing neurogenesis.

In other embodiments, the disease or condition being treated is associated with pain and/or addiction, but in contrast to known methods, the disclosed treatments are substantially mediated by increasing neurogenesis. For example, in some embodiments, methods described herein involve increasing neurogenesis ex vivo, such that a composition containing neural stem cells, neural progenitor cells, and/or differentiated neural cells can subsequently be administered to an individual to treat a disease or condition. In some embodiments, methods described herein allow treatment of diseases characterized by pain, addiction, and/or depression to be treated by directly replenishing, replacing, and/or supplementing neurons and/or glial cells. In further embodiments, methods described herein enhance the growth and/or survival of existing neural cells, and/or slow or reverse the loss of such cells in a neurodegenerative condition.

Examples of diseases and conditions treatable by the methods described herein include, but are not limited to, neurodegenerative disorders and neural disease, such as dementias (e.g., senile dementia, memory disturbances/memory loss, dementias caused by neurodegenerative disorders (e.g., Alzheimer's, Parkinson's disease, Parkinson's disorders, Huntington's disease (Huntington's Chorea), Lou Gehrig's disease, multiple sclerosis, Pick's disease, Parkinsonism dementia syndrome), progressive subcortical gliosis, progressive supranuclear palsy, thalmic degeneration syndrome, hereditary aphasia, amyotrophic lateral sclerosis, Shy-Drager syndrome, and Lewy body disease; vascular conditions (e.g., infarcts, hemorrhage, cardiac disorders); mixed vascular and Alzheimer's; bacterial meningitis; Creutzfeld-Jacob Disease; and Cushing's disease.

The disclosed embodiments also provide for the treatment of a nervous system disorder related to neural damage, cellular degeneration, a psychiatric condition, cellular (neurological) trauma and/or injury (e.g., subdural hematoma or traumatic brain injury), toxic chemicals (e.g., heavy metals, alcohol, some medications), CNS hypoxia, or other neurologically related conditions. In practice, the disclosed compositions and methods may be applied to a subject or patient afflicted with, or diagnosed with, one or more central or peripheral nervous system disorders in any combination. Diagnosis may be performed by a skilled person in the applicable fields using known and routine methodologies which identify and/or distinguish these nervous system disorders from other conditions.

Non-limiting examples of nervous system disorders related to cellular degeneration include neurodegenerative disorders, neural stem cell disorders, neural progenitor cell disorders, degenerative diseases of the retina, and ischemic disorders. In some embodiments, an ischemic disorder comprises an insufficiency, or lack, of oxygen or angiogenesis, and non-limiting example include spinal ischemia, ischemic stroke, cerebral infarction, multi-infarct dementia. While these conditions may be present individually in a subject or patient, the disclosed methods also provide for the treatment of a subject or patient afflicted with, or diagnosed with, more than one of these conditions in any combination.

Non-limiting embodiments of nervous system disorders related to a psychiatric condition include neuropsychiatric disorders and affective disorders. As used herein, an affective disorder refers to a disorder of mood such as, but not limited to, depression, post-traumatic stress disorder (PTSD), hypomania, panic attacks, excessive elation, bipolar depression, bipolar disorder (manic-depression), and seasonal mood (or affective) disorder. Other non-limiting embodiments include schizophrenia and other psychoses, lissencephaly syndrome, anxiety syndromes, anxiety disorders, phobias, stress and related syndromes (e.g., panic disorder, phobias, adjustment disorders, migraines), cognitive function disorders, aggression, drug and alcohol abuse, drug addiction, and drug-induced neurological damage, obsessive compulsive behavior syndromes, borderline personality disorder, non-senile dementia, post-pain depression, post-partum depression, and cerebral palsy.

Examples of nervous system disorders related to cellular or tissue trauma and/or injury include, but are not limited to, neurological traumas and injuries, surgery related trauma and/or injury, retinal injury and trauma, injury related to epilepsy, cord injury, spinal cord injury, brain injury, brain surgery, trauma related brain injury, trauma related to spinal cord injury, brain injury related to cancer treatment, spinal cord injury related to cancer treatment, brain injury related to infection, brain injury related to inflammation, spinal cord injury related to infection, spinal cord injury related to inflammation, brain injury related to environmental toxin, and spinal cord injury related to environmental toxin.

Non-limiting examples of nervous system disorders related to other neurologically related conditions include learning disorders, memory disorders, age-associated memory impairment (AAMI) or age-related memory loss, autism, learning or attention deficit disorders (ADD or attention deficit hyperactivity disorder, ADHD), narcolepsy, sleep disorders and sleep deprivation (e.g., insomnia, chronic fatigue syndrome), cognitive disorders, epilepsy, injury related to epilepsy, and temporal lobe epilepsy.

Other non-limiting examples of diseases and conditions treatable by the methods described herein include, but are not limited to, hormonal changes (e.g., depression and other mood disorders associated with puberty, pregnancy, or aging (e.g., menopause)); and lack of exercise (e.g., depression or other mental disorders in elderly, paralyzed, or physically handicapped patients); infections (e.g., HIV); genetic abnormalities (down syndrome); metabolic abnormalities (e.g., vitamin B12 or folate deficiency); hydrocephalus; memory loss separate from dementia, including mild cognitive impairment (MCI), age-related cognitive decline, and memory loss resulting from the use of general anesthetics, chemotherapy, radiation treatment, post-surgical trauma, or therapeutic intervention; and diseases of the of the peripheral nervous system (PNS), including but not limited to, PNS neuropathies (e.g., vascular neuropathies, diabetic neuropathies, amyloid neuropathies, and the like), neuralgias, neoplasms, myelin-related diseases, etc.

Additionally, the disclosed methods provide for the application of an HDac inhibitory agent, optionally in combination with another HDac inhibitory agent and/or another neurogenic agent, to treat a subject or patient for a condition due to the anti-neurogenic effects of an opiate or opioid based analgesic. In some embodiments, the administration of an opiate or opioid based analgesic, such as an opiate like morphine or other opioid receptor agonist, to a subject or patient results in a decrease in, or inhibition of, neurogenesis. The administration of an HDac inhibitory agent, optionally in combination with another HDac inhibitory agent and/or another neurogenic agent, with an opiate or opioid based analgesic would reduce the anti-neurogenic effect. One non-limiting example is administration of an HDac inhibitory agent, optionally in combination with another HDac inhibitory agent and/or another neurogenic agent, with an opioid receptor agonist after surgery (such as for the treating post-operative pain).

So the disclosed embodiments include a method of treating post operative pain in a subject or patient by combining administration of an opiate or opioid based analgesic with an HDac inhibitory agent, optionally in combination with another HDac inhibitory agent and/or another neurogenic agent. The analgesic may have been administered before, simultaneously with, or after an HDac inhibitory agent, alone or in combination with another neurogenic agent. In some cases, the analgesic or opioid receptor agonist is morphine or another opiate.

Other disclosed embodiments include a method to treat or prevent decreases in, or inhibition of, neurogenesis in other cases involving use of an opioid receptor agonist. The methods comprise the administration of an HDac inhibitory agent, optionally in combination with another HDac inhibitory agent and/or another neurogenic agent, as described herein. Non-limiting examples include cases involving an opioid receptor agonist, which decreases or inhibits neurogenesis, and drug addiction, drug rehabilitation, and/or prevention of relapse into addiction. In some embodiments, the opioid receptor agonist is morphine, opium or another opiate.

Compounds and compositions disclosed herein can also be used to treat diseases of the peripheral nervous system (PNS), including but not limited to, PNS neuropathies (e.g., vascular neuropathies, diabetic neuropathies, amyloid neuropathies, and the like), neuralgias, neoplasms, myelin-related diseases, etc.

Other conditions that can be beneficially treated by increasing neurogenesis are known in the art (see e.g., U.S. Publication Nos. 20020106731, 2005/0009742 and 2005/0009847, 20050032702, 2005/0031538, 2005/0004046, 2004/0254152, 2004/0229291, and 2004/0185429, herein incorporated by reference in their entirety).

In some embodiments, an HDac inhibitory agent, optionally in combination with another HDac inhibitory agent and/or another neurogenic agent, used in the methods described herein, is in the form of compositions that include at least one pharmaceutically acceptable excipient. As used herein, the term “pharmaceutically acceptable excipient” includes any excipient known in the field as suitable for pharmaceutical application. Suitable pharmaceutical excipients and formulations are known in the art and are described, for example, in Remington's Pharmaceutical Sciences (19th ed.) (Genarro, ed. (1995) Mack Publishing Co., Easton, Pa.). Preferably, pharmaceutical carriers are chosen based upon the intended mode of administration of an HDac inhibitory agent. The pharmaceutically acceptable carrier may include, for example, disintegrants, binders, lubricants, glidants, emollients, humectants, thickeners, silicones, flavoring agents, and water.

An HDac inhibitory agent may be incorporated with excipients and administered in the form of ingestible tablets, buccal tablets, troches, capsules, elixirs, suspensions, syrups, wafers, or any other form known in the pharmaceutical arts. The pharmaceutical compositions may also be formulated in a sustained release form. Sustained release compositions, enteric coatings, and the like are known in the art. Alternatively, the compositions may be a quick release formulation.

In some embodiments, methods of treatment disclosed herein comprise the step of administering to a mammal an HDac inhibitory agent, optionally in combination with another HDac inhibitory agent and/or another neurogenic agent, for a time and at a concentration sufficient to treat the condition targeted for treatment. The disclosed methods can be applied to individuals having, or who are likely to develop, disorders relating to neural degeneration, neural damage and/or neural demyelination. In some embodiments, a method comprises selecting a population or sub-population of patients, or selecting an individual patient, that is more amenable to treatment and/or less susceptible to side effects than other patients having the same disease or condition. For example, in some embodiments, a sub-population of patients is identified as being more amenable to neurogenesis with an HDac inhibitory agent, optionally in combination with another HDac inhibitory agent and/or another neurogenic agent, by taking a cell or tissue sample from prospective patients, isolating and culturing neural cells from the sample, and determining the effect of an HDac inhibitory agent, optionally in combination with another HDac inhibitory agent and/or another neurogenic agent, on the degree or nature of neurogenesis, thereby allowing selection of patients for whom an HDac inhibitory agent, or combination of neurogenic agents comprising it, has a substantial effect on neurogenesis. Advantageously, the selection step(s) results in more effective treatment for the disease or condition that known methods using the same or similar compounds.

In other embodiments, methods described herein involve modulating neurogenesis ex vivo with an HDac inhibitory agent, such that a composition containing neural stem cells, neural progenitor cells, and/or differentiated neural cells can subsequently be administered to an individual to treat a disease or condition. In some embodiments, the method of treatment comprises the steps of contacting a neural stem cell or progenitor cell with one or more neurogenic HDac inhibitors to modulate neurogenesis, and transplanting the cells into a patient in need of treatment. Methods for transplanting stem and progenitor cells are known in the art, and are described, e.g., in U.S. Pat. Nos. 5,928,947; 5,817,773; and 5,800,539, and PCT Publication Nos. WO 01/176507 and WO 01/170243, all of which are incorporated herein by reference in their entirety. In some embodiments, methods described herein allow treatment of diseases or conditions by directly replenishing, replacing, and/or supplementing damaged or dysfunctional neurons. In further embodiments, methods described herein enhance the growth and/or survival of existing neural cells, and/or slow or reverse the loss of such cells in a neurodegenerative or other condition.

In alternative embodiments, the method of treatment comprises identifying, generating, and/or propagating neural cells ex vivo in contact with an HDac inhibitory agent, optionally in combination with another HDac inhibitory agent and/or another neurogenic agent, and transplanting the cells into a subject. In another embodiment, the method of treatment comprises the steps of contacting a neural stem cell of progenitor cell with an HDac inhibitory agent, optionally in combination with another HDac inhibitory agent and/or another neurogenic agent, to stimulate neurogenesis, and transplanting the cells into a patient in need of treatment. Also disclosed are methods for preparing a population of neural stem cells suitable for transplantation, comprising culturing a population of neural stem cells (NSCs) in vitro, and contacting the cultured neural stem cells with an HDac inhibitory agent, optionally in combination with another HDac inhibitory agent and/or another neurogenic agent, described herein. The disclosure further includes methods of treating the diseases, disorders, and conditions described herein by transplanting such cells into a subject or patient.

Methods described herein may comprise administering to the subject an effective amount of an HDac inhibitory agent, optionally in combination with another HDac inhibitory agent and/or another neurogenic agent, or pharmaceutical composition comprising the HDac inhibitory agent.

In general, an effective amount of compound(s) in the disclosed methods is an amount sufficient, when used as described herein, to stimulate or increase neurogenesis in the subject targeted for treatment when compared to the absence of the compound. An effective amount of a composition may vary based on a variety of factors, including but not limited to, the activity of the active compound(s), the physiological characteristics of the subject, the nature of the condition to be treated, and the route and/or method of administration. General dosage ranges of certain compounds are provided herein and in the cited references based on animal models of CNS diseases and conditions. Various conversion factors, formulas, and methods for determining human dose equivalents of animal dosages are known in the art, and are described, e.g., in Freireich et al., Cancer Chemother Repts 50(4): 219 (1966), Monro et al., Toxicology Pathology, 23: 187-98 (1995), Boxenbaum and Dilea, J. Clin. Pharmacol. 35: 957-966 (1995), and Voisin et al., Reg. Toxicol. Pharmacol., 12(2): 107-116 (1990), which are herein incorporated by reference.

The disclosed methods typically involve the administration of an HDac inhibitory agent, alone or in combination with another neurogenic agent, in a dosage range of 0.001 ng/kg/day to 500 ng/kg/day, or in a dosage range of 0.05 to 200 ng/kg/day. However, as understood by those skilled in the art, the exact dosage of an HDac inhibitory agent used to treat a particular condition will vary in practice due to a wide variety of factors. Accordingly, dosage guidelines provided herein are not intended to be inclusive of the range of actual dosages, but rather provide guidance to skilled practitioners in selecting dosages useful in the empirical determination of dosages for individual patients. Advantageously, methods described herein allow treatment of one or more conditions with reductions in side effects, dosage levels, dosage frequency, treatment duration, safety, tolerability, and/or other factors.

In some embodiments, an effective, neurogenesis modulating amount is an amount that achieves a concentration within the target tissue, using the particular mode of administration, at or above the IC₅₀ or EC₅₀ for activity of target molecule or physiological process. In some cases, the HDac inhibitory HDac inhibitory agent is administered in a manner and dosage that gives a peak concentration of about 1, about 1.5, about 2, about 2.5, about 5, about 10, about 20 or more times the IC₅₀ or EC₅₀ concentration. IC₅₀ and EC₅₀ values and bioavailability data for an HDac inhibitory agent described herein are known in the art, and are described, e.g., in the references cited herein or can be readily determined using established methods. In addition, methods for determining the concentration of a free compound in plasma and extracellular fluids in the CNS, as well pharmacokinetic properties, are known in the art, and are described, e.g., in de Lange et al., AAPS Journal, 7(3): 532-543 (2005). In some embodiments, an HDac inhibitory agents described herein are administered at a frequency of at least about once daily, or about twice daily, or about three or more times daily, and for a duration of at least about 3 days, about 5 days, about 7 days, about 10 days, about 14 days, or about 21 days, or for about 4 weeks or more.

In other embodiments, an effective, neurogenesis modulating amount is a dose that produces a concentration of the HDac inhibitory agent in an organ, tissue, cell, and/or other region of interest that includes the ED₅₀ (the pharmacologically effective dose in 50% of subjects) with little or no toxicity. IC₅₀ and EC₅₀ values for the modulation of neurogenesis can be determined using methods described in U.S. Provisional Application No. 60/697,905 to Barlow et al., filed Jul. 8, 2005, incorporated by reference, or by other methods known in the art. In some embodiments, the IC₅₀ or EC₅₀ concentration for the modulation of neurogenesis is substantially lower than the IC₅₀ or EC₅₀ concentration for activity of the HDac inhibitory agent at non-targeted molecules and/or physiological processes.

In some methods described herein, the application of an HDac inhibitory agent may allow effective treatment with substantially fewer and/or less severe side effects compared to existing treatments. In some embodiments, combination therapy with an HDac inhibitory agent and one or more additional neurogenic agents allows the combination to be administered at dosages that would be sub-therapeutic when administered individually or when compared to other treatments. In other embodiments, each agent in a combination of agents may be present in an amount that results in fewer and/or less severe side effects than that which occurs with a larger amount. Thus the combined effect of the neurogenic agents will provide a desired neurogenic activity while exhibiting fewer and/or less severe side effects overall. In further embodiments, methods described herein allow treatment of certain conditions for which treatment with the same or similar compounds is ineffective using known methods due, for example, to dose-limiting side effects, toxicity, and/or other factors.

Depending on the desired clinical result, the disclosed neurogenic agents or pharmaceutical compositions are administered by any means suitable for achieving a desired effect. Various delivery methods are known in the art and can be used to deliver an agent to a subject or to NSCs or progenitor cells within a tissue of interest. The delivery method will depend on factors such as the tissue of interest, the nature of the compound (e.g., its stability and ability to cross the blood-brain barrier), and the duration of the experiment or treatment, among other factors. For example, an osmotic minipump can be implanted into a neurogenic region, such as the lateral ventricle. Alternatively, compounds can be administered by direct injection into the cerebrospinal fluid of the brain or spinal column, or into the eye. Compounds can also be administered into the periphery (such as by intravenous or subcutaneous injection, or oral delivery), and subsequently cross the blood-brain barrier.

In various embodiments, the disclosed agents or pharmaceutical compositions are administered in a manner that allows them to contact the subventricular zone (SVZ) of the lateral ventricles and/or the dentate gyrus of the hippocampus. Examples of routes of administration include parenteral, e.g., intravenous, intradermal, subcutaneous, oral (e.g., inhalation), transdermal (topical), transmucosal, and rectal administration. Intranasal administration generally includes, but is not limited to, inhalation of aerosol suspensions for delivery of compositions to the nasal mucosa, trachea and bronchioli.

In some embodiments, the compound combinations are administered so as to either pass through or by-pass the blood-brain barrier. Methods for allowing factors to pass through the blood-brain barrier are known in the art, and include minimizing the size of the factor, providing hydrophobic factors which facilitate passage, and conjugating an HDac inhibitory agent, to a carrier molecule that has substantial permeability across the blood brain barrier. In some instances, the combination of compounds can be administered by a surgical procedure implanting a catheter coupled to a pump device. The pump device can also be implanted or be extracorporally positioned. Administration of the HDac inhibitory agent can be in intermittent pulses or as a continuous infusion. Devices for injection to discrete areas of the brain are known in the art. In certain embodiments, the HDac inhibitory agent is administered locally to the ventricle of the brain, substantia nigra, striatum, locus ceruleous, nucleus basalis Meynert, pedunculopontine nucleus, cerebral cortex, and/or spinal cord by, e.g., injection. Methods, compositions, and devices for delivering therapeutics, including therapeutics for the treatment of diseases and conditions of the CNS and PNS, are known in the art.

In some embodiments, an HDac inhibitory agent is modified to facilitate crossing of the gut epithelium. For example, in some embodiments, an HDac inhibitory agent is a prodrug that is actively transported across the intestinal epithelium and metabolized into the active agent in systemic circulation and/or in the CNS.

In some embodiments, the delivery or targeting of an HDac inhibitory agent to a neurogenic region, such as the dentate gyrus or the subventricular zone, enhances efficacy and reduces side effects compared to known methods involving administration with the same or similar compounds.

In other embodiments, an HDac inhibitory agent is conjugated to a targeting domain to form a chimeric therapeutic, where the targeting domain facilitates passage of the blood-brain barrier (as described above) and/or binds one or more molecular targets in the CNS. In some embodiments, the targeting domain binds a target that is differentially expressed or displayed on, or in close proximity to, tissues, organs, and/or cells of interest. In some cases, the target is preferentially distributed in a neurogenic region of the brain, such as the dentate gyrus and/or the SVZ. For example, in some embodiments, an HDac inhibitory agent is conjugated or complexed with the fatty acid docosahexaenoic acid (DHA), which is readily transported across the blood brain barrier and imported into cells of the CNS.

In embodiments to treat subjects and patients, the methods include identifying a patient suffering from one or more disease, disorders, or conditions, or a symptom thereof, and administering to the subject or patient at least one HDac inhibitory agent as described herein. The identification of a subject or patient as having one or more disease, disorder or condition, or a symptom thereof, may be made by a skilled practitioner using any appropriate means known in the field.

In some embodiments, identifying a patient in need of neurogenesis modulation comprises identifying a patient who has or will be exposed to a factor or condition known to inhibit neurogenesis, including but not limited to, stress, aging, sleep deprivation, hormonal changes (e.g., those associated with puberty, pregnancy, or aging (e.g., menopause), lack of exercise, lack of environmental stimuli (e.g., social isolation), diabetes and drugs of abuse (e.g., alcohol, especially chronic use; opiates and opioids; psychostimulants). In some embodiments, the patient has been identified as non-responsive to treatment with primary medications for the condition(s) targeted for treatment (e.g., non-responsive to antidepressants for the treatment of depression), and the neurogenesis modulating HDac inhibitory agent is administered in a method for enhancing the responsiveness of the patient to a co-existing or pre-existing treatment regimen.

In other embodiments, the method or treatment comprises administering a combination of a primary medications for the condition(s) targeted for treatment and an HDac inhibitory agent. For example, in the treatment of depression or related neuropsychiatric disorders, the HDac inhibitory agent may be administered in conjunction with, or in addition to, electroconvulsive shock treatment, a monoamine oxidase modulator, and/or a selective reuptake modulators of serotonin and/or norepinephrine. In some cases, the HDac inhibitory agent has a synergistic effect with an additional therapeutic agent in treating the disease targeted for treatment.

In other embodiments, the patient in need of neurogenesis modulation suffers from premenstrual syndrome, post-partum depression, or pregnancy-related fatigue and/or depression, and the treatment comprises administering a therapeutically effective amount of an HDac inhibitory agent, alone or in combination with another therapeutic agent. Without being bound by any particular theory, and offered to improve understanding of the invention, it is believed that levels of steroid hormones, such as estrogen, are increased during the menstrual cycle during and following pregnancy, and that such hormones can exert a modulatory effect on neurogenesis.

In some embodiments, the patient is a user of a recreational drug including but not limited to alcohol, amphetamines, PCP, cocaine, and opiates. Without being bound by any particular theory, and offered to improve understanding of the invention, it is believed that some drugs of abuse have a modulatory effect on neurogenesis, which is associated with depression, anxiety and other mood disorders, as well as deficits in cognition, learning, and memory. Moreover, mood disorders are causative/risk factors for substance abuse, and substance abuse is a common behavioral symptom (e.g., self medicating) of mood disorders. Thus, substance abuse and mood disorders may reinforce each other, rendering patients suffering from both conditions non-responsive to treatment. Thus, in some embodiments, an HDac inhibitory agent is administered in combination with one or more additional therapeutic agents to treat patients suffering from substance abuse and/or mood disorders. In various embodiments, the one or more additional agents can be an antidepressant, an antipsychotic, a mood stabilizer, or any other agent known to treat one or more symptoms exhibited by the patient. In some embodiments, a neurogenesis modulating agent exerts a synergistic effect with one or more additional agents on the treatment of substance abuse and/or mood disorders in patients suffering from both conditions.

In further embodiments, the patient is on a co-existing and/or pre-existing treatment regimen involving administration of one or more prescription medications having a modulatory effect on neurogenesis. For example, in some embodiments, the patient suffers from chronic pain and is prescribed one or more opiate/opioid medications; and/or suffers from ADD, ADHD, or a related disorder, and is prescribed a psychostimulant, such as ritalin, dexedrine, adderall, or a similar medication which inhibits neurogenesis. Without being bound by any particular theory, and offered to improve understanding of the invention, it is believed that such medications can exert a modulatory effect on neurogenesis, leading to depression, anxiety and other mood disorders, as well as deficits in cognition, learning, and memory. Thus, in some preferred embodiments, an HDac inhibitory agent is administered to a patient who is currently or has recently been prescribed a medication that exerts a modulatory effect on neurogenesis, in order to treat depression, anxiety, and/or other mood disorders, and/or to improve cognition.

In additional embodiments, the patient suffers from chronic fatigue syndrome; a sleep disorder; lack of exercise (e.g., elderly, infirm, or physically handicapped patients); and/or lack of environmental stimuli (e.g., social isolation); and the treatment comprises administering a therapeutically effective amount of an HDac inhibitory agent, alone or in combination with another therapeutic agent.

In more embodiments, the patient is an individual having, or who is likely to develop, a disorder relating to neural degeneration, neural damage and/or neural demyelination.

In yet additional embodiments, identifying a patient in need of neurogenesis modulation comprises selecting a population or sub-population of patients, or an individual patient, that is more amenable to treatment and/or less susceptible to side effects than other patients having the same disease or condition. In some embodiments, identifying a patient amenable to treatment with an HDac inhibitory agent comprises identifying a patient who has been exposed to a factor known to enhance neurogenesis, including but not limited to, exercise, hormones or other endogenous factors, and drugs taken as part of a pre-existing treatment regimen. In some embodiments, a sub-population of patients is identified as being more amenable to neurogenesis modulation with an HDac inhibitory agent by taking a cell or tissue sample from prospective patients, isolating and culturing neural cells from the sample, and determining the effect of one or more HDac inhibitory agents on the degree or nature of neurogenesis of the cells, thereby allowing selection of patients for which the therapeutic agent has a substantial effect on neurogenesis. Advantageously, the selection of a patient or population of patients in need of or amenable to treatment with an HDac inhibitory agent allows more effective treatment of the disease or condition targeted for treatment than known methods using the same or similar compounds.

In some embodiments, the patient has suffered a CNS insult, such as a CNS lesion, a seizure (e.g., electroconvulsive seizure treatment; epileptic seizures), radiation, chemotherapy and/or stroke or other ischemic injury. Without being bound by any particular theory, and offered to improve understanding of the invention, it is believed that some CNS insults/injuries leads to increased proliferation of neural stem cells, but that the resulting neural cells form aberrant connections which can lead to impaired CNS function and/or diseases, such as temporal lobe epilepsy. In other embodiments, an HDac inhibitory agent is administered to a patient who has suffered, or is at risk of suffering, a CNS insult or injury to stimulate neurogenesis. Advantageously, stimulation of the differentiation of neural stem cells with an HDac inhibitory agent activates signaling pathways necessary for progenitor cells to effectively migrate and incorporate into existing neural networks or to block inappropriate proliferation.

In further embodiments, the methods may be used to treat a cell, tissue, or subject which is exhibiting decreased neurogenesis or increased neurodegeneration. In some cases, the cell, tissue, or subject is, or has been, subjected to, or contacted with, an agent that decreases or inhibits neurogenesis. One non-limiting example is a human subject that has been administered morphine or other agent which decreases or inhibits neurogenesis. Non-limiting examples of other agents include opiates and opioid receptor agonists, such as mu receptor subtype agonists, that inhibit or decrease neurogenesis.

Thus in additional embodiments, the methods may be used to treat subjects having, or diagnosed with, depression or other withdrawal symptoms from morphine or other agents which decrease or inhibit neurogenesis. This is distinct from the treatment of subjects having, or diagnosed with, depression independent of an opiate, such as that of a psychiatric nature, as disclosed herein. In further embodiments, the methods may be used to treat a subject with one or more chemical addiction or dependency, such as with morphine or other opiates, where the addiction or dependency is ameliorated or alleviated by an increase in neurogenesis.

In some embodiments, such as those for treating depression and other neurological diseases and conditions, the methods may optionally further comprise use of one or more agents reported as anti-depressant agents. Thus a method may comprise treatment with an HDac inhibitory agent and one or more reported anti-depressant agents as known to the skilled person. Non-limiting examples of such agents include an SSRI (selective serotonine reuptake inhibitor), such as fluoxetine (Prozac®; described, e.g., in U.S. Pat. Nos. 4,314,081 and 4,194,009), citalopram (Celexa; described, e.g., in U.S. Pat. No. 4,136,193), escitalopram (Lexapro; described, e.g., in U.S. Pat. No. 4,136,193), fluvoxamine (described, e.g., in U.S. Pat. No. 4,085,225) or fluvoxamine maleate (CAS RN: 61718-82-9) and Luvox®, paroxetine (Paxil®; described, e.g., in U.S. Pat. Nos. 3,912,743 and 4,007,196), or sertraline (Zoloft®; described, e.g., in U.S. Pat. No. 4,536,518), or alaproclate; the compound nefazodone (Serozone®; described, e.g., in U.S. Pat. No. 4,338,317); a selective norepinephrine reuptake inhibitor (SNRI) such as reboxetine (Edronax®), atomoxetine (Strattera®), milnacipran (described, e.g., in U.S. Pat. No. 4,478,836), sibutramine or its primary amine metabolite (BTS 54 505), amoxapine, or maprotiline; a selective serotonin & norepinephrine reuptake inhibitor (SSNRI) such as venlafaxine (Effexor; described, e.g., in U.S. Pat. No. 4,761,501), and its reported metabolite desvenlafaxine, or duloxetine (Cymbalta; described, e.g., in U.S. Pat. No. 4,956,388); a serotonin, noradrenaline, and dopamine “triple uptake inhibitor”, such as

DOV 102,677 (see Popik et al. “Pharmacological Profile of the “Triple” Monoamine Neurotransmitter Uptake Inhibitor, DOV 102,677.” Cell Mol Neurobiol. Apr. 25, 2006; Epub ahead of print),

DOV 216,303 (see Beer et al. “DOV 216,303, a “triple” reuptake inhibitor: safety, tolerability, and pharmacokinetic profile.” J Clin Pharmacol. 2004 44(12):1360-7),

DOV 21,947 ((+)-1-(3,4-dichlorophenyl)-3-azabicyclo-(3.1.0)hexane hydrochloride), see Skolnick et al. “Antidepressant-like actions of DOV 21,947: a “triple” reuptake inhibitor.” Eur J Pharmacol. 2003 461(2-3):99-104),

NS-2330 or tesofensine (CAS RN 402856-42-2), or NS 2359 (CAS RN 843660-54-8);

and agents like dehydroepiandrosterone (DHEA), and DHEA sulfate (DHEAS), CP-122,721 (CAS RN 145742-28-5).

Additional non-limiting examples of such agents include a tricyclic compound such as clomipramine, dosulepin or dothiepin, lofepramine (described, e.g., in U.S. Pat. No. 4,172,074), trimipramine, protriptyline, amitriptyline, desipramine(described, e.g., in U.S. Pat. No. 3,454,554), doxepin, imipramine, or nortriptyline; a psychostimulant such as dextroamphetamine and methylphenidate; an MAO inhibitor such as selegiline (Emsam®); an ampakine such as CX516 (or Ampalex, CAS RN: 154235-83-3), CX546 (or 1-(1,4-benzodioxan-6-ylcarbonyl)piperidine), and CX614 (CAS RN 191744-13-5) from Cortex Pharmaceuticals; a V1b antagonist such as SSR149415 ((2S,4R)-1-[5-Chloro-1-[(2,4-dimethoxyphenyl)sulfonyl]-3-(2-methoxy-phenyl)-2-oxo-2,3-dihydro-1H-indol-3-yl]-4-hydroxy-N,N-dimethyl-2-pyrrolidine carboxamide),

[1-(beta-mercapto-beta,beta-cyclopentamethylenepropionic acid), 2-O-ethyltyrosine, 4-valine]arginine vasopressin (d(CH2)5[Tyr(Et2)]VAVP (WK 1-1),

9-desglycine[1-(beta-mercapto-beta,beta-cyclopentamethylenepropionic acid), 2-O-ethyltyrosine, 4-valine]arginine vasopressin desGly9d(CH2)5 [Tyr(Et2)]-VAVP (WK 3-6), or

9-desglycine [1-(beta-mercapto-beta,beta-cyclopentamethylenepropionic acid),2-D-(O-ethyl)tyrosine, 4-valine]arginine vasopressin des Gly9d(CH2)5[D-Tyr(Et2)]VAVP (AO 3-21); a corticotropin-releasing factor (CRF) R antagonist such as CP-154,526 (structure disclosed in Schulz et al. “CP-154,526: a potent and selective nonpeptide antagonist of corticotropin releasing factor receptors.” Proc Natl Acad Sci USA. 1996 93(19):10477-82), NBI 30775 (also known as R121919 or 2,5-dimethyl-3-(6-dimethyl-4-methylpyridin-3-yl)-7-dipropylaminopyrazolo[1,5-a]pyrimidine), astressin (CAS RN 170809-51-5), or a photoactivatable analog thereof as described in Bonk et al. “Novel high-affinity photoactivatable antagonists of corticotropin-releasing factor (CRF)” Eur. J. Biochem. 267:3017-3024 (2000), or AAG561 (from Novartis); a melanin concentrating hormone (MCH) antagonist such as 3,5-dimethoxy-N-(1-(naphthalen-2-ylmethyl)piperidin-4-yl)benzamide or (R)-3,5-dimethoxy-N-(1-(naphthalen-2-ylmethyl)-pyrrolidin-3-yl)benzamide (see Kim et al. “Identification of substituted 4-aminopiperidines and 3-aminopyrrolidines as potent MCH-R1 antagonists for the treatment of obesity.” Bioorg Med Chem Lett. Jul. 29, 2006; [Epub ahead of print] for both), or any MCH antagonist disclosed in U.S. Pat. No. 7,045,636 or published U.S. Patent Application US2005/0171098.

Further non-limiting examples of such agents include a tetracyclic compound such as mirtazapine (described, e.g., in U.S. Pat. No. 4,062,848; see CAS RN 61337-67-5; also known as Remeron, or CAS RN 85650-52-8), mianserin (described, e.g., in U.S. Pat. No. 3,534,041), or setiptiline.

Further non-limiting examples of such agents include agomelatine (CAS RN 138112-76-2), pindolol (CAS RN 13523-86-9), antalarmin (CAS RN 157284-96-3), mifepristone (CAS RN 84371-65-3), nemifitide (CAS RN 173240-15-8) or nemifitide ditriflutate (CAS RN 204992-09-6), YKP-10A or R228060 (CAS RN 561069-23-6), trazodone (CAS RN 19794-93-5), bupropion (CAS RN 34841-39-9 or 34911-55-2) or bupropion hydrochloride (or Wellbutrin, CAS RN 31677-93-7) and its reported metabolite radafaxine (CAS RN 192374-14-4), NS2359 (CAS RN 843660-54-8), Org 34517 (CAS RN 189035-07-2), Org 34850 (CAS RN 162607-84-3), vilazodone (CAS RN 163521-12-8), CP-122,721 (CAS RN 145742-28-5), gepirone (CAS RN 83928-76-1), SR58611 (see Mizuno et al. “The stimulation of beta(3)-adrenoceptor causes phosphorylation of extracellular signal-regulated kinases 1 and 2 through a G(s)-but not G(i)-dependent pathway in 3T3-L1 adipocytes.” Eur J Pharmacol. 2000 404(1-2):63-8), saredutant or SR 48968 (CAS RN 142001-63-6), PRX-0023 (N-{3-[4-(4-cyclohexylmethanesulfonylaminobutyl)piperazin-1-yl]phenyl} acetamide, see Becker et al. “An integrated in silico 3D model-driven discovery of a novel, potent, and selective amidosulfonamide 5-HT1A agonist (PRX-00023) for the treatment of anxiety and depression.” J Med Chem. 2006 49(11):3116-35), Vestipitant (or GW597599, CAS RN 334476-46-9), OPC-14523 or VPI-013 (see Bermack et al. “Effects of the potential antidepressant OPC-14523 [1-[3-[4-(3-chlorophenyl)-1-piperazinyl]propyl]-5-methoxy-3,4-dihydro-2-quinolinone monomethanesulfonate] a combined sigma and 5-HT1A ligand: modulation of neuronal activity in the dorsal raphe nucleus.” J Pharmacol Exp Ther. 2004 310(2):578-83), Casopitant or GW679769 (CAS RN 852393-14-7), Elzasonan or CP-448,187 (CAS RN 361343-19-3), GW823296 (see published U.S. Patent Application US2005/0119248), Delucemine or NPS 1506 (CAS RN 186495-49-8), or Ocinaplon (CAS RN 96604-21-6).

Yet additional non-limiting examples of such agents include CX717 from Cortex Pharmaceuticals, TGBA01AD (a serotonin reuptake inhibitor, 5-HT2 agonist, 5-HT1A agonist, and 5-HT1D agonist) from Fabre-Kramer Pharmaceuticals, Inc., ORG 4420 (an NaSSA (noradrenergic/specific serotonergic antidepressant) from Organon, CP-316,311 (a CRF1 antagonist) from Pfizer, BMS-562086 (a CRF1 antagonist) from Bristol-Myers Squibb, GW876008 (a CRF1 antagonist) from Neurocrine/GlaxoSmithKline, ONO-2333Ms (a CRF1 antagonist) from Ono Pharmaceutical Co., Ltd., JNJ-19567470 or TS-041 (a CRF1 antagonist) from Janssen (Johnson & Johnson) and Taisho, SSR 125543 or SSR 126374 (a CRF1 antagonist) from Sanofi-Aventis, Lu AA21004 and Lu AA24530 (both from H. Lundbeck A/S), SEP-225289 from Sepracor Inc., ND7001 (a PDE2 inhibitor) from Neuro3d, SSR 411298 or SSR 101010 (a fatty acid amide hydrolase, or FAAH, inhibitor) from Sanofi-Aventis, 163090 (a mixed serotonin receptor inhibitor) from GlaxoSmithKline, SSR 241586 (an NK2 and NK3 receptor antagonist) from Sanofi-Aventis, SAR 102279 (an NK2 receptor antagonist) from Sanofi-Aventis, YKP581 from SK Pharmaceuticals (Johnson & Johnson), R1576 (a GPCR modulator) from Roche, or ND1251 (a PDE4 inhibitor) from Neuro3d.

In other disclosed embodiments, a reported anti-psychotic agent may be used in combination with an HDac inhibitory agent. Non-limiting examples of a reported anti-psychotic agent as a member of a combination include olanzapine, quetiapine (Seroquel), clozapine (CAS RN 5786-21-0) or its metabolite ACP-104 (N-desmethylclozapine or norclozapine, CAS RN 6104-71-8), reserpine, aripiprazole, risperidone, ziprasidone, sertindole, trazodone, paliperidone (CAS RN 144598-75-4), mifepristone (CAS RN 84371-65-3), bifeprunox or DU-127090 (CAS RN 350992-10-8), asenapine or ORG 5222 (CAS RN 65576-45-6), iloperidone (CAS RN 133454-47-4), ocaperidone (CAS RN 129029-23-8), SLV 308 (CAS RN 269718-83-4), licarbazepine or GP 47779 (CAS RN 29331-92-8), Org 34517 (CAS RN 189035-07-2), ORG 34850 (CAS RN 162607-84-3), Org 24448 (CAS RN 211735-76-1), lurasidone (CAS RN 367514-87-2), blonanserin or lonasen (CAS RN 132810-10-7), Talnetant or SB-223412 (CAS RN 174636-32-9), secretin (CAS RN 1393-25-5) or human secretin (CAS RN 108153-74-8) which are endogenous pancreatic hormones, ABT 089 (CAS RN 161417-03-4), SSR 504734 (see compound 13 in Hashimoto “Glycine Transporter Inhibitors as Therapeutic Agents for Schizophrenia.” Recent Patents on CNS Drug Discovery, 2006 1:43-53), MEM 3454 (see Mazurov et al. “Selective alpha7 nicotinic acetylcholine receptor ligands.” Curr Med Chem. 2006 13(13): 1567-84), a phosphodiesterase 10A (PDE10A) inhibitor such as papaverine (CAS RN 58-74-2) or papaverine hydrochloride (CAS RN 61-25-6), paliperidone (CAS RN 144598-75-4), trifluoperazine (CAS RN 117-89-5), or trifluoperazine hydrochloride (CAS RN 440-17-5).

Additional non-limiting examples of such agents include trifluoperazine, fluphenazine, chlorpromazine, perphenazine, thioridazine, haloperidol, loxapine, mesoridazine, molindone, pimoxide, or thiothixene, SSR 146977 (see Emonds-Alt et al. “Biochemical and pharmacological activities of SSR 146977, a new potent nonpeptide tachykinin NK3 receptor antagonist.” Can J Physiol Pharmacol. 2002 80(5):482-8), SSR181507 ((3-exo)-8-benzoyl-N-[[(2 s)7-chloro-2,3-dihydro-1,4-benzodioxin-1-yl]methyl]-8-azabicyclo[3.2.1]octane-3-methanamine monohydrochloride), or SLV313 (1-(2,3-dihydro-benzo[1,4]dioxin-5-yl)-4-[5-(4-fluorophenyl)-pyridin-3-ylmethyl]-piperazine).

Further non-limiting examples of such agents include Lu-35-138 (a D4/5-HT antagonist) from Lundbeck, AVE 1625 (a CB1 antagonist) from Sanofi-Aventis, SLV 310,313 (a 5-HT2A antagonist) from Solvay, SSR 181507 (a D2/5-HT2 antagonist) from Sanofi-Aventis, GW07034 (a 5-HT6 antagonist) or GW773812 (a D2, 5-HT antagonist) from GlaxoSmithKline, YKP 1538 from SK Pharmaceuticals, SSR 125047 (a sigma receptor antagonist) from Sanofi-Aventis, MEM1003 (a L-type calcium channel modulator) from Memory Pharmaceuticals, JNJ-17305600 (a GLYT1 inhibitor) from Johnson & Johnson, XY 2401 (a glycine site specific NMDA modulator) from Xytis, PNU 170413 from Pfizer, RGH-188 (a D2, D3 antagonist) from Forrest, SSR 180711 (an alpha7 nicotinic acetylcholine receptor partial agonist) or SSR 103800 (a GLYT1 (Type 1 glycine transporter) inhibitor) or SSR 241586 (a NK3 antagonist) from Sanofi-Aventis.

In other disclosed embodiments, a reported anti-psychotic agent may be one used in treating schizophrenia. Non-limiting examples of a reported anti-schizophrenia agent as a member of a combination with an HDac inhibitory agent include molindone hydrochloride (MOBAN®) and TC-1827 (see Bohme et al. “In vitro and in vivo characterization of TC-1827, a novel brain α4β2 nicotinic receptor agonist with pro-cognitive activity.” Drug Development Research 2004 62(1):26-40).

In light of the positive recitation (above and below) of combinations with alternative agents to treat conditions disclosed herein, the disclosure includes embodiments with the explicit exclusion of one or more of the alternative agents. As would be recognized by the skilled person, a description of the whole of a plurality of alternative agents necessarily includes and describes subsets of the possible alternatives, or the part remaining with the exclusion of one or more of the alternatives.

The combination therapy may be of one of the above with an HDac inhibitory agent as described herein to improve the condition of the subject or patient. Non-limiting examples of combination therapy include the use of lower dosages of the above which reduce side effects of the anti-depressant agent when used alone. For example, an anti-depressant agent like fluoxetine or paroxetine or sertraline may be administered at a reduced or limited dose, optionally also reduced in frequency of administration, in combination with an HDac inhibitory agent alone or in combination with another HDac inhibitory agent. The reduced dose or frequency mediates a sufficient anti-depressant effect so that the side effects of the anti-depressant agent alone are reduced or eliminated.

In additional embodiments, such as, but not limited to, treating weight gain, metabolic syndrome, or obesity, and/or to induce weight loss, an HDac inhibitory agent, alone or in combination with another HDac inhibitory agent and/or neurogenic agent, may be used in combination. Non-limiting examples of another agent include those reported for treating weight gain or metabolic syndrome and/or inducing weight loss such as various diet pills that are commercially or clinically available. In some embodiments, the reported agent for treating weight gain, metabolic syndrome, obesity, or for inducing weight loss is orlistat (CAS RN 96829-58-2), sibutramine (CAS RN 106650-56-0) or sibutramine hydrochloride (CAS RN 84485-00-7), phetermine (CAS RN 122-09-8) or phetermine hydrochloride (CAS RN 1197-21-3), diethylpropion or amfepramone (CAS RN 90-84-6) or diethylpropion hydrochloride, benzphetamine (CAS RN 156-08-1) or benzphetamine hydrochloride, phendimetrazine (CAS RN 634-03-7 or 21784-30-5) or phendimetrazine hydrochloride (CAS RN 17140-98-6) or phendimetrazine tartrate, rimonabant (CAS RN 168273-06-1), bupropion hydrochloride (CAS RN: 31677-93-7), topiramate (CAS RN 97240-79-4), zonisamide (CAS RN 68291-97-4), or APD-356 (CAS RN 846589-98-8).

In other non-limiting embodiments, the agent may be fenfluramine or Pondimin (CAS RN 458-24-2), dexfenfluramine or Redux (CAS RN 3239-44-9), or levofenfluramine (CAS RN 37577-24-5); or a combination thereof or a combination with phentermine. Non-limiting examples include a combination of fenfluramine and phentermine (or “fen-phen”) and of dexfenfluramine and phentermine (or “dexfen-phen”).

The combination therapy may be of one of the above with an HDac inhibitory agent as described herein to improve the condition of the subject or patient. Non-limiting examples of combination therapy include the use of lower dosages of the above additional agents, or combinations thereof, which reduce side effects of the agent or combination when used alone. For example, a combination of fenfluramine and phentermine, or phentermine and dexfenfluramine, may be administered at a reduced or limited dose, optionally also reduced in frequency of administration, in combination with an HDac inhibitory agent alone or in combination with another agent. The reduced dose or frequency may be that which reduces or eliminates the side effects of the combination.

In an additional aspect, methods are disclosed herein for protecting neural stem cells and other neural cells from the effects of agents and conditions that damage and/or modify DNA, referred to herein as “DNA-damaging agents.” DNA damaging agents can include therapeutic drugs and treatment modalities (e.g., chemotherapeutic compounds, radiation therapy), as well as environmental agents and conditions (e.g., UV radiation, pollutants). In some embodiments, the DNA-damaging agent is administered as an anti-cancer therapy. DNA-damaging agents can cause a host of undesirable CNS side effects, e.g., by targeting healthy neural cells, in addition to cells targeted for treatment. For example, in some embodiments, the DNA-damaging agent is an anti-cancer therapeutic that selectively targets rapidly dividing cells. Methods for detecting proliferating cells are known in the art, and include, e.g., measuring the incorporation of DNA analogues (such as BrdU), as described in Example 5. DNA-damaging therapeutics that target dividing cells have enhanced efficacy against malignant cells, but can also exert harmful effects against proliferating neural stem and/or progenitor cells, as well as tissues having a high proportion of proliferating cells (e.g., tissues with a high “growth fraction”), such as the hippocampus and the lateral ventricles. Moreover, in some embodiments, a DNA-damaging agent can exert deleterious effects (e.g., “bystander effects”) against surrounding cells that are not directly effected by the DNA-damaging agent. Thus, therapeutics and other agents that target dividing cells can cause widespread neurotoxicity and/or neurological damage.

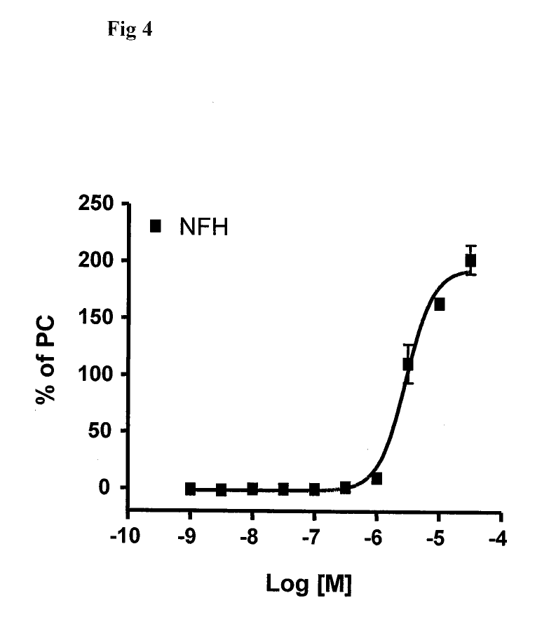

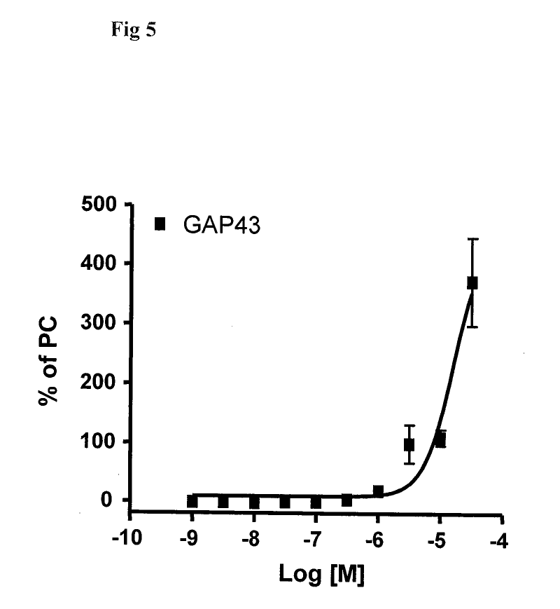

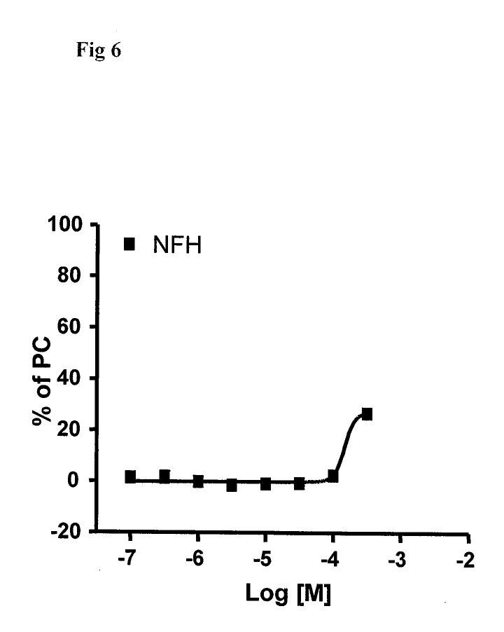

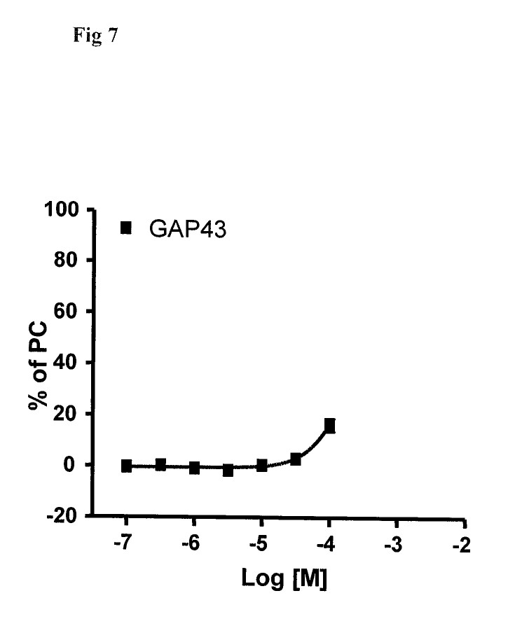

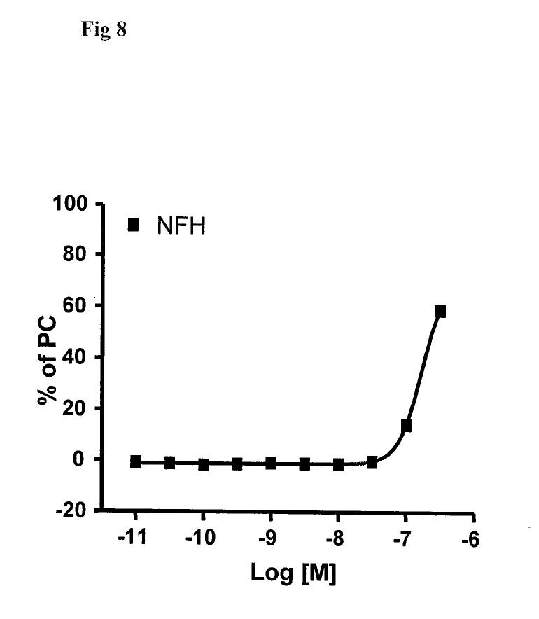

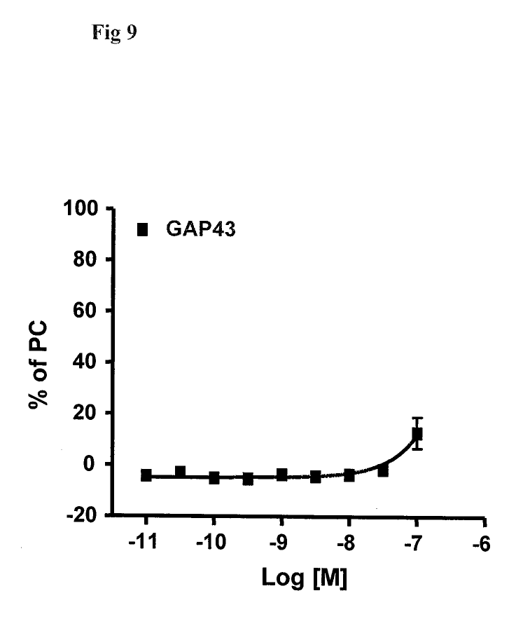

Without being bound by a particular theory, and offered to improve understanding of the invention, it is believed that neuromodulating HDac inhibitors can protect against toxic effects of DNA-damaging agents by inhibiting proliferation and/or promoting differentiation of neural stem and/or progenitor cells, and/or modulating other aspects of neurogenesis. Thus, in various embodiments, methods are disclosed for preventing or ameliorating the neurotoxic effects of a DNA-damaging agent, wherein the methods comprise administering, to a patient that has been and/or will be exposed to a DNA-damaging agent, an effective amount of one or more neuromodulating HDac inhibitors. In some embodiments, the neuromodulating HDac inhibitor stimulates differentiation along a neuronal lineage, for example as shown for MS-275, apicidin, and valproic acid in FIGS. 4-9. In further embodiments, the neuromodulating HDac inhibitor stimulates neuronal differentiation, and also inhibits proliferation of NSCs, for example as shown for valproic acid in FIGS. 6-7, 10 and 13.

Neuromodulating HDac inhibitors can be administered prior to, concurrently, and/or after exposure to a DNA-damaging agent, e.g., as an adjunctive therapy to a primary treatment, as a combination therapy comprising a DNA-damaging agent and a neuromodulating HDac inhibitor, and/or as a stand-alone therapy to treat patients otherwise exposed to a DNA-damaging agent. Advantageously, administration of one or more neuromodulating HDac inhibitors according to methods provided herein can reduce or prevent neurological damage mediated by DNA-damaging agents, and/or treat one or more symptoms of neurotoxicity, including but not limited to, dementia, hallucinations, delusions, depression, anxiety, speech impairments, short-term and/or long-term memory impairments (such as amnesia), learning disabilities, insomnia and other sleep disorders, malaise, confusion, agitation, unresponsiveness, seizures, vertigo, headaches, aphasia, ataxia, tremors, and paraesthesia.

In some embodiments, methods are disclosed for enhancing the therapeutic efficacy of a DNA-damaging agent, wherein the method comprises administering a neuromodulating HDac inhibitor to a patient who has received or will receive a DNA-damaging agent. In various embodiments, administering a neuromodulating HDac inhibitor reduces undesirable side effects, improves the therapeutic index, enhances patient compliance, and/or otherwise improves the effectiveness of a DNA-damaging agent in treating a tumor or other condition. In other embodiments, methods disclosed herein are used to prevent neurotoxic effects of a DNA-damaging agent used to treat a brain tumor, such as a malignant glioma. The treatment of brain tumors with DNA-damaging agents can lead to toxic effects on neural stem cells and/or other neural cells surrounding targeted tumor cells. Moreover, DNA-damaging agents used to treat brain tumors can have particularly widespread CNS side effects, e.g., because malignant cells often disseminate throughout the brain producing numerous neoplastic foci, and neural stem cells have a strong tendency to migrate to the site of tumors. Advantageously, methods provided herein reduce neurological damage and/or neurotoxic side effects associated with the treatment of brain tumors with DNA-damaging therapies, leading to increased well-being of patients as well as enhancements in the overall effectiveness of the therapies.

Neuromodulating HDac inhibitors described herein can be used to treat or prevent the neurotoxic effects of any DNA-damaging agent having activity against neural cells. Non-limiting examples of DNA-damaging agents include topoisomerase inhibitors, such as epipodophyllotoxins (e.g., etoposide (VP16) and teniposide (VM-26)), irinotecan (CPT-11), SN-38, topotecan, and camptothecan; alkylating agents, such as alkyl sulfonates (e.g., busulfan), ethyleneimines and methylmelamines (e.g., hexamethylmelamine, altretamine, thiotepa), nitrogen mustards (e.g., cyclophosphamide, mechlorethamine, uramustine, melphalan, chlorambucil), nitrosoureas (e.g., carmustine, streptozocin), and triazenes (e.g., dacarbazine, temozolomide); antimetabolites, such as 5-fluorouracil (5-FU), S-1 (Tegafur), 5-fluoro-deoxyuridine (5-FudR), 5-ethynyluracil, 5-iododeoxyuridine (5-ludR), 5-bromodeoxyuridine (5-BudR), fluorouridine triphosphate (5-FUTP), fluorodeoxyuridine monophosphate (5-dFUMP), arabinosyl cytosine (ara-C), 5-azacytidine (5-AC), 2′,2′-difluoro-2′-deoxycytidine (dFdC), gemcitabine hydrochlorine (Gemzar), armofur, doxifluridine, emitefur, floxuridine, pentostatin, capecitabine, mercaptopurine, azathiopurine, and thioguanine; anthracyclines, such as doxorubicin, mitoxantrone, daunosamine, daunorubicin, idarubicin, epirubicin, pirarubicin, zorubicin, mitoxantrone, actinimycin D, and carubicin; platinum derivatives, such as cisplatin (CDDP), trans analogue of cisplatin, carboplatin, iproplatin, tetraplatin, and oxaliplatin; radioisotopes, such as ²¹²Bi, ¹³I, ⁹⁰Y, and ¹⁸⁶Re; as well as ifosfamide, rebeccamycin, adriamycin, and bleomycin.

Other non-limiting examples of nucleic acid damaging treatments and conditions include radiation e.g., ultraviolet (UV), infrared (IR), or α-, β-, or γ-radiation, environmental or pathological shock, e.g., hyperthermia, hypoxia, seizure (e.g., epileptic seizure), and the like. Additional nucleic acid-damaging agents and conditions are known in the art, and are within the scope of the instant methods.

At high concentrations (e.g., concentrations greater than about 50 μM or about 100 μM, or greater than about 250 μM, about 500 μM, or more), some HDac inhibitors exert cytotoxic effects against tumor cells and/or other cell types, and can therefore also be used as cancer therapeutics. Without being bound by a particular theory, and offered to improve understanding of the invention, it is believed that at high concentrations, some HDac inhibitors can cause the modification of cellular DNA, e.g., by rendering DNA more accessible to endogenous DNA-damaging agents, such as reactive-oxygen species (ROS), whereas at low concentrations, their effects are mediated by non-toxic mechanisms, such as regulating gene expression and/or other cellular responses. Thus, in some embodiments, a neuromodulating HDac inhibitor is administered in manner such that the compound is present in the CNS and/or a tissue or other region of interest at substantially lower concentrations than those that produce cytotoxic effects against neural cells and/or other cell types.

Non-limiting examples of concentrations of HDac inhibitory agents used in a method disclosed herein include those below about 50 μM, below about 40 μM, below about 30 μM, below about 25 μM, below about 20 μM, below about 15 μM, below about 10 μM, below about 5 μM, below about 1 μM, below about 0.5 μM, below about 0.25 μM, below about 0.1 μM, below about 0.05 μM, below about 0.04 μM, below about 0.03 μM, below about 0.02 μM, below about 0.01 μM, below about 0.005 μM, below about 0.0025 μM, below about 0.001 μM, or a concentration below which an HDac inhibitory agent does not produce detectable (or unwanted or undesirable) cytotoxicity. A skilled person may of course select and use the corresponding amounts of an HDac inhibitory agent to administer and produce the above concentrations in vivo.

The disclosure includes combination therapy, where one or more HDac inhibitory agents and one or more other compounds are used together to produce neurogenesis. When administered as a combination, the therapeutic compounds can be formulated as separate compositions that are administered at the same time or sequentially at different times, or the therapeutic compounds can be given as a single composition. The methods of the disclosure are not limited in the sequence of administration.

Instead, the disclosure includes methods wherein treatment with an HDac inhibitory agent, and another HDac inhibitory agent and/or neurogenic agent occurs over a period of more than about 48 hours, more than about 72 hours, more than about 96 hours, more than about 120 hours, more than about 144 hours, more than about 7 days, more than about 9 days, more than about 11 days, more than about 14 days, more than about 21 days, more than about 28 days, more than about 35 days, more than about 42 days, more than about 49 days, more than about 56 days, more than about 63 days, more than about 70 days, more than about 77 days, more than about 12 weeks, more than about 16 weeks, more than about 20 weeks, or more than about 24 weeks or more. In some embodiments, treatment by administering an HDac inhibitory agent, occurs at least about 12 hours, such as at least about 24, or at least about 36 hours, before administration of another neurogenic agent. Following administration of an HDac inhibitory agent, further administrations may be of only the other neurogenic agent in some embodiments of the disclosure. In other embodiments, further administrations may be of only the HDac inhibitory agent.

In some embodiments, an HDac inhibitory agent has a synergistic effect with the one or more additional active agents. In some embodiments, one or more additional agents potentiate the effect of an HDac inhibitory agent and/or an HDac inhibitory agent potentiates the effect of the additional agent(s). Methods for assessing synergism, potentiation, and other combined pharmacological effects are known in the art, and described, e.g., in Chou and Talalay, Adv Enzyme Regul., 22:27-55 (1984).

In some non-limiting embodiments, combination therapy with a neurogenesis modulating HDac inhibitor and one or more additional agents, or with two or more neurogenesis modulating HDac inhibitors results in a enhanced efficacy, safety, therapeutic index, and/or tolerability, and/or reduced side effects (frequency, severity, or other aspects), dosage levels, dosage frequency, and/or treatment duration. Examples of compounds useful in combinations provided herein are provided below, for which structures, synthetic processes, safety profiles, biological activity data, methods for determining biological activity, pharmaceutical preparations, and methods of administration are known in the art and/or provided in the cited references, all of which are herein incorporated by reference in their entirety. Dosages of compounds administered in combination with a neurogenesis modulating HDac inhibitor can be, e.g., a dosage within the range of pharmacological dosages established in humans, or a dosage that is a fraction of the established human dosage, e.g., 70%, 50%, 30%, 10%, or less than the establishes human dosage.

In some embodiments, the neurogenic agent combined with an HDac inhibitory agent may be a reported opioid or non-opioid (acts independently of an opioid receptor) agent. In some embodiments, the neurogenic agent is one reported as antagonizing one or more opioid receptors or as an inverse agonist of at least one opioid receptor. A opioid receptor antagonist or inverse agonist may be specific or selective (or alternatively non-specific or non-selective) for opioid receptor subtypes. So an antagonist may be non-specific or non-selective such that it antagonizes more than one of the three known opioid receptor subtypes, identified as OP₁, OP₂, and OP₃ (also know as delta, or δ, kappa, or κ, and mu, or μ, respectively). Thus an opioid that antagonizes any two, or all three, of these subtypes, or an inverse agonist that is specific or selective for any two or all three of these subtypes, may be used as the neurogenic agent in the practice. Alternatively, an antagonist or inverse agonist may be specific or selective for one of the three subtypes, such as the kappa subtype as a non-limiting example.

Non-limiting examples of reported opioid antagonists include naltrindol, naloxone, naloxene, naltrexone, JDTic (Registry Number 785835-79-2; also known as 3-isoquinolinecarboxamide, 1,2,3,4-tetrahydro-7-hydroxy-N-[(S1)-1-[[(3R,4R)-4-(3-hydroxyphenyl)-3,4-dimethyl-1-piperidinyl]methyl]-2-methylpropyl]-dihydrochloride, (3R)-(9CI)), nor-binaltorphimine, or buprenorphine. In some embodiments, a reported selective kappa opioid receptor antagonist compound, as described in US 20020132828, U.S. Pat. No. 6,559,159, and/or WO 2002/053533, may be used. All three of these documents are herein incorporated by reference in their entireties as if fully set forth. Further non-limiting examples of such reported antagonists is a compound disclosed in U.S. Pat. No. 6,900,228 (herein incorporated by reference in its entirety); arodyn (Ac[Phe(1,2,3),Arg(4),d-Ala(8)]Dyn A-(1-11)NH(2)) as described in Bennett, et al. (2002) J. Med. Chem. 45:5617-5619); an active analog of arodyn as described in Bennett e al. (2005) J Pept Res. 65(3):322-32; alvimopan; cyprodime (described, e.g., in WO 93/02707); nalmefene (described, e.g., in U.S. Pat. Nos. 3,814,768 and 3,896,226); naltrindole (NTI) (described, e.g., in U.S. Pat. No. 4,816,586) or naltrindole isothiocyanate; nalorphine (described, e.g., in U.S. Pat. Nos. 2,364,833 and 2,891,954) or nalorphine dinicotinate; naltriben (NTB) (described, e.g., in U.S. Pat. No. 4,816,586); DPI-2505 (described, e.g., in U.S. Pat. No. 5,658,908); methiodide; naloxonazine; nalide; nalmexone; b-funaltrexamine (b-FNA); cyclazocine; BNTX; ICI-174,864; LY117413; MR2266; or a compound disclosed in U.S. Pat. Nos. 4,816,586, 4,891,379, 4,191,771, 6,313,312, 6,503,905, or 6,444,679.

In some embodiments, the neurogenic agent used in the methods described herein has “selective” activity (such as in the case of an antagonist or inverse agonist) under certain conditions against one or more opioid receptor subtypes with respect to the degree and/or nature of activity against one or more other opioid receptor subtypes. For example, in some embodiments, the neurogenic agent has an antagonist effect against one or more subtypes, and a much weaker effect or substantially no effect against other subtypes. As another example, an additional neurogenic agent used in the methods described herein may act as an agonist at one or more opioid receptor subtypes and as antagonist at one or more other opioid receptor subtypes. In some embodiments, a neurogenic agent has activity against kappa opioid receptors, while having substantially lesser activity against one or both of the delta and mu receptor subtypes. In other embodiments, a neurogenic agent has activity against two opioid receptor subtypes, such as the kappa and delta subtypes. As non-limiting examples, the agents naloxone and naltrexone have nonselective antagonist activities against more than one opioid receptor subtypes. In certain embodiments, selective activity of one or more opioid antagonists results in enhanced efficacy, fewer side effects, lower effective dosages, less frequent dosing, or other desirable attributes.

An opioid receptor antagonist is an agent able to inhibit one or more characteristic responses of an opioid receptor or receptor subtype. As a non-limiting example, an antagonist may competitively or non-competitively bind to an opioid receptor, an agonist or partial agonist (or other ligand) of a receptor, and/or a downstream signaling molecule to inhibit a receptor's function.

An inverse agonist able to block or inhibit a constitutive activity of an opioid receptor may also be used. An inverse agonist may competitively or non-competitively bind to an opioid receptor and/or a downstream signaling molecule to inhibit a receptor's function. Non-limiting examples of inverse agonists for use in the disclosed methods include ICI-174864 (N,N-diallyl-Tyr-Aib-Aib-Phe-Leu), RTI-5989-1, RTI-5989-23, and RTI-5989-25 (see Zaki et al. J. Pharmacol. Exp. Therap. 298(3): 1015-1020, 2001).

Thus embodiments of the disclosure include a combination of an HDac inhibitory agent with an additional agent such as acetylcholine or a reported modulator of an androgen receptor. Non-limiting examples include the androgen receptor agonists ehydroepiandrosterone (DHEA) and DHEA sulfate (DHEAS).

Alternatively, the neurogenic agent in combination with an HDac inhibitory agent may be an enzymatic inhibitor, such as a reported inhibitor of HMG CoA reductase. Non-limiting examples of such inhibitors include atorvastatin (CAS RN 134523-00-5), cerivastatin (CAS RN 145599-86-6), crilvastatin (CAS RN 120551-59-9), fluvastatin (CAS RN 93957-54-1) and fluvastatin sodium (CAS RN 93957-55-2), simvastatin (CAS RN 79902-63-9), lovastatin (CAS RN 75330-75-5), pravastatin (CAS RN 81093-37-0) or pravastatin sodium, rosuvastatin (CAS RN 287714-41-4), and simvastatin (CAS RN 79902-63-9). Formulations containing one or more of such inhibitors may also be used in a combination. Non-limiting examples include formulations comprising lovastatin such as Advicor (an extended-release, niacin containing formulation) or Altocor (an extended release formulation); and formulations comprising simvastatin such as Vytorin (combination of simvastatin and ezetimibe).

In other non-limiting embodiments, the neurogenic agent in combination with an HDac inhibitory agent may be a reported Rho kinase inhibitor. Non-limiting examples of such an inhibitor include fasudil (CAS RN 103745-39-7); fasudil hydrochloride (CAS RN 105628-07-7); the metabolite of fasudil, which is hydroxyfasudil (see Shimokawa et al. “Rho-kinase-mediated pathway induces enhanced myosin light chain phosphorylations in a swine model of coronary artery spasm.” Cardiovasc Res. 1999 43:1029-1039), Y 27632 (CAS RN 138381-45-0); a fasudil analog thereof such as (S)-Hexahydro-1-(4-ethenylisoquinoline-5-sulfonyl)-2-methyl-1H-1,4-diazepine, (S)-hexahydro-4-glycyl-2-methyl-1-(4-methylisoquinoline-5-sulfonyl)-1H-1,4-diazepine, or (S)-(+)-2-methyl-1-[(4-methyl-5-isoquinoline)sulfonyl]-homopiperazine (also known as H-1152P; see Sasaki et al. “The novel and specific Rho-kinase inhibitor (S)-(+)-2-methyl-1-[(4-methyl-5-isoquinoline)sulfonyl]-homopiperazine as a probing molecule for Rho-kinase-involved pathway.” Pharmacol Ther. 2002 93(2-3):225-32); or a substituted isoquinolinesulfonamide compound as disclosed in U.S. Pat. No. 6,906,061.

Furthermore, the neurogenic agent in combination with an HDac inhibitory agent may be a reported GSK-3 inhibitor or modulator. In some non-limiting embodiments, the reported GSK3-beta modulator is a paullone, such as alsterpaullone, kenpaullone (9-bromo-7,12-dihydroindolo[3,2-d][1]benzazepin-6(5H)-one), gwennpaullone (see Knockaert et al. “Intracellular Targets of Paullones. Identification following affinity purification on immobilized inhibitor.” J Biol Chem. 2002 277(28):25493-501), azakenpaullone (see Kunick et al. “1-Azakenpaullone is a selective inhibitor of glycogen synthase kinase-3 beta.” Bioorg Med Chem Lett. 2004 14(2):413-6), or the compounds described in U.S. Publication No. 20030181439; International Publication No. WO 01/60374; Leost et al., Eur. J. Biochem. 267:5983-5994 (2000); Kunick et al., J Med Chem.; 47(1): 22-36 (2004); or Shultz et al., J. Med. Chem. 42:2909-2919 (1999); an anticonvulsant, such as lithium or a derivative thereof (e.g., a compound described in U.S. Pat. Nos. 1,873,732; 3,814,812; and 4,301,176); valproic acid or a derivative thereof (e.g., valproate, or a compound described in Werstuck et al., Bioorg Med Chem Lett., 14(22): 5465-7 (2004)); lamotrigine; SL 76002 (Progabide), Gabapentin; tiagabine; or vigabatrin; a maleimide or a related compound, such as Ro 31-8220, SB-216763, SB-410111, SB-495052, or SB-415286, or a compound described, e.g., in U.S. Pat. No. 6,719,520; U.S. Publication No. 20040010031; International Publication Nos. WO-2004072062; WO-03082859; WO-03104222; WO-03103663, WO-03095452, WO-2005000836; WO 0021927; WO-03076398; WO-00021927; WO-00038675; or WO-03076442; or Coghlan et al., Chemistry & Biology 7: 793 (2000); a pyridine or pyrimidine derivative, or a related compound (such as 5-iodotubercidin, GI 179186X, GW 784752X and GW 784775X, and compounds described, e.g., in U.S. Pat. Nos. 6489344; 6417185; and 6153618; U.S. Publication Nos. 20050171094; and 20030130289; European Patent Nos. EP-01454908, EP-01454910, EP-01295884, EP-01295885; and EP -01460076; EP-01454900; International Publication Nos. WO 01/70683; WO 01/70729; WO 01/70728; WO 01/70727; WO 01/70726; WO 01/70725; WO-00218385; WO-00218386; WO-03072579; WO-03072580; WO-03027115; WO-03027116; WO-2004078760; WO-2005037800, WO-2004026881, WO-03076437, WO-03029223; WO-2004098607; WO-2005026155; WO-2005026159; WO-2005025567; WO-03070730; WO-03070729; WO-2005019218; WO-2005019219; WO-2004013140; WO-2004080977; WO-2004026229, WO-2004022561; WO-03080616; WO-03080609; WO-03051847; WO-2004009602; WO-2004009596; WO-2004009597; WO-03045949; WO-03068773; WO-03080617; WO 99/65897; WO 00/18758; WO0307073; WO-00220495; WO-2004043953, WO-2004056368, WO-2005012298, WO-2005012262, WO-2005042525, WO-2005005438, WO-2004009562, WO-03037877; WO-03037869; WO-03037891; WO-05012307; WO-05012304 and WO 98/16528; and in Massillon et al., Biochem J 299:123-8 (1994)); a pyrazine derivative, such as Aloisine A (7-n-Butyl-6-(4-hydroxyphenyl)[5H]pyrrolo[2,3-b]pyrazine) or a compound described in International Publication Nos. WO-00144206; WOO 144246; or WO-2005035532; a thiadiazole or thiazole, such as TDZD-8 (Benzyl-2-methyl-1,2,4-thiadiazolidine-3,5-dione); OTDZT (4-Dibenzyl-5-oxothiadiazolidine-3-thione); or a related compound described, e.g., in U.S. Pat. Nos. 6,645,990 or 6,762,179; U.S. Publication No. 20010039275; International Publication Nos. WO 01/56567, WO-03011843, WO-03004478, or WO-03089419; or Mettey, Y., et al., J. Med. Chem. 46, 222 (2003); TWS119 or a related compound, such as a compound described in Ding et al., Proc Natl Acad Sci U S A., 100(13): 7632-7 (2003); an indole derivative, such as a compound described in International Publication Nos. WO-03053330, WO-03053444, WO-03055877, WO-03055492, WO-03082853, or WO-2005027823; a pyrazine or pyrazole derivative, such as a compound described in U.S. Pat. Nos. 6727251, 6696452, 6664247, 666073, 6656939, 6653301, 6653300, 6638926, 6613776, or 6610677; or International Publication Nos. WO-2005002552, WO-2005002576, or WO-2005012256; a compound described in U.S. Pat. Nos. 6719520; 6,498,176; 6,800,632; or 6,872,737; U.S. Publication Nos. 20050137201; 20050176713; 20050004125; 20040010031; 20030105075; 20030008866; 20010044436; 20040138273; or 20040214928; International Publication Nos. WO 99/21859; WO-00210158; WO-05051919; WO-00232896; WO-2004046117; WO-2004106343; WO-00210141; WO-00218346; WO 00/21927; WO 01/81345; WO 01/74771; WO 05/028475; WO 01/09106; WO 00/21927; WO01/41768; WO 00/17184; WO 04/037791; WO-04065370; WO 01/37819; WO 01/42224; WO 01/85685; WO 04/072063; WO-2004085439; WO-2005000303; WO-2005000304; or WO 99/47522; or Naerum, L., et al., Bioorg. Med. Chem. Lett. 12, 1525 (2002); CP-79049, GI 179186X, GW 784752X, GW 784775X, AZD-1080, AR-014418, SN-8914, SN-3728, OTDZT, Aloisine A, TWS119, CHIR98023, CHIR99021, CHIR98014, CHIR98023, 5-iodotubercidin, Ro 31-8220, SB-216763, SB-410111, SB-495052, SB-415286, alsterpaullone, kenpaullone, gwennpaullone, LY294002, wortmannin, sildenafil, CT98014, CT-99025, flavoperidol, or L803-mts.

In yet further embodiments, the neurogenic agent used in combination with an HDac inhibitory agent may be a reported glutamate modulator or metabotropic glutamate (mGlu) receptor modulator. In some embodiments, the reported mGlu receptor modulator is a Group II modulator, having activity against one or more Group II receptors (mGlu₂ and/or mGlu₃). Embodiments include those where the Group II modulator is a Group II agonist. Non-limiting xamples of Group II agonists include: (i) (1S,3R)-1-aminocyclopentane-1,3-dicarboxylic acid (ACPD), a broad spectrum mGlu agonist having substantial activity at Group I and II receptors; (ii) (−)-2-thia-4-aminobicyclo-hexane-4,6-dicarboxylate (LY389795), which is described in Monn et al., J. Med. Chem., 42(6): 1027-40 (1999); (iii) compounds described in US App. No. 20040102521 and Pellicciari et al., J. Med. Chem., 39, 2259-2269 (1996); and (iv) the Group II-specific modulators described below.

Non-limiting examples of reported Group II antagonists include: (i) phenylglycine analogues, such as (RS)-alpha-methyl-4-sulphonophenylglycine (MSPG), (RS)-alpha-methyl-4-phosphonophenylglycine (MPPG), and (RS)-alpha-methyl-4-tetrazolylphenylglycine (MTPG), described in Jane et al., Neuropharmacology 34: 851-856 (1995); (ii) LY366457, which is described in O'Neill et al., Neuropharmacol., 45(5): 565-74 (2003); (iii) compounds described in US App Nos. 20050049243, 20050119345 and 20030157647; and (iv) the Group II-specific modulators described below.

In some non-limiting embodiments, the reported Group II modulator is a Group II-selective modulator, capable of modulating mGlu₂ and/or mGlu₃ under conditions where it is substantially inactive at other mGlu subtypes (of Groups I and III). Examples of Group II-selective modulators include compounds described in Monn, et al., J. Med. Chem., 40, 528-537 (1997); Schoepp, et al., Neuropharmacol., 36, 1-11 (1997) (e.g., 1S,2S,5R,6S-2-aminobicyclohexane-2,6-dicarboxylate); and Schoepp, Neurochem. Int., 24, 439 (1994).

Non-limiting examples of reported Group II-selective agonists include (i) (+)-2-aminobicyclohexane-2,6-dicarboxylic acid (LY354740), which is described in Johnson et al., Drug Metab. Disposition, 30(1): 27-33 (2002) and Bond et al., NeuroReport 8: 1463-1466 (1997), and is systemically active after oral administration (e.g., Grillon et al., Psychopharmacol. (Berl), 168: 446-454 (2003)); (ii) (−)-2-Oxa-4-aminobicyclohexane-4,6-dicarboxylic acid (LY379268), which is described in Monn et al., J. Med. Chem. 42: 1027-1040 (1999) and U.S. Pat. No. 5,688,826. LY379268 is readily permeable across the blood-brain barrier, and has EC₅₀ values in the low nanomolar range (e.g., below about 10 nM, or below about 5 nM) against human mGlu₂ and mGlu₃ receptors in vitro; (iii) (2R,4R)-4-aminopyrrolidine-2,4-dicarboxylate ((2R,4R)-APDC), which is described in Monn et al., J. Med. Chem. 39: 2990 (1996) and Schoepp et al., Neuropharmacolog, 38: 1431 (1999); (iv) (1 S,3S)-1-aminocyclopentane-1,3-dicarboxylic acid ((1S,3S)-ACPD), described in Schoepp, Neurochem. Int., 24: 439 (1994); (v) (2R,4R)-4-aminopyrrolidine-2,4-dicarboxylic acid ((2R,4R)-APDC), described in Howson and Jane, British Journal of Pharmacolog, 139, 147-155 (2003); (vi) (2S,1′S,2′S)-2-(carboxycyclopropyl)-glycine (L-CCG-I), described in Brabet et al., Neuropharmacology 37: 1043-1051 (1998); (vii) (2S,2′R,3′R)-2-(2′,3′-dicarboxycyclopropyl)glycine (DCG-IV), described in Hayashi et al., Nature, 366, 687-690 (1993); (viii) 1S,2S,5R,6S-2-aminobicyclohexane-2,6-dicarboxylate, described in Monn, et al., J. Med. Chem., 40, 528 (1997) and Schoepp, et al., Neuropharmacol., 36, 1 (1997); and (vii) compounds described in US App. No. 20040002478; U.S. Pat. Nos. 6,204,292, 6,333,428, 5,750,566 and 6,498,180; and Bond et al., Neuroreport 8: 1463-1466 (1997).

Non-limiting examples of reported Group II-selective antagonists useful in methods provided herein include the competitive antagonist (2S)-2-amino-2-(1S,2S-2-carboxycycloprop-1-yl)-3-(xanth-9-yl) propanoic acid (LY341495), which is described, e.g., in Kingston et al., Neuropharmacology 37: 1-12 (1998) and Monn et al., J Med Chem 42: 1027-1040 (1999). LY341495 is readily permeably across the blood-brain barrier, and has IC₅₀ values in the low nanomolar range (e.g., below about 10 nM, or below about 5 nM) against cloned human mGlu₂ and mGlu₃ receptors. LY341495 has a high degree of selectivity for Group II receptors relative to Group I and Group III receptors at low concentrations (e.g., nanomolar range), whereas at higher concentrations (e.g., above 1 μM), LY341495 also has antagonist activity against mGlu₇ and mGlu₈, in addition to mGlu_{2/3}. LY341495 is substantially inactive against KA, AMPA, and NMDA iGlu receptors.

Additional non-limiting examples of reported Group II-selective antagonists include the following compounds, indicated by chemical name and/or described in the cited references: (i) α-methyl-L-(carboxycyclopropyl)glycine (CCG); (ii) (2S,3S,4S)-2-methyl-2-(carboxycyclopropyl)glycine (MCCG); (iii) (1R,2R,3R,5R,6R)-2-amino-3-(3,4-dichlorobenzyloxy)-6 fluorobicyclohexane-2,6-dicarboxylic acid (MGS0039), which is described in Nakazato et al., J. Med. Chem., 47(18):4570-87 (2004); (iv) an n-hexyl, n-heptyl, n-octyl, 5-methylbutyl, or 6-methylpentyl ester prodrug of MGS0039; (v) MGS0210 (3-(3,4-dichlorobenzyloxy)-2-amino-6-fluorobicyclohexane-2,6-dicarboxylic acid n-heptyl ester); (vi) (RS)-1-amino-5-phosphonoindan-1-carboxylic acid (APICA), which is described in Ma et al., Bioorg. Med. Chem. Lett., 7: 1195 (1997); (vii) (2S)-ethylglutamic acid (EGLU), which is described in Thomas et al., Br. J. Pharmacol. 117: 70P (1996); (viii) (2S,1′S,2′S,3′R)-2-(2′-carboxy-3′-phenylcyclopropyl)glycine (PCCG-IV); and (ix) compounds described in U.S. Pat. No. 6,107,342 and US App No. 20040006114. APICA has an IC₅₀ value of approximately 30 μM against mGluR₂ and mGluR₃, with no appreciable activity against Group I or Group III receptors at sub-mM concentrations.

In some non-limiting embodiments, a reported Group II-selective modulator is a subtype-selective modulator, capable of modulating the activity of mGlu₂ under conditions in which it is substantially inactive at mGlu₃ (mGlu₂-selective), or vice versa (mGlu₃-selective). Non-limiting examples of subtype-selective modulators include compounds described in U.S. Pat. No. 6,376,532 (mGlu₂-selective agonists) and US App No. 20040002478 (mGlu₃-selective agonists). Additional non-limiting examples of subtype-selective modulators include allosteric mGlu receptor modulators (mGlu₂ and mGlu₃) and NAAG-related compounds (mGlu₃), such as those described below.

In other non-limiting embodiments, a reported Group II modulator is a compound with activity at Group I and/or Group III receptors, in addition to Group II receptors, while having selectivity with respect to one or more mGlu receptor subtypes. Non-limiting examples of such compounds include: (i) (2S,3S,4S)-2-(carboxycyclopropyl)glycine (L-CCG-1) (Group I/Group II agonist), which is described in Nicoletti et al., Trends Neurosci. 19: 267-271 (1996), Nakagawa, et al., Eur. J. Pharmacol., 184, 205 (1990), Hayashi, et al., Br. J. Pharmacol., 107, 539 (1992), and Schoepp et al., J. Neurochem., 63., page 769-772 (1994); (ii) (S)-4-carboxy-3-hydroxyphenylglycine (4C₃HPG) (Group II agonist/Group I competitive antagonist); (iii) gamma-carboxy-L-glutamic acid (GLA) (Group II antagonist/Group III partial agonist/antagonist); (iv) (2S,2′R,3′R)-2-(2,3-dicarboxycyclopropyl)glycine (DCG-IV) (Group II agonist/Group III antagonist), which is described in Ohfune et al, Bioorg. Med. Chem. Lett., 3: 15 (1993); (v) (RS)-a-methyl-4-carboxyphenylglycine (MCPG) (Group I/Group II competitive antagonist), which is described in Eaton et al., Eur. J. Pharmacol., 244: 195 (1993), Collingridge and Watkins, TiPS, 15: 333 (1994), and Joly et al., J. Neurosci., 15: 3970 (1995); and (vi) the Group II/III modulators described in U.S. Pat. Nos. 5,916,920, 5,688,826, 5,945,417, 5,958,960, 6,143,783, 6,268,507, 6,284,785.

In some non-limiting embodiments, the reported mGlu receptor modulator comprises (S)-MCPG (the active isomer of the Group I/Group II competitive antagonist (RS)-MCPG) substantially free from (R)-MCPG. (S)-MCPG is described, e.g., in Sekiyama et al., Br. J. Pharmacol., 117: 1493(1996) and Collingridge and Watkins, TiPS, 15:333(1994).

Additional non-limiting examples of reported mGlu modulators useful in methods disclosed herein include compounds described in U.S. Pat. Nos. 6,956,049, 6,825,211, 5,473,077, 5,912,248, 6,054,448, and 5,500,420; US App Nos. 20040077599, 20040147482, 20040102521, 20030199533 and 20050234048; and Intl Pub/App Nos. WO 97/19049, WO 98/00391, and EP0870760.

In some non-limiting embodiments, the reported mGlu receptor modulator is a prodrug, metabolite, or other derivative of N-Acetylaspartylglutamate (NAAG), a peptide neurotransmitter in the mammalian CNS that is a highly selective agonist for mGluR₃ receptors, as described in Wroblewska et al., J. Neurochem., 69(1): 174-181 (1997). In other embodiments, the mGlu modulator is a compound that modulates the levels of endogenous NAAG, such as an inhibitor of the enzyme N-acetylated-alpha-linked-acidic dipeptidase (NAALADase), which catalyzes the hydrolysis of NAAG to N-acetyl-aspartate and glutamate. Examples of NAALADase inhibitors include 2-PMPA (2-(phosphonomethyl)pentanedioic acid), which is described in Slusher et al., Nat. Med., 5(12): 1396-402 (1999); and compounds described in J. Med. Chem. 39: 619 (1996), US Pub. No. 20040002478, and U.S. Pat. Nos. 6,313,159, 6,479,470, and 6,528,499. In some embodiments, the mGlu modulator is the mGlu₃-selective antagonist, beta-NAAG.

Additional non-limiting examples of reported glutamate modulators include memantine (CAS RN 19982-08-2), memantine hydrochloride (CAS RN 41100-52-1), and riluzole (CAS RN 1744-22-5).

In some non-limiting embodiments, a reported Group II modulator is administered in combination with one or more additional compounds reported as active against a Group I and/or a Group III mGlu receptor. For example, in some cases, methods comprise modulating the activity of at least one Group I receptor and at least one Group II mGlu receptor (e.g., with a compound described herein). Examples of compounds useful in modulating the activity of Group I receptors include Group I-selective agonists, such as (i) trans-azetidine-2,4,-dicarboxylic acid (tADA), which is described in Kozikowski et al., J. Med. Chem., 36: 2706 (1993) and Manahan-Vaughan et al., Neuroscience, 72: 999 (1996); (ii) (RS)-3,5-Dihydroxyphenylglycine (DHPG), which is described in Ito et al., NeuroReport 3: 1013 (1992); or a composition comprising (S)-DHPG substantially free of (R)-DHPG, as described, e.g., in Baker et al., Bioorg. Med. Chem. Lett. 5: 223 (1995); (iii) (RS)-3-Hydroxyphenylglycine, which is described in Birse et al., Neuroscience 52: 481 (1993); or a composition comprising (S)-3-Hydroxyphenylglycine substantially free of (R)-3-Hydroxyphenylglycine, as described, e.g., in Hayashi et al., J. Neurosci., 14: 3370 (1994); (iv) and (S)-Homoquisqualate, which is described in Porter et al., Br. J. Pharmacol., 106: 509 (1992).

Additional non-limiting examples of reported Group I modulators include (i) Group I agonists, such as (RS)-3,5-dihydroxyphenylglycine, described in Brabet et al., Neuropharmacology, 34, 895-903, 1995; and compounds described in U.S. Pat. Nos. 6,399,641 and 6,589,978, and US Pub No. 20030212066; (ii) Group I antagonists, such as (S)-4-Carboxy-3-hydroxyphenylglycine; 7-(Hydroxyimino)cyclopropa-β-chromen-1α-carboxylate ethyl ester; (RS)-1-Aminoindan-1,5-dicarboxylic acid (AIDA); 2-Methyl-6 (phenylethynyl)pyridine (MPEP); 2-Methyl-6-(2-phenylethenyl)pyridine (SIB-1893); 6-Methyl-2-(phenylazo)-3-pyridinol (SIB-1757); (Sa-Amino-4-carboxy-2-methylbenzeneacetic acid; and compounds described in U.S. Pat. Nos. 6,586,422, 5,783,575, 5,843,988, 5,536,721, 6,429,207, 5,696,148, and 6,218,385, and US Pub Nos. 20030109504, 20030013715, 20050154027, 20050004130, 20050209273, 20050197361, and 20040082592; (iii) mGlu₅-selective agonists, such as (RS)-2-Chloro-5-hydroxyphenylglycine (CHPG); and (iv) mGlu₅-selective antagonists, such as 2-methyl-6-(phenylethynyl)-pyridine (MPEP); and compounds described in U.S. Pat. No. 6,660,753; and US Pub Nos. 20030195139, 20040229917, 20050153986, 20050085514, 20050065340, 20050026963, 20050020585, and 20040259917.

Non-limiting examples of compounds reported to modulate Group III receptors include (i) the Group III-selective agonists (L)-2-amino-4-phosphonobutyric acid (L-AP4), described in Knopfel et al., J. Med Chem., 38, 1417-1426 (1995); and (S)-2-Amino-2-methyl-4-phosphonobutanoic acid; (ii) the Group III-selective antagonists (RS)-a-Cyclopropyl-4-phosphonophenylglycine; (RS)-α-Methylserine-O-phosphate (MSOP); and compounds described in US App. No. 20030109504; and (iii) (1S,3R,4S)-1-aminocyclopentane-1,2,4-tricarboxylic acid (ACPT-I).

In additional embodiments, the neurogenic agent used in combination with an HDac inhibitory agent may be a reported AMPA modulator. Non-limiting examples include CX-516 or ampalex (CAS RN 154235-83-3), Org-24448 (CAS RN 211735-76-1), LY451395 (2-propanesulfonamide, N-[(2R)-2-[4′-[2-[methylsulfonyl)amino]ethyl][1,1′-biphenyl]-4-yl]propyl]-), LY-450108 (see Jhee et al. “Multiple-dose plasma pharmacokinetic and safety study of LY450108 and LY451395 (AMPA receptor potentiators) and their concentration in cerebrospinal fluid in healthy human subjects.” J Clin Pharmacol. 2006 46(4):424-32), and CX717. Additional examples of reported antagonists include irampanel (CAS RN 206260-33-5) and E-2007.

Further non-limiting examples of reported AMPA receptor antagonists for use in combinations include YM90K (CAS RN 154164-30-4), YM872 or Zonampanel (CAS RN 210245-80-0), NBQX (or 2,3-Dioxo-6-nitro-7-sulfamoylbenzo(f)quinoxaline; CAS RN 118876-58-7), PNQX (1,4,7,8,9,10-hexahydro-9-methyl-6-nitropyrido[3,4-f]quinoxaline-2,3-dione), and ZK200775 ([1,2,3,4-tetrahydro-7-morpholinyl-2,3-dioxo-6-(fluoromethyl) quinoxalin-1-yl]methylphosphonate).

In additional embodiments, a neurogenic agent used in combination with an HDac inhibitory agent may be a reported muscarinic agent. Non-limiting examples of a reported muscarinic agent include a muscarinic agonist such as milameline (CI-979), or a structurally or functionally related compound disclosed in U.S. Pat. Nos. 4,786,648, 5,362,860, 5,424,301, 5,650,174, 4,710,508, 5,314,901, 5,356,914, or 5,356,912; or xanomeline, or a structurally or functionally related compound disclosed in U.S. Pat. Nos. 5,041,455, 5,043,345, or 5,260,314.

Other non-limiting examples include a muscarinic agent such as alvameline (LU 25-109), or a functionally or structurally compound disclosed in U.S. Pat. Nos. 6,297,262, 4,866,077, U.S. Pat. Nos. RE36,374, 4,925,858, PCT Publication No. WO 97/17074, or in Moltzen et al., J Med Chem. November 25, 1994;37(24):4085-99; 2,8-dimethyl-3-methylene-1-oxa-8-azaspiro[4.5]decane (YM-796) or YM-954, or a functionally or structurally related compound disclosed in U.S. Pat. No. 4,940,795, U.S. Pat. Nos. RE34,653, 4,996,210, 5,041,549, 5,403,931, or 5,412,096, or in Wanibuchi et al., Eur. J. Pharmacol., 187, 479-486 (1990); cevimeline (AF102B), or a functionally or structurally compound disclosed in U.S. Pat. Nos. 4,855,290, 5,340,821, 5,580,880 (American Home Products), or U.S. Pat. No. 4,981,858 (optical isomers of AF102B); sabcomeline (SB 202026), or a functionally or structurally related compound described in U.S. Pat. No. 5,278,170, U.S. Pat. Nos. RE35,593, 6,468,560, 5,773,619, 5,808,075, 5,545,740, 5,534,522, or 6,596,869, U.S. Patent Publication Nos. 2002/0127271, 2003/0129246, 2002/0150618, 2001/0018074, 2003/0157169, or 2001/0003588, Bromidge et al., J Med Chem. 19;40(26):4265-80 (1997), or Harries et al., British J. Pharm., 124, 409-415 (1998); talsaclidine (WAL 2014 FU), or a functionally or structurally compound disclosed in U.S. Pat. Nos. 5,451,587, 5,286,864, 5,508,405, 5,451,587, 5,286,864, 5,508,405, or 5,137,895, or in Pharmacol. Toxicol., 78, 59-68 (1996); or a 1-methyl-1,2,5,6-tetrahydropyridyl-1,2,5-thiadiazole derivative, such as tetra(ethyleneglycol)(4-methoxy-1,2,5-thiadiazol-3-yl)[3-(1-methyl-1,2,5,6-tetrahydropyrid-3-yl)-1,2,5-thiadiazol-4yl]ether or a compound that is functionally or structurally related to a 1-methyl-1,2,5,6-tetrahydropyridyl-1,2,5-thiadiazole derivative as provided by Cao et al. (“Synthesis and biological characterization of 1-methyl-1,2,5,6-tetrahydropyridyl-1,2,5-thiadiazole derivatives as muscarinic agonists for the treatment of neurological disorders.” J. Med. Chem. 46(20):4273-4286, 2003).

Yet additional non-limiting examples include besipiridine, SR-46559, L-689,660, S-9977-2, AF-102, thiopilocarpine, or an analog of clozapine, such as a pharmaceutically acceptable salt, ester, amide, or prodrug form thereof, or a diaryl[a,d]cycloheptene, such as an amino substituted form thereof, or N-desmethylclozapine, which has been reported to be a metabolite of clozapine, or an analog or related compound disclosed in US 2005/0192268 or WO 05/63254.

In other embodiments, the muscarinic agent is an ml receptor agonist selected from 55-LH-3B, 55-LH-25A, 55-LH-30B, 55-LH-4-1A, 40-LH-67, 55-LH-15A, 55-LH-16B, 55-LH-11C, 55-LH-31A, 55-LH-46, 55-LH-47, 55-LH-4-3A, or a compound that is functionally or structurally related to one or more of these agonists disclosed in US 2005/0130961 or WO 04/087158.

In additional embodiments, the muscarinic agent is a benzimidazolidinone derivative, or a functionally or structurally compound disclosed in U.S. Pat. No. 6,951,849, US 2003/0100545, WO 04/089942, or WO 03/028650; a spiroazacyclic compound, or a functionally or structurally related related compound like 1-oxa-3,8-diaza-spiro[4,5]decan-2-one or a compound disclosed in U.S. Pat. No. 6,911,452 or WO 03/057698; or a tetrahydroquinoline analog, or a functionally or structurally compound disclosed in US 2003/0176418, US 2005/0209226, or WO 03/057672.

In other embodiments, the neurogenic agent in combination with an HDac inhibitory agent is a reported GABA modulator which modulates GABA receptor activity at the receptor level (e.g., by binding directly to GABA receptors), at the transcriptional and/or translational level (e.g., by preventing GABA receptor gene expression), and/or by other modes (e.g., by binding to a ligand or effector of a GABA receptor, or by modulating the activity of an agent that directly or indirectly modulates GABA receptor activity). Non-limiting examples of GABA-A receptor modulators useful in methods described herein include triazolophthalazine derivatives, such as those disclosed in WO 99/25353, and WO/98/04560; tricyclic pyrazolo-pyridazinone analogues, such as those disclosed in WO 99/00391; fenamates, such as those disclosed in U.S. Pat. No. 5,637,617; triazolo-pyridazine derivatives, such as those disclosed in WO 99/37649, WO 99/37648, and WO 99/37644; pyrazolo-pyridine derivatives, such as those disclosed in WO 99/48892; nicotinic derivatives, such as those disclosed in WO 99/43661 and U.S. Pat. No. 5,723,462; muscimol, thiomuscimol, and compounds disclosed in U.S. Pat. No. 3,242,190; baclofen and compounds disclosed in U.S. Pat. No. 3,471,548; phaclofen; quisqualamine; ZAPA; zaleplon; THIP; imidazole-4-acetic acid (IMA); (+)-bicuculline; gabalinoleamide; isoguvicaine; 3-aminopropane sulphonic acid; piperidine-4-sulphonic acid; 4,5,6,7-tetrahydro-[5,4-c]-pyridin-3-ol; SR 95531; RU5315; CGP 55845; CGP 35348; FG 8094; SCH 50911; NG2-73; NGD-96-3; pricrotoxin and other bicyclophosphates disclosed in Bowery et al., Br. J. Pharmacol., 57; 435 (1976).

Additional non-limiting examples of GABA-A modulators include compounds described in U.S. Pat. Nos. 6,503,925; 6,218,547; 6,399,604; 6,646,124; 6,515,140; 6,451,809; 6,448,259; 6,448,246; 6,423,711; 6,414,147; 6,399,604; 6,380,209; 6,353,109; 6,297,256; 6,297,252; 6,268,496; 6,211,365; 6,166,203; 6,177,569; 6,194,427; 6,156,898; 6,143,760; 6,127,395; 6,103,903; 6,103,731; 6,723,735; 6,479,506; 6,476,030; 6,337,331; 6,730,676; 6,730,681; 6,828,322; 6,872,720; 6,699,859; 6,696,444; 6,617,326; 6,608,062; 6,579,875; 6,541,484; 6,500,828; 6,355,798; 6,333,336; 6,319,924; 6,303,605; 6,303,597; 6,291,460; 6,255,305; 6,133,255; 6,872,731; 6,900,215; 6,642,229; 6,593,325; 6,914,060; 6,914,063; 6,914,065; 6,936,608; 6,534,505; 6,426,343; 6,313,125 ; 6,310,203; 6,200,975; 6,071,909; 5,922,724; 6,096,887; 6,080,873; 6,013,799; 5,936,095; 5,925,770; 5,910,590; 5,908,932; 5,849,927; 5,840,888; 5,817,813; 5,804,686; 5,792,766; 5,750,702; 5,744,603; 5,744,602; 5,723,462; 5,696,260; 5,693,801; 5,677,309; 5,668,283; 5,637,725; 5,637,724; 5,625,063; 5,610,299; 5,608,079; 5,606,059; 5,604,235; 5,585,490; 5,510,480; 5,484,944; 5,473,073; 5,463,054; 5,451,585; 5,426,186; 5,367,077; 5,328,912 5,326,868; 5,312,822; 5,306,819; 5,286,860; 5,266,698; 5,243,049; 5,216,159; 5,212,310; 5,185,446; 5,185,446; 5,182,290; 5,130,430; 5,095,015; 20050014939; 20040171633; 20050165048; 20050165023; 20040259818; and 20040192692.

In some embodiments, the GABA-A modulator is a subunit-selective modulator. Non-limiting examples of GABA-A modulator having specificity for the alpha1 subunit include alpidem and zolpidem. Non-limiting examples of GABA-A modulator having specificity for the alpha2 and/or alpha3 subunits include compounds described in U.S. Pat. Nos. 6,730,681; 6,828,322; 6,872,720; 6,699,859; 6,696,444; 6,617,326; 6,608,062; 6,579,875; 6,541,484; 6,500,828; 6,355,798; 6,333,336; 6,319,924; 6,303,605; 6,303,597; 6,291,460; 6,255,305; 6,133,255; 6,900,215; 6,642,229; 6,593,325; and 6,914,063. Non-limiting examples of GABA-A modulator having specificity for the alpha2, alpha3 and/or alpha5 subunits include compounds described in U.S. Pat. Nos. 6,730,676 and 6,936,608. Non-limiting examples of GABA-A modulators having specificity for the alpha5 subunit include compounds described in U.S. Pat. Nos. 6,534,505; 6,426,343; 6,313,125 ; 6,310,203; 6,200,975 and 6,399,604. Additional non-limiting subunit selective GABA-A modulators include CL218,872 and related compounds disclosed in Squires et al., Pharmacol. Biochem. Behav., 10: 825 (1979); and beta-carboline-3-carboxylic acid esters described in Nielsen et al., Nature, 286: 606 (1980).

In some embodiments, the GABA-A receptor modulator is a reported allosteric modulator. In various embodiments, allosteric modulators modulate one or more aspects of the activity of GABA at the target GABA receptor, such as potency, maximal effect, affinity, and/or responsiveness to other GABA modulators. In some embodiments, allosteric modulators potentiate the effect of GABA (e.g., positive allosteric modulators), and/or reduce the effect of GABA (e.g., inverse agonists). Non-limiting examples of benzodiazepine GABA-A modulators include aiprazolam, bentazepam, bretazenil, bromazepam, brotizolam, cannazepam, chlordiazepoxide, clobazam, clonazepam, cinolazepam, clotiazepam, cloxazolam, clozapin, delorazepam, diazepam, dibenzepin, dipotassium chlorazepat, divaplon, estazolam, ethyl-loflazepat, etizolam, fludiazepam, flumazenil, flunitrazepam, flurazepaml 1HCl, flutoprazepam, halazeparn, haloxazolam, imidazenil, ketazolam, lorazepam, loprazolam, lormetazepam, medazepam, metaclazepam, mexozolam, midazolam-HCl, nabanezil, nimetazepam, nitrazepam, nordazepam, oxazepam-tazepam, oxazolam, pinazepam, prazepam, quazepam, sarmazenil, suriclone, temazepam, tetrazepam, tofisopam, triazolam, zaleplon, zolezepam, zolpidem, zopiclone, and zopielon.

Additional non-limiting examples of benzodiazepine GABA-A modulators include Ro15-4513, CL218872, CGS 8216, CGS 9895, PK 9084, U-93631, beta-CCM, beta-CCB, beta-CCP, Ro 19-8022, CGS 20625, NNC 14-0590, Ru 33-203, 5-amino-1-bromouracil, GYKI-52322, FG 8205, Ro 19-4603, ZG-63, RWJ46771, SX-3228, and L-655,078; NNC 14-0578, NNC 14-8198, and additional compounds described in Wong et al., Eur J Pharmacol 209: 319-325 (1995); Y-23684 and additional compounds in Yasumatsu et al., Br J Pharmacol 111: 1170-1178 (1994); and compounds described in U.S. Pat. No. 4,513,135.

Non-limiting examples of barbiturate or barbituric acid derivative GABA-A modulators include phenobarbital, pentobarbital, pentobarbitone, primidone, barbexaclon, dipropyl barbituric acid, eunarcon, hexobarbital, mephobarbital, methohexital, Na-methohexital, 2,4,6(1H,3H,5)-pyrimidintrion, secbutabarbital and/or thiopental.

Non-limiting examples of neurosteroid GABA-A modulators include alphaxalone, allotetrahydrodeoxycorticosterone, tetrahydrodeoxycorticosterone, estrogen, progesterone 3-beta-hydroxyandrost-5-en-17-on-3-sulfate, dehydroepianrosterone, eltanolone, ethinylestradiol, 5-pregnen-3-beta-ol-20 on-sulfate, 5a-pregnan-3a-ol-20-one (5PG), allopregnanolone, pregnanolone, and steroid derivatives and metabolites described in U.S. Pat. No. 5,939,545, 5,925,630, 6,277,838, 6,143,736, U.S. Pat. Nos. RE35,517, 5,925,630, 5,591,733, 5,232,917, 20050176976, WO 96116076, WO 98/05337, WO 95/21617, WO 94/27608, WO 93/18053, WO 93/05786, WO 93/03732, WO 91116897, EP01038880, and Han et al., J. Med. Chem., 36, 3956-3967 (1993), Anderson et al., J. Med. Chem., 40, 1668-1681 (1997), Hogenkamp et al., J. Med. Chem., 40, 61-72 (1997), Upasani et al., J. Med. Chem., 40, 73-84 (1997), Majewska et al., Science 232:1004-1007 (1986), Harrison et al., J. Pharmacol. Exp. Ther. 241:346-353 (1987), Gee et al., Eur. J. Pharmacol., 136:419-423 (1987) and Birtran et al., Brain Res., 561, 157-161 (1991).

Non-limiting examples of beta-carboline GABA-A modulators include abecarnil, 3,4-dihydro-beta-carboline, gedocarnil, 1-methyl-1-vinyl-2,3,4-trihydro-beta-carboline-3-carboxylic acid, 6-methoxy-1,2,3,4-tetrahydro-beta-carboline, N-BOC-L-1,2,3,4-tetrahydro-b-eta-carboline-3-carboxylic acid, tryptoline, pinoline, methoxyharmalan, tetrahydro-beta-carboline (THBC), 1-methyl-THBC, 6-methoxy-THBC, 6-hydroxy-THBC, 6-methoxyharmalan, norharman, 3,4-dihydro-beta-carboline, and compounds described in Nielsen et al., Nature, 286: 606 (1980).

In some embodiments, the GABA modulator modulates GABA-B receptor activity. Non-limiting examples of reported GABA-B receptor modulators useful in methods described herein include CGP36742; CGP-64213; CGP 56999A; CGP 54433A; CGP 36742; SCH 50911; CGP 7930; CGP 13501; baclofen and compounds disclosed in U.S. Pat. No. 3,471,548; saclofen; phaclofen; 2-hydroxysaclofen; SKF 97541; CGP 35348 and related compounds described in Olpe, et al, Eur. J. Pharmacol., 187, 27 (1990); phosphinic acid derivatives described in Hills, et al, Br. J. Pharmacol., 102, pp. 5-6 (1991); and compounds described in U.S. Pat. Nos. 4,656,298, 5,929,236, EP0463969, EP 0356128, Kaupmann et al., Nature 368: 239 (1997), Karla et al., J Med Chem., 42(11):2053-9 (1992), Ansar et al., Therapie, 54(5):651-8 (1999), and Castelli et al., Eur J Pharmacol., 446(1-3):1-5 (2002).

In some embodiments, the GABA modulator modulates GABA-C receptor activity. Non-limiting examples of reported GABA-C receptor modulators useful in methods described herein include cis-aminocrotonic acid (CACA); 1,2,5,6-tetrahydropyridine-4-yl methyl phosphinic acid (TPMPA) and related compounds such as P4MPA, PPA and SEPI; 2-methyl-TACA; (±)-TAMP; muscimol and compounds disclosed in U.S. Pat. No. 3,242,190; ZAPA; THIP and related analogues, such as aza-THIP; pricotroxin; imidazole-4-acetic acid (CIA); and CGP36742.

In some embodiments, the GABA modulator modulates the activity of glutamic acid decarboxylase (GAD).

In some embodiments, the GABA modulator modulates GABA transaminase (GTA). Non-limiting examples of GTA modulators include the GABA analogue vigabatrin and compounds disclosed in U.S. Pat. No. 3,960,927.

In some embodiments, the GABA modulator modulates the reuptake and/or transport of GABA from extracellular regions. In other embodiments, the GABA modulator modulates the activity of the GABA transporters, GAT-1, GAT-2, GAT-3 and/or BGT-1. Non-limiting examples of GABA reuptake and/or transport modulators include nipecotic acid and related derivatives, such as CI 966; SKF 89976A; TACA; stiripentol; tiagabine and GAT-1 inhibitors disclosed in U.S. Pat. No. 5,010,090; (R)-1-(4,4-diphenyl-3-butenyl)-3-piperidinecarboxylic acid and related compounds disclosed in U.S. Pat. No. 4,383,999; (R)-1-[4,4-bis(3-methyl-2-thienyl)-3-butenyl]-3-piperidinecarboxylic acid and related compounds disclosed in Anderson et al., J. Med. Chem. 36, (1993) 1716-1725; guvacine and related compounds disclosed in Krogsgaard-Larsen, Molecular & Cellular Biochemistry 31, 105-121 (1980); GAT-4 inhibitors disclosed in U.S. Pat. No. 6,071,932; and compounds disclosed in 6,906,177 and Ali, F. E., et al. J. Med. Chem. 1985, 28, 653-660. Methods for detecting GABA reuptake inhibitors are known in the art, and are described, e.g., in U.S. Pat. Nos. 6,906,177; 6,225,115; 4,383,999; Ali, F. E., et al. J. Med. Chem. 1985, 28, 653-660.

In some embodiments, the GABA modulator is the benzodiazepine Clonazepam, which is described, e.g., in U.S. Pat. Nos. 3,121,076 and 3,116,203; the benzodiazepine Diazepam, which is described, e.g., in U.S. Pat. Nos. 3,371,085; 3,109,843; and 3,136,815; the short-acting diazepam derivative Midazolam, which is a described, e.g., in U.S. Pat. No. 4,280,957; the imidazodiazepine Flumazenil, which is described, e.g., in U.S. Pat. No. 4,316,839; the benzodiazepine Lorazepam is described, e.g., in U.S. Pat. No. 3,296,249; the benzodiazepine L-655708, which is described, e.g., in Quirk et al. Neuropharmacolog 1996, 35, 133 1; Sur et al. Mol. Pharmacol. 1998, 54, 928; and Sur et al. Brain Res. 1999, 822, 265; the benzodiazepine Gabitril; Zopiclone, which binds the benzodiazepine site on GABA-A receptors, and is disclosed, e.g., in U.S. Pat. Nos. 3,862,149 and 4,220,646.; the GABA-A potentiator Indiplon as described, e.g., in Foster et al., J Pharmacol Exp Ther., 311(2):547-59 (2004), U.S. Pat. Nos. 4,521,422 and 4,900,836; Zolpidem, described, e.g., in U.S. Pat. No. 4,794,185 and EP50563; Zaleplon, described, e.g., in U.S. Pat. No. 4,626,538; Abecarnil, described, e.g., in Stephens et al., J Pharmacol Exp Ther., 253(1):334-43 (1990); the GABA-A agonist Isoguvacine, which is described, e.g., in Chebib et al., Clin. Exp. Pharmacol. Physiol. 1999, 26, 937-940; Leinekugel et al. J. Physiol. 1995, 487, 319-29; and White et al., J. Neurochem. 1983, 40(6), 1701-8; the GABA-A agonist Gaboxadol (THIP), which is described, e.g., in U.S. Pat. No. 4,278,676 and Krogsgaard-Larsen, Acta. Chem. Scand. 1977, 31, 584; the GABA-A agonist Muscimol, which is described, e.g., in U.S. Pat. Nos. 3,242,190 and 3,397,209; the inverse GABA-A agonist beta-CCP, which is described, e.g., in Nielsen et al., J. Neurochem., 36(1):276-85 (1981); the GABA-A potentiator Riluzole, which is described, e.g., in U.S. Pat. No. 4,370,338 and EP 50,55 1; the GABA-B agonist and GABA-C antagonist SKF 97541, which is described, e.g., in Froestl et al., J. Med. Chem. 38 3297 (1995); Hoskison et al., Neurosci. Lett. 2004, 365(1), 48-53 and Hue et al., J. Insect Physiol. 1997, 43(12), 1125-1131; the GABA-B agonist Baclofen, which is described, e.g., in U.S. Pat. No. 3,471,548; the GABA-C agonist cis-4-aminocrotonic acid (CACA), which is described, e.g., in Ulloor et al. J. Neurophysiol. 2004, 91(4), 1822-31; the GABA-A antagonist Phaclofen, which is described, e.g., in Kerr et al. Brain Res. 1987, 405, 150; Karlsson et al. Eur. J Pharmacol. 1988, 148, 485; and Hasuo, Gallagher Neurosci. Lett. 1988, 86, 77; the GABA-A antagonist SR 95531, which is described, e.g., in Stell et al. J. Neurosci. 2002, 22(10), RC223; Wermuth et al., J. Med. Chem. 30 239 (1987); and Luddens and Korpi, J. Neurosci. 15: 6957 (1995); the GABA-A antagonist Bicuculline, which is a described, e.g., in Groenewoud, J. Chem. Soc. 1936, 199; Olsen et al., Brain Res. 102: 283 (1976) and Haworth et al. Nature 1950, 165, 529; the selective GABA-B antagonist CGP 35348, which is described, e.g., in Olpe et al. Eur. J. Pharmacol. 1990, 187, 27; Hao et al. Neurosci. Lett. 1994, 182, 299; and Froestl et al. Pharmacol. Rev. Comm. 1996, 8, 127; the selective GABA-B antagonist CGP 46381, which is described, e.g., in Lingenhoehl, Pharmacol. Comm. 1993, 3, 49; the selective GABA-B antagonist CGP 52432, which is described, e.g., in Lanza et al. Eur. J. Pharmacol. 1993, 237, 191; Froestl et al. Pharmacol. Rev. Comm. 1996, 8, 127; Bonanno et al. Eur. J. Pharmacol. 1998, 362, 143; and Libri et al. Naunyn-Schmied. Arch. Pharmacol. 1998, 358, 168; the selective GABA-B antagonist CGP 54626, which is described, e.g., in Brugger et al. Eur. J. Pharmacol. 1993, 235, 153; Froestl et al. Pharmacol. Rev. Comm. 1996, 8, 127; and Kaupmann et al. Nature 1998, 396, 683; the selective GABA-B antagonist CGP 55845, which is a GABA-receptor antagonist described, e.g., in Davies et al. Neuropharmacology 1993, 32, 1071; Froestl et al. Pharmacol. Rev. Comm. 1996, 8, 127; and Deisz Neuroscience 1999, 93, 1241; the selective GABA-B antagonist Saclofen, which is described, e.g., in Bowery, TiPS, 1989, 10, 401; and Kerr et al. Neurosci Lett. 1988;92(1):92-6; the GABA-B antagonist 2-Hydroxysaclofen, which is described, e.g., in Kerr et al. Neurosci. Lett. 1988, 92, 92; and Curtis et al. Neurosci. Lett. 1988, 92, 97; the GABA-B antagonist SCH 50,911, which is described, e.g., in Carruthers et al., Bioorg Med Chem Lett 8: 3059-3064 (1998); Bolser et al. J. Pharmacol. Exp. Ther. 1996, 274, 1393; Hosford et al. J. Pharmacol. Exp. Ther. 1996, 274, 1399; and Ong et al. Eur. J. Pharmacol. 1998, 362, 35; the selective GABA-C antagonist TPMPA, which is described, e.g., in Schlicker et al., Brain Res. Bull. 2004, 63(2), 91-7; Murata et al., Bioorg. Med. Chem. Lett. 6: 2073 (1996); and Ragozzino et al., Mol. Pharmacol. 50: 1024 (1996); a GABA derivative, such as Pregabalin [(S)-(+)-3-isobutylgaba] or gabapentin [1-(aminomethyl)cyclohexane acetic acid]. Gabapentin is described, e.g., in U.S. Pat. No. 4,024,175; the lipid-soluble GABA agonist Progabide, which is metabolized in vivo into GABA and/or pharmaceutically active GABA derivatives in vivo. Progabide is described, e.g., in U.S. Pat. Nos. 4,094,992 and 4,361,583; the GAT1 inhibitor Tiagabine, which is described, e.g., in U.S. Pat. No. 5,010,090 and Andersen et al. J. Med. Chem. 1993, 36, 1716; the GABA transaminase inhibitor Valproic Acid (2-propylpentanoic acid or dispropylacetic acid), which is described, e.g., in U.S. Pat. No. 4,699,927 and Carraz et al., Therapie, 1965, 20, 419; the GABA transaminase inhibitor Vigabatrin, which is described, e.g., in U.S. Pat. No. 3,960,927; or Topiramate, which is described, e.g., in U.S. Pat. No. 4,513,006.

Additionally, the neurogenic agent in combination with an HDac inhibitory agent may be a neurogenic sensitizing agent that is a reported anti-epileptic agent. Non-limiting examples of such agents include carbamazepine or tegretol (CAS RN 298-46-4), clonazepam (CAS RN 1622-61-3), BPA or 3-(p-Boronophenyl)alanine (CAS RN 90580-64-6), gabapentin or neurontin (CAS RN 60142-96-3), phenytoin (CAS RN 57-41-0), topiramate, lamotrigine or lamictal (CAS RN 84057-84-1), phenobarbital (CAS RN 50-06-6), oxcarbazepine (CAS RN 28721-07-5), primidone (CAS RN 125-33-7), ethosuximide (CAS RN 77-67-8), levetiracetam (CAS RN 102767-28-2), zonisamide, tiagabine (CAS RN 115103-54-3), depakote or divalproex sodium (CAS RN 76584-70-8), Felbamate (Na-channel and NMDA receptor antagonist), or pregabalin (CAS RN 148553-50-8).

In further embodiments, the neurogenic sensitizing agent may be a reported direct or indirect modulator of dopamine receptors. Non-limiting examples of such agents include the indirect dopamine agonists methylphenidate (CAS RN 113-45-1) or Methylphenidate hydrochloride (also known as ritalin CAS RN 298-59-9), amphetamine (CAS RN 300-62-9) and methamphetamine (CAS RN 537-46-2), and the direct dopamine agonists sumanirole (CAS RN 179386-43-7), roprinirole (CAS RN 91374-21-9), and rotigotine (CAS RN 99755-59-6). Additional non-limiting examples include 7-OH-DPAT, quinpirole, haloperidole, or clozapine.

Additional non-limiting examples include bromocriptine (CAS RN 25614-03-3), adrogolide (CAS RN 171752-56-0), pramipexole (CAS RN 104632-26-0), Ropinirole (CAS RN 91374-21-9), apomorphine (CAS RN 58-00-4) or apomorphine hydrochloride (CAS RN 314-19-2), lisuride (CAS RN 18016-80-3), Sibenadet hydrochloride or Viozan (CAS RN 154189-24-9), L-DOPA or Levodopa (CAS RN 59-92-7), Melevodopa (CAS RN 7101-51-1), etilevodopa (CAS RN 37178-37-3), Talipexole hydrochloride (CAS RN 36085-73-1) or Talipexole (CAS RN 101626-70-4), Nolomirole (CAS RN 90060-42-7), quinelorane (CAS RN 97466-90-5), pergolide (CAS RN 66104-22-1), fenoldopam (CAS RN 67227-56-9), Carmoxirole (CAS RN 98323-83-2), terguride (CAS RN 37686-84-3), cabergoline (CAS RN 81409-90-7), quinagolide (CAS RN 87056-78-8) or quinagolide hydrochloride (CAS RN 94424-50-7), sumanirole, docarpamine (CAS RN 74639-40-0), SLV-308 or 2(3H)-Benzoxazolone, 7-(4-methyl-1-piperazinyl)-monohydrochloride (CAS RN 269718-83-4), aripiprazole (CAS RN 129722-12-9), bifeprunox, lisdexamfetamine dimesylate (CAS RN 608137-33-3), safinamide (CAS RN 133865-89-1), or Adderall or Amfetamine (CAS RN 300-62-9).

In further embodiments, the neurogenic agent used in combination with an HDac inhibitory agent may be a reported dual sodium and calcium channel modulator. Non-limiting examples of such agents include safinamide and zonisamide. Additional non-limiting examples include enecadin (CAS RN 259525-01-4), Levosemotiadil (CAS RN 116476-16-5), bisaramil (CAS RN 89194-77-4), SL-34.0829 (see U.S. Pat. No. 6,897,305), lifarizine (CAS RN 119514-66-8), JTV-519 (4-[3-(4-benzylpiperidin-1-yl)propionyl]-7-methoxy-2,3,4,5-tetrahydro-1,4-benzothiazepine monohydrochloride), and delapril.

In further embodiments, the neurogenic agent in used in combination with an HDac inhibitory agent may be a reported calcium channel antagonist such as amlodipine (CAS RN 88150-42-9) or amlodipine maleate (CAS RN 88150-47-4), nifedipine (CAS RN 21829-25-4), MEM-1003 (CAS RN see Rose et al. “Efficacy of MEM 1003, a novel calcium channel blocker, in delay and trace eyeblink conditioning in older rabbits.” Neurobiol Aging. Apr. 16, 2006; [Epub ahead of print]), isradipine (CAS RN 75695-93-1), felodipine (CAS RN 72509-76-3; 3,5-Pyridinedicarboxylic acid, 1,4-dihydro-4-(2,3-dichlorophenyl)-2,6-dimethyl-, ethyl methyl ester) or felodipine (CAS RN 86189-69-7; 3,5-Pyridinedicarboxylic acid, 4-(2,3-dichlorophenyl)-1,4-dihydro-2,6-dimethyl-, ethyl methyl ester, (±)-), lemildipine (CAS RN 125729-29-5 or 94739-29-4), clevidipine (CAS RN 166432-28-6 or 167221-71-8), verapamil (CAS RN 52-53-9), ziconotide (CAS RN 107452-89-1), monatepil maleate (CAS RN 132046-06-1), manidipine (CAS RN 89226-50-6), Furnidipine (CAS RN 138661-03-7), Nitrendipine (CAS RN 39562-70-4), Loperamide (CAS RN 53179-11-6), Amiodarone (CAS RN 1951-25-3), Bepridil (CAS RN 64706-54-3), diltiazem (CAS RN 42399-41-7), Nimodipine (CAS RN 66085-59-4), Lamotrigine, Cinnarizine (CAS RN 298-57-7), lacipidine (CAS RN 103890-78-4), nilvadipine (CAS RN 75530-68-6), dotarizine (CAS RN 84625-59-2), cilnidipine (CAS RN 132203-70-4), Oxodipine (CAS RN 90729-41-2), aranidipine (CAS RN 86780-90-7), anipamil (CAS RN 83200-10-6), ipenoxazone (CAS RN 104454-71-9), Efonidipine hydrochloride or NZ 105 (CAS RN 111011-53-1) or Efonidipine (CAS RN 111011-63-3), temiverine (CAS RN 173324-94-2), pranidipine (CAS RN 99522-79-9), dopropidil (CAS RN 79700-61-1), lercanidipine (CAS RN 100427-26-7), terodiline (CAS RN 15793-40-5), fantofarone (CAS RN 114432-13-2), azelnidipine (CAS RN 123524-52-7), mibefradil (CAS RN 116644-53-2) or mibefradil dihydrochloride (CAS RN 116666-63-8), SB-237376 (see Xu et al. “Electrophysiologic effects of SB-237376: a new antiarrhythmic compound with dual potassium and calcium channel blocking action.” J Cardiovasc Pharmacol. 2003 41(3):414-21), BRL-32872 (CAS RN 113241-47-7), S-2150 (see Ishibashi et al. “Pharmacodynamics of S-2150, a simultaneous calcium-blocking and alpha1-inhibiting antihypertensive drug, in rats.” J Pharm Pharmacol. 2000 52(3):273-80), nisoldipine (CAS RN 63675-72-9), semotiadil (CAS RN 116476-13-2), palonidipine (CAS RN 96515-73-0) or palonidipine hydrochloride (CAS RN 96515-74-1), SL-87.0495 (see U.S. Patent 6,897,305), YM430 (4(((S)-2-hydroxy-3-phenoxypropyl)amino)butyl methyl 2,6-dimethyl-((S)-4-(m-nitrophenyl))-1,4-dihydropyridine-3,5-dicarboxylate), barnidipine (CAS RN 104713-75-9), and AM336 or CVID (see Adams et al. “Omega-Conotoxin CVID Inhibits a Pharmacologically Distinct Voltage-sensitive Calcium Channel Associated with Transmitter Release from Preganglionic Nerve Terminals” J. Biol. Chem., 278(6):4057-4062, 2003). An additional non-limiting example is NMED-160.

In other embodiments, the neurogenic agent used in combination with an HDac inhibitory agent may be a reported modulator of a melatonin receptor. Non-limiting examples of such modulators include the melatonin receptor agonists melatonin, LY-156735 (CAS RN 118702-11-7), agomelatine (CAS RN 138112-76-2), 6-chloromelatonin (CAS RN 63762-74-3), Ramelteon (CAS RN 196597-26-9), 2-Methyl-6,7-dichloromelatonin (CAS RN 104513-29-3), and ML 23 (CAS RN 108929-03-9).

In yet further embodiments, the neurogenic agent in combination with an HDac inhibitory agent may be a reported modulator of a melanocortin receptor. Non-limiting examples of such agents include a melanocortin receptor agonists selected from melanotan II (CAS RN 121062-08-6), PT-141 or Bremelanotide (CAS RN 189691-06-3), HP-228 (see Getting et al. “The melanocortin peptide HP228 displays protective effects in acute models of inflammation and organ damage.” Eur J Pharmacol. Jan. 24, 2006), or AP214 from Action Pharma A/S.

Additional embodiments include a combination of an HDac inhibitory agent and a reported modulator of angiotensin II function, such as at an angiotensin II receptor. In some embodiments, the neurogenic sensitizing agent used with an HDac inhibitory agent may be a reported inhibitor of an angiotensin converting enzyme (ACE). Non-limiting examples of such reported inhibitors include a sulfhydryl-containing (or mercapto-containing) agent, such as Alacepril, captopril (Capoten®), fentiapril, pivopril, pivalopril, or zofenopril; a dicarboxylate-containing agent, such as enalapril (Vasotec® or Renitec®) or enalaprilat, ramipril (Altace® or Tritace® or Ramace®), quinapril (Accupril®) or quinapril hydrochloride, perindopril (Coversyl®) or perindopril erbumine (Aceon®), lisinopril (Lisodur® or Prinivil® or Zestril®); a phosphonate-containing (or phosphate-containing) agent, such as fosinopril (Monopril®), fosinoprilat, fosinopril sodium (CAS RN 88889-14-9), benazepril (Lotensin®) or benazepril hydrochloride, imidapril or imidapril hydrochloride, moexipril (Univasc®), or trandolapril (Mavik®). In other embodiments, a modulator is administered in the form of an ester that increases biovavailability upon oral administration with subsequent conversion into metabolites with greater activity.

Further embodiments include reported angiotensin II modulating entities that are naturally occurring, such as casokinins and lactokinins (breakdown products of casein and whey) which may be administered as such to obviate the need for their formation during digestion. Additional non-limiting embodiments of reported angiotensin receptor antagonists include candesartan (Atacand® or Ratacand®, 139481-59-7) or candesartan cilexetil; eprosartan (Teveten®) or eprosartan mesylate; irbesartan (Aprovel® or Karvea® or Avapro®); losartan (Cozaar® or Hyzaar®); olmesartan (Benicar®, CAS RN 144689-24-7) or olmesartan medoxomil (CAS RN 144689-63-4); telmisartan (Micardis® or Pritor®); or valsartan (Diovan®).

Additional non-limiting examples of a reported angiotensin modulator that may be used in a combination include nateglinide or starlix (CAS RN 105816-04-4); tasosartan or its metabolite enoltasosartan; omapatrilat (CAS RN 167305-00-2); or a a combination of nateglinide and valsartan, amoldipine and benazepril (Lotrel 10-40 or Lotrel 5-40), or delapril and manidipine (CHF 1521).

Additionally, the agent used with an HDac inhibitory agent may be a reported 5HT1a receptor agonist (or partial agonist) such as buspirone (buspar). In some embodiments, a reported 5HT1 a receptor agonist is an azapirone, such as, but not limited to, tandospirone, gepirone and ipsapirone. Non-limiting examples of additional reported 5HT1a receptor agonists include flesinoxan(CAS RN 98206-10-1), MDL 72832 hydrochloride, U-92016A, (+)-UH 301, F 13714, F 13640, 6-hydroxy-buspirone (see US 2005/0137206), S-6-hydroxy-buspirone (see US 2003/0022899), R-6-hydroxy-buspirone (see US 2003/0009851), adatanserin, buspirone-saccharide (see WO 00/12067) or 8-hydroxy-2-dipropylaminotetralin (8-OHDPAT).

Additional non-limiting examples of reported 5HT1a receptor agonists include OPC-14523 (1-[3-[4-(3-chlorophenyl)-1-piperazinyl]propyl]-5-methoxy-3,4-dihydro-2[1H]-quinolonone monomethanesulfonate); BMS-181100 or BMY 14802 (CAS RN 105565-56-8); flibanserin (CAS RN 167933-07-5); repinotan (CAS RN 144980-29-0); lesopitron (CAS RN 132449-46-8); piclozotan (CAS RN 182415-09-4); Aripiprazole, Org-13011 (1-(4-trifluoromethyl-2-pyridinyl)-4-[4-[2-oxo-1-pyrrolidinyl]butyl]piperazine (E)-2-butenedioate); SDZ-MAR-327 (see Christian et al. “Positron emission tomographic analysis of central dopamine D1 receptor binding in normal subjects treated with the atypical neuroleptic, SDZ MAR 327.” Int J Mol Med. 1998 1(1):243-7); MKC-242 ((S)-5-[3-[(1,4-benzodioxan-2-ylmethyl)amino]propoxy]-1,3-benzodioxole HCl); vilazodone; sarizotan (CAS RN 177975-08-5); roxindole (CAS RN 112192-04-8) or roxindole methanesulfonate (CAS RN 119742-13-1); alnespirone (CAS RN 138298-79-0); bromerguride (CAS RN 83455-48-5); xaliproden (CAS RN 135354-02-8); mazapertine succinate (CAS RN 134208-18-7) or mazapertine (CAS RN 134208-17-6); PRX-00023; F-13640 ((3-chloro-4-fluoro-phenyl)-[4-fluoro-4-[[(5-methyl-pyridin-2-ylmethyl)-amino]methyl]piperidin-1-yl]methanone, fumaric acid salt); eptapirone (CAS RN 179756-85-5); Ziprasidone (CAS RN 146939-27-7); Sunepitron (see Becker et al. “G protein-coupled receptors: In silico drug discovery in 3D” PNAS 2004 101(31):11304-11309); umespirone (CAS RN 107736-98-1); SLV-308; bifeprunox; and zalospirone (CAS RN 114298-18-9).

Yet further non-limiting examples include AP-521 (partial agonist from AsahiKasei) and Du-123015 (from Solvay).

Alternatively, the agent used with an HDac inhibitory agent may be a reported 5HT4 receptor agonist (or partial agonist). In some embodiments, a reported 5HT4 receptor agonist or partial agonist is a substituted benzamide, such as cisapride; individual, or a combination of, cisapride enantiomers ((+) cisapride and (−) cisapride); mosapride; and renzapride as non-limiting examples. In other embodiments, the chemical entity is a benzofuran derivative, such as prucalopride. Additional embodiments include indoles, such as tegaserod, or benzimidazolones. Other non-limiting chemical entities reported as a 5HT4 receptor agonist or partial agonist include zacopride (CAS RN 90182-92-6), SC-53116 (CAS RN 141196-99-8) and its racemate SC-49518 (CAS RN 146388-57-0), BIMU1 (CAS RN 127595-43-1), TS-951 (CAS RN 174486-39-6), or ML10302 CAS RN 148868-55-7). Additional non-limiting chemical entities include metoclopramide, 5-methoxytryptamine, RS67506, 2-[1-(4-piperonyl)piperazinyl]benzothiazole, RS66331, BIMU8, SB 205149 (the n-butyl quaternary analog of renzapride), or an indole carbazimidamide as described by Buchheit et al. (“The serotonin 5-HT4 receptor. 2. Structure-activity studies of the indole carbazimidamide class of agonists.” J Med Chem. (1995) 38(13):2331-8). Yet additional non-limiting examples include norcisapride (CAS RN 102671-04-5) which is the metabolite of cisapride; mosapride citrate; the maleate form of tegaserod (CAS RN 189188-57-6); zacopride hydrochloride (CAS RN 99617-34-2); mezacopride (CAS RN 89613-77-4); SK-951 ((±)-4-amino-N-(2-(1-azabicyclo(3.3.0)octan-5-yl)ethyl)-5-chloro-2,3-dihydro-2-methylbenzo(b)furan-7-carboxamide hemifumarate); ATI-7505, a cisapride analog from ARYx Therapeutics; SDZ-216-454, a selective 5HT4 receptor agonist that stimulates cAMP formation in a concentration dependent manner (see Markstein et al. “Pharmacological characterisation of 5-HT receptors positively coupled to adenylyl cyclase in the rat hippocampus.” Naunyn Schmiedebergs Arch Pharmacol. (1999) 359(6):454-9); SC-54750, or Aminomethylazaadamantane; Y-36912, or 4-amino-N-[1-[3-(benzylsulfonyl)propyl]piperidin-4-ylmethyl]-5-chloro-2-methoxybenzamide as disclosed by Sonda et al. (“Synthesis and pharmacological properties of benzamide derivatives as selective serotonin 4 receptor agonists.” Bioorg Med Chem. (2004) 12(10):2737-47); TKS159, or 4-amino-5-chloro-2-methoxy-N-[(2S,4S)-1-ethyl-2-hydroxymethyl-4-pyrrolidinyl]benzamide, as reported by Haga et al. (“Effect of TKS159, a novel 5-hydroxytryptamine4 agonist, on gastric contractile activity in conscious dogs.”; RS67333, or 1-(4-amino-5-chloro-2-methoxyphenyl)-3-(1-n-butyl-4-piperidinyl)-1-propanone; KDR-5169, or 4-amino-5-chloro-N-[1-(3-fluoro-4-methoxybenzyl)piperidin-4-yl]-2-(2-hydroxyethoxy)benzamide hydrochloride dihydrate as reported by Tazawa, et al. (2002) “KDR-5169, a new gastrointestinal prokinetic agent, enhances gastric contractile and emptying activities in dogs and rats.” Eur J Pharmacol 434(3): 169-76); SL65.0155, or 5-(8-amino-7-chloro-2,3-dihydro-1,4-benzodioxin-5-yl)-3-[1-(2-phenyl ethyl)-4-piperidinyl]-1,3,4-oxadiazol-2(3H)-one monohydrochloride; and Y-34959, or 4-Amino-5-chloro-2-methoxy-N-[1-[5-(1-methylindol-3-ylcarbonylamino)pentyl]piperidin-4-ylmethyl]benzamide.

Other non-limiting reported 5HT4 receptor agonists and partial agonists for use in combination with an HDac inhibitory agent include metoclopramide (CAS RN 364-62-5), 5-methoxytryptamine (CAS RN 608-07-1), RS67506 (CAS RN 168986-61-6), 2-[1-(4-piperonyl)piperazinyl]benzothiazole (CAS RN 155106-73-3), RS66331 (see Buccafusco et al. “Multiple Central Nervous System Targets for Eliciting Beneficial Effects on Memory and Cognition.” (2000) Pharmacology 295(2):438-446), BIMU8 (endo-N-8-methyl-8-azabicyclo[3.2.1]oct-3-yl)-2,3-dehydro-2-oxo-3-(prop-2-yl)-1 H-benzimid-azole-1-carboxamide), or SB 205149 (the n-butyl quaternary analog of renzapride). Compounds related to metoclopramide, such as metoclopramide dihydrochloride (CAS RN 2576-84-3) or metoclopramide dihydrochloride (CAS RN 5581-45-3) or metoclopramide hydrochloride (CAS RN 7232-21-5 or 54143-57-6) may also be used in a combination or method as described herein.

Additionally, the agent used with an HDac inhibitory agent may be a reported 5HT3 receptor antagonist such as azasetron (CAS RN 123039-99-6); Ondansetron (CAS RN 99614-02-5) or Ondansetron hydrochloride (CAS RN 99614-01-4); Cilansetron (CAS RN 120635-74-7); Aloxi or Palonosetron Hydrochloride (CAS RN 135729-62-3); Palenosetron (CAS RN 135729-61-2 or 135729-56-5); Cisplatin (CAS RN 15663-27-1); Lotronex or Alosetron hydrochloride (CAS RN 122852-69-1); Anzemet or Dolasetron mesylate (CAS RN 115956-13-3); zacopride or R-Zacopride; E-3620 ([3(S)-endo]-4-amino-5-chloro-N-(8-methyl-8-azabicyclo[3.2.1-]oct-3-yl-2[(1-methyl-2-butynyl)oxy]benzamide) or E-3620 HCl (3(S)-endo-4-amino-5-chloro-N-(8-methyl-8-azabicyclo [3.2.1]oct-3-yl)-2-(1-methyl-2-butinyl)oxy)-benzamide-HCl); YM 060 or Ramosetron hydrochloride (CAS RN 132907-72-3); a thieno[2,3-d]pyrimidine derivative antagonist described in U.S. Pat. No. 6,846,823, such as DDP 225 or MCI 225 (CAS RN 135991-48-9); Marinol or Dronabinol (CAS RN 1972-08-3); or Lac Hydrin or Ammonium lactate (CAS RN 515-98-0); Kytril or Granisetron hydrochloride (CAS RN 107007-99-8); Bemesetron (CAS RN 40796-97-2); Tropisetron (CAS RN 89565-68-4); Zatosetron (CAS RN 123482-22-4); Mirisetron (CAS RN 135905-89-4) or Mirisetron maleate (CAS RN 148611-75-0); or renzapride (CAS RN 112727-80-7).

Additionally, the agent used with an HDac inhibitory agent may be a reported 5HT2A/2C receptor antagonist such as Ketanserin (CAS RN 74050-98-9) or ketanserin tartrate; risperidone; olanzapine; adatanserin (CAS RN 127266-56-2); Ritanserin (CAS RN 87051-43-2); etoperidone; nefazodone; deramciclane (CAS RN 120444-71-5); Geoden or Ziprasidone hydrochloride (CAS RN 138982-67-9); Zeldox or Ziprasidone or Ziprasidone hydrochloride; EMD 281014 (7-[4-[2-(4-fluoro-phenyl)-ethyl]-piperazine-1-carbonyl]-1 H-indole-3-carbonitrile HCl); MDL 100907 or M100907 (CAS RN 139290-65-6); Effexor XR (Venlafaxine formulation); Zomaril or Iloperidone; quetiapine (CAS RN 111974-69-7) or Quetiapine fumarate (CAS RN 111974-72-2) or Seroquel; SB 228357 or SB 243213 (see Bromidge et al. “Biarylcarbamoylindolines are novel and selective 5-HT(2C) receptor inverse agonists: identification of 5-methyl-1-[[2-[(2-methyl-3-pyridyl)oxy]-5-pyridyl]carbamoyl]-6-trifluoromethylindoline (SB-243213) as a potential antidepressant/anxiolytic agent.” J Med Chem. 2000 43(6): 1123-34; SB 220453 or Tonabersat (CAS RN 175013-84-0); Sertindole (CAS RN 106516-24-9); Eplivanserin (CAS RN 130579-75-8) or Eplivanserin fumarate (CAS RN 130580-02-8); Lubazodone hydrochloride (CAS RN 161178-10-5); Cyproheptadine (CAS RN 129-03-3); Pizotyline or pizotifen (CAS RN 15574-96-6); Mesulergine (CAS RN 64795-35-3); Irindalone (CAS RN 96478-43-2); MDL 11939 (CAS RN 107703-78-6); or pruvanserin (CAS RN 443144-26-1).

Additional non-limiting examples of modulators include reported 5-HT2C agonists or partial agonists, such as m-chlorophenylpiperazine; or 5-HT2A receptor inverse agonists, such as ACP 103 (CAS RN: 868855-07-6), APD125 (from Arena Pharmaceuticals), AVE 8488 (from Sanofi-Aventis) or TGWOOAD/AA(from Fabre Kramer Pharmaceuticals).

Additionally, the agent used with an HDac inhibitory agent may be a reported 5HT6 receptor antagonist such as SB-357134 (N-(2,5-Dibromo-3-fluorophenyl)-4-methoxy-3-piperazin-1-ylbenzenesulfonamide); SB-271046 (5-chloro-N-(4-methoxy-3-(piperazin-1-yl)phenyl)-3-methylbenzo[b]thiophene-2-sulfonamide); Ro 04-06790 (N-(2,6-bis(methylamino)pyrimidin-4-yl)-4-aminobenzenesulfonamide); Ro 63-0563 (4-amino-N-(2,6 bis-methylamino-pyridin-4-yl)-benzene sulfonamide); clozapine or its metabolite N-desmethylclozapine; olanzapine (CAS RN 132539-06-1); fluperlapine (CAS RN 67121-76-0); seroquel (quetiapine or quetiapine fumarate); clomipramine (CAS RN 303-49-1); amitriptyline (CAS RN50-48-6); doxepin (CAS RN 1668-19-5); nortryptyline (CAS RN 72-69-5); 5-methoxytryptamine (CAS RN 608-07-1); bromocryptine (CAS RN 25614-03-3); octoclothepin (CAS RN 13448-22-1); chlorpromazine (CAS RN 50-53-3); loxapine (CAS RN 1977-10-2); fluphenazine (CAS RN 69-23-8); or GSK 742457 (presented by David Witty, “Early Optimisation of in vivo Activity: the discovery of 5-HT6 Receptor Antagonist 742457” GlaxoSmithKline at SCIpharm 2006, International Pharmaceutical Industry Conference in Edinburgh, 16 May 2006).

As an additional non-limiting example, the reported 5HT6 modulator may be SB-258585 (4-Iodo-N-[4-methoxy-3-(4-methyl-piperazin-1-yl)-phenyl]-benzen esulphonamide); PRX 07034 (from Predix Pharmaceuticals) or a partial agonist, such as E-6801 (6-chloro-N-(3-(2-(dimethylamino)ethyl)-1H-indol-5-yl)imidazo[2,1-b]thiazole-5-sulfonamide) or E-6837 (5-chloro-N-(3-(2-(dimethylamino)ethyl)-1H-indol-5-yl)naphthalene-2-sulfonamide).

Additionally, the agent used in combination with an HDac inhibitory agent may be a reported compound (or “monoamine modulator”) that modulates neurotransmission mediated by one or more monoamine neurotransmitters (referred to herein as “monoamines”) or other biogenic amines, such as trace amines (TAs) as a non-limiting example. TAs are endogenous, CNS-active amines that are structurally related to classical biogenic amines (e.g., norepinephrine, dopamine (4-(2-aminoethyl)benzene-1,2-diol), and/or serotonin (5-hydroxytryptamine (5-HT), or a metabolite, precursor, prodrug, or analogue thereof. The methods of the disclosure thus include administration of one or more reported TAs in a combination with an HDac inhibitory agent. Additional CNS-active monoamine receptor modulators are well known in the art, and are described, e.g., in the Merck Index, 12th Ed. (1996).

Certain food products, e.g., chocolates, cheeses, and wines, can also provide a significant dietary source of TAs and/or TA-related compounds. Non-limiting examples of mammalian TAs useful as constitutive factors include, but are not limited to, tryptamine, ρ-tyramine, m-tyramine, octopamine, synephrine or β-phenylethylamine (β-PEA). Additional useful TA-related compounds include, but are not limited to, 5-hydroxytryptamine, amphetamine, bufotenin, 5-methoxytryptamine, dihydromethoxytryptamine, phenylephrine, or a metabolite, precursor, prodrug, or analogue thereof.

In some embodiments, the constitutive factor is a biogenic amine or a ligand of a trace amine-associated receptor (TAAR), and/or an agent that mediates one or more biological effects of a TA. TAs have been shown to bind to and activate a number of unique receptors, termed TAARs, which comprise a family of G-protein coupled receptors (TAAR1-TAAR9) with homology to classical biogenic amine receptors. For example, TAAR1 is activated by both tyramine and β-PEA.

Thus non-limiting embodiments include methods and combination compositions wherein the constitutive factor is β-PEA, which has been indicated as having a significant neuromodulatory role in the mammalian CNS and is found at relatively high levels in the hippocampus (e.g., Taga et al., Biomed Chromatogr., 3(3): 118-20 (1989)); a metabolite, prodrug, precursor, or other analogue of β-PEA, such as the β-PEA precursor L-phenylalanine, the β-PEA metabolite β-phenylacetic acid (β-PAA), or the β-PEA analogues methylphenidate, amphetamine, and related compounds.

Most TAs and monoamines have a short half-life (e.g., less than about 30 s) due, e.g., to their rapid extracellular metabolism. Thus embodiments of the disclosure include use of a monoamine “metabolic modulator,” which increases the extracellular concentration of one or more monoamines by inhibiting monoamine metabolism. In some embodiments, the metabolic modulator is an inhibitor of the enzyme monoamine oxidase (MAO), which catalyzes the extracellular breakdown of monoamines into inactive species. Isoforms MAO-A and/or MAO-B provide the major pathway for TA metabolism. Thus, in some embodiments, TA levels are regulated by modulating the activity of MAO-A and/or MAO-B. For example, in some embodiments, endogenous TA levels are increased (and TA signaling is enhanced) by administering an inhibitor of MAO-A and/or MAO-B, in combination with an HDac inhibitory agent as described herein.

Non-limiting examples of inhibitors of monoamine oxidase (MAO) include reported inhibitors of the MAO-A isoform, which preferentially deaminates 5-hydroxytryptamine (serotonin) (5-HT) and norepinephrine (NE), and/or the MAO-B isoform, which preferentially deaminates phenylethylamine (PEA) and benzylamine (both MAO-A and MAO-B metabolize Dopamine (DA)). In various embodiments, MAO inhibitors may be irreversible or reversible (e.g., reversible inhibitors of MAO-A (RIMA)), and may have varying potencies against MAO-A and/or MAO-B (e.g., non-selective dual inhibitors or isoform-selective inhibitors). Non-limiting examples of MAO inhibitors useful in methods described herein include clorgyline, L-deprenyl, isocarboxazid (Marplan), ayahuasca, nialamide, iproniazide, iproclozide, moclobemide (Aurorix), phenelzine (Nardil), tranylcypromine (Pamate) (the congeneric of phenelzine), toloxatone, levo-deprenyl (Selegiline), harmala, RIMAs (e.g., moclobemide, described in Da Prada et al., J Pharmacol Exp Ther 248: 400-414 (1989); brofaromine; and befloxatone, described in Curet et al., J Affect Disord 51: 287-303 (1998)), lazabemide (Ro 19 6327), described in Ann. Neurol., 40(1): 99-107 (1996), and SL25.1131, described in Aubin et al., J. Pharmacol. Exp. Ther., 310: 1171-1182 (2004).

In additional embodiments, the monoamine modulator is an “uptake inhibitor,” which increases extracellular monoamine levels by inhibiting the transport of monoamines away from the synaptic cleft and/or other extracellular regions. In some embodiments, the monoamine modulator is a monoamine uptake inhibitor, which may selectively/preferentially inhibit uptake of one or more monoamines relative to one or more other monoamines. The term “uptake inhibitors” includes compounds that inhibit the transport of monoamines (e.g., uptake inhibitors) and/or the binding of monoamine substrates (e.g., uptake blockers) by transporter proteins (e.g., the dopamine transporter (DAT), the NE transporter (NET), the 5-HT transporter (SERT), and/or the extraneuronal monoamine transporter (EMT)) and/or other molecules that mediate the removal of extracellular monoamines. Monoamine uptake inhibitors are generally classified according to their potencies with respect to particular monoamines, as described, e.g., in Koe, J. Pharmacol. Exp. Ther. 199: 649-661 (1976). However, references to compounds as being active against one or more monoamines are not intended to be exhaustive or inclusive of the monoamines modulated in vivo, but rather as general guidance for the skilled practitioner in selecting compounds for use in therapeutic methods provided herein.

In embodiments relating to a biogenic amine modulator used in a combination or method with an HDac inhibitory agent as disclosed herein, the modulator may be (i) a norepinephrine and dopamine reuptake inhibitor, such as bupropion (described, e.g., in U.S. Pat. Nos. 3,819,706 and 3,885,046), or (S,S)-hydroxybupropion (described, e.g., in U.S. Pat. No. 6,342,496); (ii) selective dopamine reuptake inhibitors, such as medifoxamine, amineptine (described, e.g., in U.S. Pat. Nos. 3,758,528 and 3,821,249), GBR12909, GBR12783 and GBR13069, described in Andersen, Eur J Pharmacol, 166:493-504 (1989); or (iii) a monoamine “releaser” which stimulates the release of monoamines, such as biogenic amines from presynaptic sites, e.g., by modulating presynaptic receptors (e.g., autoreceptors, heteroreceptors), modulating the packaging (e.g., vesicular formation) and/or release (e.g., vesicular fusion and release) of monoamines, and/or otherwise modulating monoamine release. Advantageously, monoamine releasers provide a method for increasing levels of one or more monoamines within the synaptic cleft or other extracellular region independently of the activity of the presynaptic neuron.

Monoamine releasers useful in combinations provided herein include fenfluramine or p-chloroamphetamine (PCA) or the dopamine, norepinephrine, and serotonin releasing compound amineptine (described, e.g., in U.S. Pat. Nos. 3,758,528 and 3,821,249).

The agent used with an HDac inhibitory agent may be a reported phosphodiesterase (PDE) inhibitor. In some embodiments, a reported inhibitor of PDE activity include an inhibitor of a cAMP-specific PDE. Non-limiting examples of cAMP specific PDE inhibitors useful in the methods described herein include a pyrrolidinone, such as a compound disclosed in U.S. Pat. No. 5,665,754, US20040152754 or US20040023945; a quinazolineone, such as a compound disclosed in U.S. Pat. Nos. 6,747,035 or 6,828,315, WO 97/49702 or WO 97/42174; a xanthine derivative; a phenylpyridine, such as a compound disclosed in U.S. Pat. Nos. 6,410,547 or 6,090,817, or WO 97/22585; a diazepine derivative, such as a compound disclosed in WO 97/36905; an oxime derivative, such as a compound disclosed in U.S. Pat. No. 5,693,659 or WO 96/00215; a naphthyridine, such as a compound described in U.S. Pat. Nos. 5,817,670, 6,740,662, 6,136,821, 6,331,548, 6,297,248, 6,541,480, 6,642,250, or 6,900,205, or Trifilieff et al., Pharmacology, 301(1): 241-248 (2002), or Hersperger et al., J Med Chem., 43(4):675-82 (2000); a benzofuran, such as a compound disclosed in U.S. Pat. Nos. 5,902,824, 6,211,203, 6,514,996, 6,716,987, 6,376,535, 6,080,782, or 6,054,475, or EP 819688, EP685479, or Perrier et al., Bioorg. Med. Chem. Lett. 9:323-326 (1999); a phenanthridine, such as that disclosed in U.S. Pat. Nos. 6,191,138, 6,121,279, or 6,127,378; a benzoxazole, such as that disclosed in U.S. Pat. Nos. 6,166,041 or 6,376,485; a purine derivative, such as a compound disclosed in U.S. Pat. No. 6,228,859; a benzamide, such as a compound described in U.S. Pat. Nos. 5,981,527 or 5,712,298, or WO95/01338, WO 97/48697 or Ashton et al., J. Med Chem 37: 1696-1703 (1994); a substituted phenyl compound, such as a compound disclosed in U.S. Pat. Nos. 6,297,264, 5,866,593,65 5,859,034, 6,245,774, 6,197,792, 6,080,790, 6,077,854, 5,962,483, 5,674,880, 5,786,354, 5,739,144, 5,776,958, 5,798,373, 5,891,896, 5,849,770, 5,550,137, 5,340,827, 5,780,478, 5,780,477, or 5,633,257, or WO 95/35283; a substituted biphenyl compound, such as that disclosed in U.S. Pat. No. 5,877,190; or a quinilinone, such as a compound described in U.S. Pat. No. 6,800,625 or WO 98/14432.

Additional non-limiting examples of reported cAMP-specific PDE inhibitors useful in methods disclosed herein include a compound disclosed in U.S. Pat. Nos. 6,818,651, 6,737,436, 6,613,778, 6,617,357, 6,146,876, 6,838,559, 6,884,800, 6,716,987, 6,514,996, 6,376,535, 6,740,655, 6,559,168, 6,069,151, 6,365,585, 6,313,116, 6,245,774, 6,011,037, 6,127,363, 6,303,789, 6,316,472, 6,348,602, 6,331,543, 6,333,354, 5,491,147, 5,608,070, 5,622,977, 5,580,888, 6,680,336, 6,569,890, 6,569,885, 6,500,856, 6,486,186, 6,458,787, 6,455,562, 6,444,671, 6,423,710, 6,376,489, 6,372,777, 6,362,213, 6,313,156, 6,294,561, 6,258,843, 6,258,833, 6,121,279, 6,043,263, U.S. Pat. Nos. RE38,624, 6,297,257, 6,251,923, 6,613,794, 6,407,108, 6,107,295, 6,103,718, 6,479,494, 6,602,890, 6,545,158, 6,545,025, 6,498,160, 6,743,802, 6,787,554, 6,828,333, 6,869,945, 6,894,041, 6,924,292, 6,949,573, 6,953,810, 6,156,753, 5,972,927, 5,962,492, 5,814,651, 5,723,460, 5,716,967, 5,686,434, 5,502,072, 5,116,837, 5,091,431; 4,670,434; 4,490,371; 5,710,160, 5,710,170, 6,384,236, or 3,941,785, or US20050119225, US20050026913, US20050059686, US20040138279, US20050222138, US20040214843, US2004010663 1, US 20030045557, US 20020198198, US20030162802, US20030092908, US 20030104974, US20030100571, 20030092721, US20050148604, WO 99/65880, WO 00/26201, WO 98/06704, WO 00/59890, WO9907704, WO9422852, WO 98/20007, WO 02/096423, WO 98/18796, WO 98/02440, WO 02/096463, WO 97/44337, WO 97/44036, WO 97/44322, EP 0763534, Aoki et al., J Pharmacol Exp Ther., 295(1):255-60 (2000), Del Piaz et al., Eur. J. Med. Chem., 35; 463-480 (2000), or Barnette et al., Pharmacol. Rev. Commun. 8: 65-73 (1997).

In some embodiments, the reported cAMP-specific PDE inhibitor is Cilomilast (SB-207499); Filaminast; Tibenelast (LY-186655); Ibudilast; Piclamilast (RP 73401); Doxofylline; Cipamfylline (HEP-688); atizoram (CP-80633); theophylline; isobutylmethylxanthine; Mesopram (ZK-117137); Zardaverine; vinpocetine; Rolipram (ZK-62711); Arofylline (LAS-31025); roflumilast (BY-217); Pumafentrin (BY-343); Denbufylline; EHNA; milrinone; Siguazodan; Zaprinast; Tolafentrine; Isbufylline; IBMX; 1C-485; dyphylline; verolylline; bamifylline; pentoxyfilline; enprofilline; lirimilast (BAY 19-8004); filaminast (WAY-PDA-641); benafentrine; trequinsin; nitroquazone; cilostamide; vesnarinone; piroximone; enoximone; amrinone; olprinone; imazodan or 5-methyl-imazodan; indolidan; anagrelide; carbazeran; ampizone; emoradan; motapizone; phthalazinol; lixazinone (RS 82856); quazinone; bemorandan (RWJ 22867); adibendan (BM 14,478); Pimobendan (MCI-154); Saterinone (BDF 8634); Tetomilast (OPC-6535); benzafentrine; sulmazole (ARL 115); Revizinone; 349-U-85; AH-21-132; ATZ-1993; AWD-12-343; AWD-12-281; AWD-12-232; BRL 50481; CC-7085; CDC-801; CDC-998; CDP-840; CH-422; CH-673; CH-928; CH-3697; CH-3442; CH-2874; CH-4139; Chiroscience 245412; CI-930; CI-1018; CI-1044; CI-1118; CP-353164; CP-77059; CP-146523; CP-293321; CP-220629; CT-2450; CT-2820; CT-3883; CT-5210; D-4418; D-22888; E-4021; EMD 54622; EMD-53998; EMD-57033; GF-248; GW-3600; IC-485; ICI 63197; ICI 153,110; IPL-4088; KF-19514; KW-4490; L-787258; L-826141; L-791943; LY181512; NCS-613; NM-702; NSP-153; NSP-306; NSP-307; Org-30029; Org-20241; Org-9731; ORG 9935; PD-168787; PD-190749; PD-190036; PDB-093; PLX650; PLX369; PLX371; PLX788; PLX939; Ro-20-1724; RPR-132294; RPR-117658A; RPR-114597; RPR-122818; RPR-132703; RS-17597; RS-25344; RS-14203; SCA 40; Sch-351591; SDZ-ISQ-844; SDZ-MKS-492; SKF 94120; SKF-95654; SKF-107806; SKF 96231; T-440; T-2585; WAY-126120; WAY-122331; WAY-127093B; WIN-63291; WIN-62582; V-11294A; VMX 554; VMX 565; XT-044; XT-611; Y-590; YM-58897; YM-976; ZK-62711; methyl 3-[6-(2H-3,4,5,6-tetrahydropyran-2-yloxy)-2-(3-thienylcarbonyl)benzo[b]furan-3-yl]propanoate; 4-[4-methoxy-3-(5-phenylpentyloxy)phenyl]-2-methylbenzoic acid; methyl 3-{2-[(4-chlorophenyl)carbonyl]-6-hydroxybenzo[b]furan-3-yl}propanoate; (R*,R*)-(±)-methyl 3-acetyl-4-[3-(cyclopentyloxy)-4-methoxyphenyl]-3-methyl-1-pyrrolidinecarboxylat; or 4-(3-bromophenyl)-1-ethyl-7-methylhydropyridino[2,3-b]pyridin-2-one.

In some embodiments, the reported PDE inhibitor inhibits a cGMP-specific PDE. Non-limiting examples of a cGMP specific PDE inhibitor for use in the combinations and methods described herein include a pyrimidine or pyrimidinone derivative, such as a compound described in U.S. Pat. Nos. 6,677,335, 6,458,951, 6,251,904, 6,787,548, 5,294,612, 5,250,534, or 6,469,012, WO 94/28902, WO96/16657, EP0702555, and Eddahibi, Br. J. Pharmacol., 125(4): 681-688 (1988); a griseolic acid derivative, such as a compound disclosed in U.S. Pat. No. 4,460,765; a 1-arylnaphthalene lignan, such as that described in Ukita, J. Med. Chem. 42(7): 1293-1305 (1999); a quinazoline derivative, such as 4-[[3′,4′-(methylenedioxy)benzyl]amino]-6-methoxyquinazoline) or a compound described in U.S. Pat. Nos. 3,932,407 or 4,146,718, or U.S. Pat. No. RE31,617; a pyrroloquinolone or pyrrolopyridinone, such as that described in U.S. Pat. Nos. 6,686,349, 6,635,638, 6,818,646, US20050113402; a carboline derivative, such a compound described in U.S. Pat. Nos. 6,492,358, 6,462,047, 6,821,975, 6,306,870, 6,117,881, 6,043,252, or 3,819,631, US20030166641, WO 97/43287, Daugan et al., J Med Chem., 46(21):4533-42 (2003), or Daugan et al., J Med Chem., 9;46(21):4525-32 (2003); an imidazo derivative, such as a compound disclosed in U.S. Pat. Nos. 6,130,333, 6,566,360, 6,362,178, or 6,582,351, US20050070541, or US20040067945; or a compound described in U.S. Pat. Nos. 6,825,197, 5,719,283, 6,943,166, 5,981,527, 6,576,644, 5,859,009, 6,943,253, 6,864,253, 5,869,516, 5,488,055, 6,140,329, 5,859,006, or 6,143,777, WO 96/16644, WO 01/19802, WO 96/26940, Dunn, Org. Proc. Res. Dev., 9: 88-97 (2005), or Bi et al., Bioorg Med Chem Lett., 11(18):2461-4 (2001).

In some embodiments, the PDE inhibitor used in a combination or method disclosed herein is caffeine. In some embodiments, the caffeine is administered in a formulation comprising an HDac inhibitory agent. In other embodiments, the caffeine is administered simultaneously with an HDac inhibitory agent. In alternative embodiments, the caffeine is administered in a formulation, dosage, or concentration lower or higher than that of a caffeinated beverage such as coffee, tea, or soft drinks. In further embodiments, the caffeine is administered by a non-oral means, including, but not limited to, parenteral (e.g., intravenous, intradermal, subcutaneous, inhalation), transdermal (topical), transmucosal, rectal, or intranasal (including, but not limited to, inhalation of aerosol suspensions for delivery of compositions to the nasal mucosa, trachea and bronchioli) administration. The disclosure includes embodiments with the explicit exclusion of caffeine or another one or more of the described agents for use in combination with an HDac inhibitory agent.

In further alternative embodiments, the caffeine is in an isolated form, such as that which is separated from one or more molecules or macromolecules normally found with caffeine before use in a combination or method as disclosed herein. In other embodiments, the caffeine is completely or partially purified from one or more molecules or macromolecules normally found with the caffeine. Exemplary cases of molecules or macromolecules found with caffeine include a plant or plant part, an animal or animal part, and a food or beverage product.

Non-limiting examples of a reported PDE1 inhibitor include IBMX; vinpocetine; MMPX; KS-505a; SCH-51866; W-7; PLX650; PLX371; PLX788; a phenothiazines; or a compound described in U.S. Pat. No. 4,861,891.

Non-limiting examples of a PDE2 inhibitor include EHNA; PLX650; PLX369; PLX788; PLX 939; Bay 60-7550 or a related compound described in Boess et al., Neuropharmacology, 47(7):1081-92 (2004); or a compound described in US20020132754.

Non-limiting examples of reported PDE3 inhibitors include a dihydroquinolinone compound such as cilostamide, cilostazol, vesnarinone, or OPC 3911; an imidazolone such as piroximone or enoximone; a bipyridine such as milrinone, amrinone or olprinone; an imidazoline such as imazodan or 5-methyl-imazodan; a pyridazinone such as indolidan; LY181512 (see Komas et al. “Differential sensitivity to cardiotonic drugs of cyclic AMP phosphodiesterases isolated from canine ventricular and sinoatrial-enriched tissues.” J Cardiovasc Pharmacol. 1989 14(2):213-20); ibudilast; isomazole; motapizone; phthalazinol; trequinsin; lixazinone (RS 82856); Y-590; SKF 94120; quazinone; ICI 153,110; bemorandan (RWJ 22867); siguazodan (SK&F 94836); adibendan (BM 14,478); Pimobendan (UD-CG 115, MCI-154); Saterinone (BDF 8634); NSP-153; zardaverine; a quinazoline; benzafentrine; sulmazole (ARL 115); ORG 9935; CI-930; SKF-95654; SDZ-MKS-492; 349-U-85; EMD-53998; EMD-57033; NSP-306; NSP-307; Revizinone; NM-702; WIN-62582; ATZ-1993; WIN-63291; ZK-62711; PLX650; PLX369; PLX788; PLX939; anagrelide; carbazeran; ampizone; emoradan; or a compound disclosed in U.S. Pat. No. 6,156,753.

Non-limiting examples of reported PDE4 inhibitors include a pyrrolidinone, such as a compound disclosed in U.S. Pat. No. 5,665,754, US20040152754 or US20040023945; a quinazolineone, such as a compound disclosed in U.S. Pat. Nos. 6,747,035 or 6,828,315, WO 97/49702 or WO 97/42174; a xanthine derivative; a phenylpyridine, such as a compound disclosed in U.S. Pat. Nos. 6,410,547 or 6,090,817 or WO 97/22585; a diazepine derivative, such as a compound disclosed in WO 97/36905; an oxime derivative, such as a compound disclosed in U.S. Pat. No. 5,693,659 or WO 96/00215; a naphthyridine, such as a compound described in U.S. Pat. Nos. 5,817,670, 6,740,662, 6,136,821, 6,331,548, 6,297,248, 6,541,480, 6,642,250, or 6,900,205, Trifilieff et al., Pharmacology, 301(1): 241-248 (2002) or Hersperger et al., J Med Chem., 43(4):675-82 (2000); a benzofuran, such as a compound disclosed in U.S. Pat. Nos. 5,902,824, 6,211,203, 6,514,996, 6,716,987, 6,376,535, 6,080,782, or 6,054,475, EP 819688, EP685479, or Perrier et al., Bioorg. Med. Chem. Lett. 9:323-326 (1999); a phenanthridine, such as that disclosed in U.S. Pat. Nos. 6,191,138, 6,121,279, or 6,127,378; a benzoxazole, such as that disclosed in U.S. Pat. Nos. 6,166,041 or 6,376,485; a purine derivative, such as a compound disclosed in U.S. Pat. No. 6,228,859; a benzamide, such as a compound described in U.S. Pat. Nos. 5,981,527 or 5,712,298, WO95/01338, WO 97/48697, or Ashton et al., J. Med Chem 37: 1696-1703 (1994); a substituted phenyl compound, such as a compound disclosed in U.S. Pat. Nos. 6,297,264, 5,866,593,65 5,859,034, 6,245,774, 6,197,792, 6,080,790, 6,077,854, 5,962,483, 5,674,880, 5,786,354, 5,739,144, 5,776,958, 5,798,373, 5,891,896, 5,849,770, 5,550,137, 5,340,827, 5,780,478, 5,780,477, or 5,633,257, or WO 95/35283; a substituted biphenyl compound, such as that disclosed in U.S. Pat. No. 5,877,190; or a quinilinone, such as a compound described in U.S. Pat. No. 6,800,625 or WO 98/14432.

Additional examples of reported PDE4 inhibitors useful in methods provided herein include a compound disclosed in U.S. Pat. Nos. 6,716,987, 6,514,996, 6,376,535, 6,740,655, 6,559,168, 6,069,151, 6,365,585, 6,313,116, 6,245,774, 6,011,037, 6,127,363, 6,303,789, 6,316,472, 6,348,602, 6,331,543, 6,333,354, 5,491,147, 5,608,070, 5,622,977, 5,580,888, 6,680,336, 6,569,890, 6,569,885, 6,500,856, 6,486,186, 6,458,787, 6,455,562, 6,444,671, 6,423,710, 6,376,489, 6,372,777, 6,362,213, 6,313,156, 6,294,561, 6,258,843, 6,258,833, 6,121,279, 6,043,263, U.S. Pat. Nos. RE38,624, 6,297,257, 6,251,923, 6,613,794, 6,407,108, 6,107,295, 6,103,718, 6,479,494, 6,602,890, 6,545,158, 6,545,025, 6,498,160, 6,743,802, 6,787,554, 6,828,333, 6,869,945, 6,894,041, 6,924,292, 6,949,573, 6,953,810, 5,972,927, 5,962,492, 5,814,651, 5,723,460, 5,716,967, 5,686,434, 5,502,072, 5,116,837, 5,091,431; 4,670,434; 4,490,371; 5,710,160, 5,710,170, 6,384,236, or 3,941,785, US20050119225, US20050026913, WO 99/65880, WO 00/26201, WO 98/06704, WO 00/59890, WO9907704, WO9422852, WO 98/20007, WO 02/096423, WO 98/18796, WO 98/02440, WO 02/096463, WO 97/44337, WO 97/44036, WO 97/44322, EP 0763534, Aoki et al., J Pharmacol Exp Ther., 295(1):255-60 (2000), Del Piaz et al., Eur. J. Med. Chem., 35; 463-480 (2000), or Barnette et al., Pharmacol. Rev. Commun. 8: 65-73 (1997).

In some embodiments, the reported PDE4 inhibitor is Cilomilast (SB-207499); Filaminast; Tibenelast (LY-186655); Ibudilast; Piclamilast (RP 73401); Doxofylline; Cipamfylline (HEP-688); atizoram (CP-80633); theophylline; isobutylmethylxanthine; Mesopram (ZK-117137); Zardaverine; vinpocetine; Rolipram (ZK-627 11); Arofylline (LAS-31025); roflumilast (BY-217); Pumafentrin (BY-343); Denbufylline; EHNA; milrinone; Siguazodan; Zaprinast; Tolafentrine; Isbufylline; IBMX; IC-485; dyphylline; verolylline; bamifylline; pentoxyfilline; enprofilline; lirimilast (BAY 19-8004); filaminast (WAY-PDA-641); benafentrine; trequinsin; nitroquazone; Tetomilast (OPC-6535); AH-21-132; AWD-12-343; AWD-12-281; AWD-12-232; CC-7085; CDC-801; CDC-998; CDP-840; CH-422; CH-673; CH-928; CH-3697; CH-3442; CH-2874; CH-4139; Chiroscience 245412; CI-1018; CI-1044; CI-I118; CP-353164; CP-77059; CP-146523; CP-293321; CP-220629; CT-2450; CT-2820; CT-3883; CT-5210; D-4418; D-22888; E-4021; EMD 54622; GF-248; GW-3600; IC-485; ICI 63197; IPL-4088; KF-19514; KW-4490; L-787258; L-826141; L-791943; NCS-613; Org-30029; Org-20241; Org-9731; PD-168787; PD-190749; PD-190036; PDB-093; PLX650; PLX369; PLX371; PLX788; PLX939; Ro-20-1724; RPR-132294; RPR-117658A; RPR-114597; RPR-122818; RPR-132703; RS-17597; RS-25344; RS-14203; SCA 40; Sch-351591; SDZ-ISQ-844; SKF-107806; SKF 96231; T-440; T-2585; WAY-126120; WAY-122331; WAY-127093B; V-11294A;VMX 554; VMX 565; XT-044; XT-611; YM-58897; YM-976; methyl 3-[6-(2H-3,4,5,6-tetrahydropyran-2-yloxy)-2-(3-thienylcarbonyl)benzo[b]furan-3-yl]propanoate; 4-[4-methoxy-3-(5-phenylpentyloxy)phenyl]-2-methylbenzoic acid; methyl 3-{2-[(4-chlorophenyl)carbonyl]-6-hydroxybenzo[b]furan-3-yl}propanoate; (R*,R*)-(±)-methyl 3-acetyl-4-[3-(cyclopentyloxy)-4-methoxyphenyl]-3-methyl-1-pyrrolidinecarboxylat; or 4-(3-bromophenyl)-1-ethyl-7-methylhydropyridino[2,3-b]pyridin-2-one.

Non-limiting examples of a reported PDE5 inhibitor useful in a combination or method described herein include a pyrimidine or pyrimidinone derivative, such as a compound described in U.S. Pat. Nos. 6,677,335, 6,458,951, 6,251,904, 6,787,548, 5,294,612, 5,250,534, or 6,469,012, WO 94/28902, WO96/16657, EP0702555, or Eddahibi, Br. J. Pharmacol., 125(4): 681-688 (1988); a griseolic acid derivative, such as a compound disclosed in U.S. Pat. No. 4,460,765; a 1-arylnaphthalene lignan, such as that described in Ukita, J. Med. Chem. 42(7): 1293-1305 (1999); a quinazoline derivative, such as 4-[[3′,4′-(methylenedioxy)benzyl]amino]-6-methoxyquinazoline) or a compound described in U.S. Pat. Nos. 3,932,407 or 4,146,718, or U.S. Pat. No. RE31,617; a pyrroloquinolones or pyrrolopyridinone, such as that described in U.S. Pat. Nos. 6,686,349, 6,635,638, or 6,818,646, US20050113402; a carboline derivative, such a compound described in U.S. Pat. Nos. 6,492,358, 6,462,047, 6,821,975, 6,306,870, 6,117,881, 6,043,252, or 3,819,631, US20030166641, WO 97/43287, Daugan et al., J Med Chem., 46(21):4533-42 (2003), and Daugan et al., J Med Chem., 9;46(21):4525-32 (2003); an imidazo derivative, such as a compound disclosed in U.S. Pat. Nos. 6,130,333, 6,566,360, 6,362,178, or 6,582,351, US20050070541, or US20040067945; or a compound described in U.S. Pat. Nos. 6,825,197, 6,943,166, 5,981,527, 6,576,644, 5,859,009, 6,943,253, 6,864,253, 5,869,516, 5,488,055, 6,140,329, 5,859,006, or 6,143,777, WO 96/16644, WO 01/19802, WO 96/26940, Dunn, Org. Proc. Res. Dev., 9: 88-97 (2005), or Bi et al., Bioorg Med Chem Lett., 11(18):2461-4 (2001).

In some embodiments, a reported PDE5 inhibitor is zaprinast; MY-5445; dipyridamole; vinpocetine; FR229934; 1-methyl-3-isobutyl-8-(methylamino)xanthine; furazlocillin; Sch-51866; E4021; GF-196960; IC-351; T-1032; sildenafil; tadalafil; vardenafil; DMPPO; RX-RA-69; KT-734; SKF-96231; ER-21355; BF/GP-385; NM-702; PLX650; PLX134; PLX369; PLX788; or vesnarinone.

In some embodiments, the reported PDE5 inhibitor is sildenafil or a related compound disclosed in U.S. Pat. Nos. 5,346,901, 5,250,534, or 6,469,012; tadalafil or a related compound disclosed in U.S. Pat. Nos. 5,859,006, 6,140,329, 6,821,975, or 6,943,166; or vardenafil or a related compound disclosed in U.S. Pat. No. 6,362,178.

Non-limiting examples of a reported PDE6 inhibitor useful in a combination or method described herein include dipyridamole or zaprinast.

Non-limiting examples of a reported PDE7 inhibitor for use in the combinations and methods described herein include BRL 50481; PLX369; PLX788; or a compound described in U.S. Pat. Nos. 6,818,651; 6,737,436, 6,613,778, 6,617,357; 6,146,876, 6,838,559, or 6,884,800, US20050059686; US20040138279; US20050222138; US20040214843; US2004010663 1; US 20030045557; US 20020198198; US20030162802, US20030092908, US 20030104974; US20030100571; 20030092721; or US20050148604.

A non-limiting examples of a reported inhibitor of PDE8 activity is dipyridamole.

Non-limiting examples of a reported PDE9 inhibitor useful in a combination or method described herein include SCH-51866; IBMX; or BAY 73-6691.

Non-limiting examples of a PDE10 inhibitor include sildenafil; SCH-51866; papaverine; Zaprinast; Dipyridamole; E4021; Vinpocetine; EHNA; Milrinone; Rolipram; PLX107; or a compound described in U.S. Pat. No. 6,930,114, US20040138249, or US20040249148.

Non-limiting examples of a PDE11 inhibitor includes IC-351 or a related compound described in WO 9519978; E4021 or a related compound described in WO 9307124; UK-235,187 or a related compound described in EP 579496; PLX788; Zaprinast; Dipyridamole; or a compound described in US20040106631 or Maw et al., Bioorg Med Chem Lett. Apr. 17, 2003;13(8):1425-8.

In some embodiments, the reported PDE inhibitor is a compound described in U.S. Pat. Nos. 5,091,431, 5,081,242, 5,066,653, 5,010,086, 4,971,972, 4,963,561, 4,943,573, 4,906,628, 4,861,891, 4,775,674, 4,766,118, 4,761,416, 4,739,056, 4,721,784, 4,701,459, 4,670,434, 4,663,320, 4,642,345, 4,593,029, 4,564,619, 4,490,371, 4,489,078, 4,404,380, 4,370,328, 4,366,156, 4,298,734, 4,289,772, U.S. Pat. Nos. RE30,511, 4,188,391, 4,123,534, 4,107,309, 4,107,307, 4,096,257, 4,093,617, 4,051,236, or 4,036,840.

In some embodiments, the reported PDE inhibitor inhibits dual-specificity PDE. Non-limiting examples of a dual-specificity PDE inhibitor useful in a combination or method described herein include a cAMP-specific or cGMP-specific PDE inhibitor described herein; MMPX; KS-505a; W-7; a phenothiazine; Bay 60-7550 or a related compound described in Boess et al., Neuropharmacology, 47(7):1081-92 (2004); UK-235,187 or a related compound described in EP 579496; or a compound described in U.S. Pat. Nos. 6,930,114 or 4,861,891, US20020132754, US20040138249, US20040249148, US20040106631, WO 951997, or Maw et al., Bioorg Med Chem Lett. Apr. 17, 2003;13(8):1425-8.

In some embodiments, a reported PDE inhibitor exhibits dual-selectivity, being substantially more active against two PDE isozymes relative to other PDE isozymes. For example, in some embodiments, a reported PDE inhibitor is a dual PDE4/PDE7 inhibitor, such as a compound described in US20030104974; a dual PDE3/PDE4 inhibitor, such as zardaverine, tolafentrine, benafentrine, trequinsine, Org-30029, L-686398, SDZ-ISQ-844, Org-20241, EMD-54622, or a compound described in U.S. Pat. Nos. 5,521,187, or 6,306,869; or a dual PDE1/PDE4 inhibitor, such as KF19514 (5-phenyl-3-(3-pyridyl)methyl-3H-imidazo[4,5-c][1,8]naphthyridin-4 (5H)-one).

Furthermore, the neurogenic agent in combination with an HDac inhibitory agent may be a reported neurosteroid. Non-limiting examples of such a neurosteroid include pregnenolone and allopregnenalone.

Alternatively, the neurogenic sensitizing agent may be a reported non-steroidal anti-inflammatory drug (NSAID) or an anti-inflammatory mechanism targeting agent in general. Non-limiting examples of a reported NSAID include a cyclooxygenase inhibitor, such as indomethacin, ibuprofen, celecoxib, cofecoxib, naproxen, or aspirin. Additional non-limiting examples for use in combination with an HDac inhibitory agent include rofecoxib, meloxicam, piroxicam, valdecoxib, parecoxib, etoricoxib, etodolac, nimesulide, acemetacin, bufexamac, diflunisal, ethenzamide, etofenamate, flobufen, isoxicam, kebuzone, lonazolac, meclofenamic acid, metamizol, mofebutazone, niflumic acid, oxyphenbutazone, paracetamol, phenidine, propacetamol, propyphenazone, salicylamide, tenoxicam, tiaprofenic acid, oxaprozin, lornoxicam, nabumetone, minocycline, benorylate, aloxiprin, salsalate, flurbiprofen, ketoprofen, fenoprofen, fenbufen, benoxaprofen, suprofen, piroxicam, meloxicam, diclofenac, ketorolac, fenclofenac, sulindac, tolmetin, xyphenbutazone, phenylbutazone, feprazone, azapropazone, flufenamic acid or mefenamic acid. The disclosure includes use of the above NSAID agents in amounts that reduce or avoid side effects and/or complications seen with their individual use in higher amounts or concentrations.

In additional embodiments, the neurogenic agent in combination with an HDac inhibitory agent may be a reported agent for treating migraines. Non-limiting examples of such an agent include a triptan, such as almotriptan or almotriptan malate; naratriptan or naratriptan hydrochloride; rizatriptan or rizatriptan benzoate; sumatriptan or sumatriptan succinate; zolmatriptan or zolmitriptan, frovatriptan or frovatriptan succinate; or eletriptan or eletriptan hydrobromide. Embodiments of the disclosure may exclude combinations of triptans and an SSRI or SNRI that result in life threatening serotonin syndrome.

Other non-limiting examples include an ergot derivative, such as dihydroergotamine or dihydroergotamine mesylate, ergotamine or ergotamine tartrate; diclofenac or diclofenac potassium or diclofenac sodium; flurbiprofen; amitriptyline; nortriptyline; divalproex or divalproex sodium; propranolol or propranolol hydrochloride; verapamil; methysergide (CAS RN 361-37-5); metoclopramide; prochlorperazine (CAS RN 58-38-8); acetaminophen; topiramate; GW274150 ([2-[(1-iminoethyl) amino]ethyl]-L-homocysteine); or ganaxalone (CAS RN 38398-32-2).

Additional non-limiting examples include a COX-2 inhibitor, such as Celecoxib.

In other embodiments, the neurogenic agent in combination with an HDac inhibitory agent may be a reported modulator of a nuclear hormone receptor. Nuclear hormone receptors are activated via ligand interactions to regulate gene expression, in some cases as part of cell signaling pathways. Non-limiting examples of a reported modulator include a dihydrotestosterone agonist such as dihydrotestosterone; a 2-quinolone like LG121071 (4-ethyl-1,2,3,4-tetrahydro-6-(trifluoromethyl)-8-pyridono[5,6-g]-quinoline); a non-steroidal agonist or partial agonist compound described in U.S. Pat. No. 6,017,924; LGD2226 (see WO 01/16108, WO 01/16133, WO 01/16139, and Rosen et al. “Novel, non-steroidal, selective androgen receptor modulators (SARMs) with anabolic activity in bone and muscle and improved safety profile.” J Musculoskelet Neuronal Interact. 2002 2(3):222-4); or LGD2941 (from collaboration between Ligand Pharmaceuticals Inc. and TAP Pharmaceutical Products Inc.).

Additional non-limiting examples of a reported modulator include a selective androgen receptor modulator (SARM) such as andarine, ostarine, prostarin, or andromustine (all from GTx, Inc.); bicalutamide or a bicalutamide derivative such as GTx-007 (U.S. Pat. No. 6,492,554); or a SARM as described in U.S. Pat. No. 6,492,554.

Further non-limiting examples of a reported modulator include an androgen receptor antagonist such as cyproterone, bicalutamide, flutamide, or nilutamide; a 2-quinolone such as LG120907, represented by the following structure

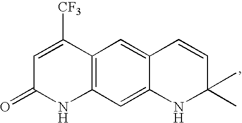

or a derivative compound represented by the following structure

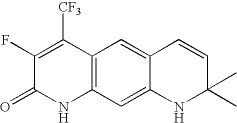

(see Allan et al. “Therapeutic androgen receptor ligands” Nucl Recept Signal 2003; 1: e009); a phthalamide, such as a modulator as described by Miyachi et al. (“Potent novel nonsteroidal androgen antagonists with a phthalimide skeleton.” Bioorg. Med. Chem. Lett. 1997 7:1483-1488); osaterone or osaterone acetate; hydroxyflutamide; or a non-steroidal antagonist described in U.S. Pat. No. 6,017,924.

Other non-limiting examples of a reported modulator include a retinoic acid receptor agonist such as all-trans retinoic acid (Tretinoin); isotretinoin (13-cis-retinoic acid); 9-cis retinoic acid; bexarotene; TAC-101 (4-[3,5-bis(trimethylsilyl)benzamide]benzoic acid); AC-261066 (see Lund et al. “Discovery of a potent, orally available, and isoform-selective retinoic acid beta2 receptor agonist.” J Med Chem. 2005 48(24):7517-9); LGD1 550 ((2E,4E,6E)-3-methyl-7-(3,5-di-ter-butylphen-yl)octatrienoic acid); E6060 (4-{5-[7-fluoro-4-(trifluoromethyl)benzo[b]furan-2-yl-1H-2-pyrrolyl}benzoic acid); agonist 1 or 2 as described by Schapira et al. (“In silico discovery of novel Retinoic Acid Receptor agonist structures.” BMC Struct Biol. 2001; 1: 1 (published online Jun. 4, 2001) where “Agonist 1 was purchased from Bionet Research (catalog number IG-433S). Agonist 2 was purchased from Sigma-Aldrich (Sigma Aldrich library of rare chemicals. Catalog number S08503-1”); a synthetic acetylenic retinoic acid, such as AGN 190121 (CAS RN: 132032-67-8), AGN 190168 (or Tazarotene or CAS RN 118292-40-3), or its metabolite AGN 190299 (CAS RN 118292-41-4); Etretinate; acitretin; an acetylenic retinoate, such as AGN 190073 (CAS 132032-68-9), or AGN 190089 (or 3-Pyridinecarboxylic acid, 6-(4-(2,6,6-trimethyl-1-cyclohexen-1-yl)-3-buten-1-ynyl)-, ethyl ester or CAS RN 116627-73-7).

In further embodiments, the additional agent for use in combination with an HDac inhibitory agent may be a reported modulator selected from thyroxin, tri-iodothyronine, or levothyroxine.

Alternatively, the additional agent is a vitamin D (1,25-dihydroxyvitamine D₃) receptor modulator, such as calcitriol or a compound described in Ma et al. (“Identification and characterization of noncalcemic, tissue-selective, nonsecosteroidal vitamin D receptor modulators.” J Clin Invest. 2006 116(4):892-904) or Molnar et al. (“Vitamin D receptor agonists specifically modulate the volume of the ligand-binding pocket.” J Biol Chem. 2006 281(15):10516-26) or Milliken et al. (“EB1089, a vitamin D receptor agonist, reduces proliferation and decreases tumor growth rate in a mouse model of hormone-induced mammary cancer.” Cancer Lett. 2005 229(2):205-15) or Yee et al. (“Vitamin D receptor modulators for inflammation and cancer.” Mini Rev Med Chem. 2005 5(8):761-78) or Adachi et al. “Selective activation of vitamin D receptor by lithocholic acid acetate, a bile acid derivative.” J Lipid Res. 2005 46(1):46-57).

Furthermore, the additional agent may be a reported cortisol receptor modulator, such as methylprednisolone or its prodrug methylprednisolone suleptanate; PI-1020 (NCX-1020 or budesonide-21-nitrooxymethylbenzoate); fluticasone furoate; GW-215864; betamethasone valerate; beclomethasone; prednisolone; or BVT-3498 (AMG-311).

Alternatively, the additional agent may be a reported aldosterone (or mineralocorticoid) receptor modulator, such as Spironolactone or Eplerenone.

In other embodiments, the additional agent may be a reported progesterone receptor modulator such as Asoprisnil (CAS RN 199396-76-4 ); mesoprogestin or J1042; J956; medroxyprogesterone acetate (MPA); R5020; tanaproget; trimegestone; progesterone; norgestomet; melengestrol acetate; mifepristone; onapristone; ZK137316; ZK230211 (see Fuhrmann et al. “Synthesis and biological activity of a novel, highly potent progesterone receptor antagonist.” J Med Chem. 2000 43(26):5010-6); or a compound described in Spitz “Progesterone antagonists and progesterone receptor modulators: an overview.” Steroids 2003 68(10-13):981-93.

In further embodiments, the additional agent may be a reported i) peroxisome proliferator-activated receptor agonist such as muraglitazar; tesaglitazar; reglitazar; GW-409544 (see Xu et al. “Structural determinants of ligand binding selectivity between the peroxisome proliferator-activated receptors.” Proc Natl Acad Sci USA. 2001 98(24):13919-24); or DRL 11605 (Dr. Reddy's Laboratories); ii) a peroxisome proliferator-activated receptor alpha agonist like clofibrate; ciprofibrate; fenofibrate; gemfibrozil; DRF-10945 (Dr. Reddy's Laboratories); iii) a peroxisome proliferator-activated receptor delta agonist such as GW501516 (CAS RN 317318-70-0); or iv) a peroxisome proliferator-activated gamma receptor agonist like a hydroxyoctadecadienoic acid (HODE); a prostaglandin derivatives, such as 15-deoxy-Delta12,14-prostaglandin J2; a thiazolidinedione (glitazone), such as pioglitazone, troglitazone; rosiglitazone or rosiglitazone maleate; ciglitazone; Balaglitazone or DRF-2593; AMG 131 (from Amgen); or G1262570 (from GlaxoWellcome).

In additional embodiments, the additional agent may be a reported modulator of an “orphan” nuclear hormone receptor. Embodiments include a reported modulator of a liver X receptor, such as a compound described in U.S. Pat. No. 6,924,311; a farnesoid X receptor, such as GW4064 as described by Maloney et al. (“Identification of a chemical tool for the orphan nuclear receptor FXR.” J Med Chem. 2000 43(16):2971-4); a RXR receptor; a CAR receptor, such as 1,4-bis[2-(3,5-dichloropyridyloxy)]benzene (TCPOBOP); or a PXR receptor, such as SR-12813 (tetra-ethyl 2-(3,5-di-tert-butyl-4-hydroxyphenyl)ethenyl-1,1-bisphosphonate).

In additional embodiments, the agent in combination with an HDac inhibitory agent is ethyl eicosapentaenoate or ethyl-EPA (also known as 5,8,11,14,17-eicosapentaenoic acid ethyl ester or miraxion, CAS RN 86227-47-6), docosahexaenoic acid (DHA), or a retinoid acid drug. As an additional non-limiting example, the agent may be Omacor, a combination of DHA and EPA, or idebenone (CAS RN 58186-27-9).

In further embodiments, a reported nootropic compound may be used as an agent in combination with an HDac inhibitory agent. Non-limiting examples of such a compound include Piracetam (Nootropil), Aniracetam, Oxiracetam, Pramiracetam, Pyritinol (Enerbol), Ergoloid mesylates (Hydergine), Galantamine or Galantamine hydrobromide, Selegiline, Centrophenoxine (Lucidril), Desmopressin (DDAVP), Nicergoline, Vinpocetine, Picamilon, Vasopressin, Milacemide, FK-960, FK-962, levetiracetam, nefiracetam, or hyperzine A (CAS RN: 102518-79-6).

Additional non-limiting examples of such a compound include anapsos (CAS RN 75919-65-2), nebracetam (CAS RN 97205-34-0 or 116041-13-5), metrifonate, ensaculin (or CAS RN 155773-59-4 or KA-672) or ensaculin HCl, Rokan (CAS RN 122933-57-7 or EGb 761), AC-3933 (5-(3-methoxyphenyl)-3-(5-methyl-1,2,4-oxadiazol-3-yl)-2-oxo-1,2-dihydro-1,6-naphthyridine) or its hydroxylated metabolite SX-5745 (3-(5-hydroxymethyl-1,2,4-oxadiazol-3-yl)-5-(3-methoxyphenyl)-2-oxo-1,2-dihydro-1,6-naphthyridine), JTP-2942 (CAS RN 148152-77-6), sabeluzole (CAS RN 104383-17-7), ladostigil (CAS RN 209394-27-4), choline alphoscerate (CAS RN 28319-77-9 or Gliatilin), Dimebon (CAS RN 3613-73-8), tramiprosate (CAS RN 3687-18-1), omigapil (CAS RN 181296-84-4), cebaracetam (CAS RN 113957-09-8), fasoracetam (CAS RN 110958-19-5), PD-151832 (see Jaen et al. “In vitro and in vivo evaluation of the subtype-selective muscarinic agonist PD 151832.” Life Sci. 1995 56(11-12):845-52), Vinconate (CAS RN 70704-03-9), PYM-50028 PYM-50028 (Cogane) or PYM-50018 (Myogane) as described by Harvey (“Natural Products in Drug Discovery and Development. 27-28 Jun. 2005, London, UK.” IDrugs. 2005 8(9):719-21), SR-46559A (3-[N-(2 diethyl-amino-2-methylpropyl)-6-phenyl-5-propyl), dihydroergocristine (CAS RN 17479-19-5), dabelotine (CAS RN 118976-38-8), zanapezil (CAS RN 142852-50-4).

Further non-limiting examples include NBI-113 (from Neurocrine Biosciences, Inc.), NDD-094 (from Novartis), P-58 or P58 (from Pfizer), or SR-57667 (from Sanofi-Synthelabo).

Moreover, an agent in combination with an HDac inhibitory agent may be a reported modulator of the nicotinic receptor. Non-limiting examples of such a modulator include nicotine, acetylcholine, carbamylcholine, epibatidine, ABT-418 (structurally similar to nicotine, with an ixoxazole moiety replacing the pyridyl group of nicotine), epiboxidine (a structural analogue with elements of both epibatidine and ABT-418), ABT-594 (azetidine analogue of epibatidine), lobeline, SSR-591813, represented by the following formula 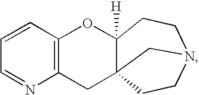
or SIB-1508 (altinicline).

In additional embodiments, an agent used in combination with an HDac inhibitory agent is a reported aromatase inhibitor. Reported aromatase inhibitors include, but are not limited to, nonsteroidal or steroidal agents. Non-limiting examples of the former, which inhibit aromatase via the heme prosthetic group, include anastrozole (Arimidex®), letrozole (Femara®), or vorozole (Rivisor). Non-limiting examples of steroidal aromatase inhibitors AIs, which inactivate aromatase, include, but are not limited to, exemestane (Aromasin®), androstenedione, or formestane (lentaron).

Additional non-limiting examples of a reported aromatase for use in a combination or method as disclosed herein include aminoglutethimide, 4-androstene-3,6,17-trione (or “6-OXO”), or zoledronic acid or Zometa (CAS RN 118072-93-8).

Further embodiments include a combination of an HDac inhibitory agent and a reported selective estrogen receptor modulator (SERM) may be used as described herein. Non-limiting examples include tamoxifen, raloxifene, toremifene, clomifene, bazedoxifene, arzoxifene, or lasofoxifene. Additional non-limiting examples include a steroid antagonist or partial agonist, such as centchroman, clomiphene, or droloxifene),

In other embodiments, a combination of an HDac inhibitory agent and a reported cannabinoid receptor modulator may be used as described herein. Non-limiting examples include synthetic cannabinoids, endogenous cannabinoids, or natural cannabinoids. In some embodiments, the reported cannabinoid receptor modulator is rimonabant (SR141716 or Acomplia), nabilone, levonantradol, marinol, or sativex (an extract containing both THC and CBD). Non-limiting examples of endogenous cannabinoids include arachidonyl ethanolamine (anandamide); analogs of anandamide, such as docosatetraenylethanolamide or homo-γ-linoenylethanolamide; N-acyl ethanolamine signalling lipids, such as the noncannabimimetic palmitoylethanolamine or oleoylethanolamine; or 2-arachidonyl glycerol. Non-limiting examples of natural cannabinoids include tetrahydrocannabinol (THC), cannabidiol (CBD), cannabinol (CBN), cannabigerol (CBG), cannabichromene (CBC), cannabicyclol (CBL), cannabivarol (CBV), tetrahydrocannabivarin (THCV), cannabidivarin (CBDV), cannabichromevarin (CBCV), cannabigerovarin (CBGV), or cannabigerol monoethyl ether (CBGM).

In yet further embodiments, an agent used in combination with an HDac inhibitory agent is a reported FAAH (fatty acid amide hydrolase) inhibitor. Non-limiting examples of reported inhibitor agents include URB597 (3′-carbamoyl-biphenyl-3-yl-cyclohexylcarbamate); CAY10401 (1-oxazolo[4,5-b]pyridin-2-yl-9-octadecyn-1-one); OL-135 (1-oxo-1[5-(2-pyridyl)-2-yl]-7-phenylheptane); anandamide (CAS RN 94421-68-8); AA-5-HT (see Bisogno et al. “Arachidonoylserotonin and other novel inhibitors of fatty acid amide hydrolase.” Biochem Biophys Res Commun. 1998 248(3):515-22); 1-Octanesulfonyl fluoride; or O-2142 or another arvanil derivative FAAH inhibitor as described by Di Marzo et al. (“A structure/activity relationship study on arvanil, an endocannabinoid and vanilloid hybrid.” J Pharmacol Exp Ther. 2002 300(3):984-91).

Further non-limiting examples include SSR 411298 (from Sanofi-Aventis), JNJ28614118 (from Johnson & Johnson), or SSR 101010 (from Sanofi-Aventis)

In additional embodiments, an agent in combination with an HDac inhibitory agent may be a reported modulator of nitric oxide function. One non-limiting example is sildenafil (Viagra®).

In additional embodiments, an agent in combination with an HDac inhibitory agent may be a reported modulator of prolactin or a prolactin modulator.

In additional embodiments, an agent in combination with an HDac inhibitory agent is a reported anti-viral agent, with ribavirin and amantadine as non-limiting examples.

In additional embodiments, an agent in combination with an HDac inhibitory agent may be a component of a natural product or a derivative of such a component. In some embodiments, the component or derivative thereof is in an isolated form, such as that which is separated from one or more molecules or macromolecules normally found with the component or derivative before use in a combination or method as disclosed herein. In other embodiments, the component or derivative is completely or partially purified from one or more molecules or macromolecules normally found with the component or derivative. Exemplary cases of molecules or macromolecules found with a component or derivative as described herein include a plant or plant part, an animal or animal part, and a food or beverage product.

Non-limiting examples such a component include folic acid; a flavinoid, such as a citrus flavonoid; a flavonol, such as Quercetin, Kaempferol, Myricetin, or Isorhamnetin; a flavone, such as Luteolin or Apigenin; a flavanone, such as Hesperetin, Naringenin, or Eriodictyol; a flavan-3-ol (including a monomeric, dimeric, or polymeric flavanol), such as (+)-Catechin, (+)-Gallocatechin, (−)-Epicatechin, (−)-Epigallocatechin, (−)-Epicatechin 3-gallate, (−)-Epigallocatechin 3-gallate, Theaflavin, Theaflavin 3-gallate, Theaflavin 3′-gallate, Theaflavin 3,3′ digallate, a Thearubigin, or Proanthocyanidin; an anthocyanidin, such as Cyanidin, Delphinidin, Malvidin, Pelargonidin, Peonidin, or Petunidin; an isoflavone, such as daidzein, genistein, or glycitein; flavopiridol; a prenylated chalcone, such as Xanthohumol; a prenylated flavanone, such as Isoxanthohumol; a non-prenylated chalcone, such as Chalconaringenin; a non-prenylated flavanone, such as Naringenin; Resveratrol; or an anti-oxidant neutraceutical (such as any present in chocolate, like dark chocolate or unprocessed or unrefined chocolate).

Additional non-limiting examples include a component of Gingko biloba, such as a flavo glycoside or a terpene. In some embodiments, the component is a flavanoid, such as a flavonol or flavone glycoside, or a quercetin or kaempferol glycoside, or rutin; or a terpenoid, such as ginkgolides A, B, C, or M, or bilobalide.

Further non-limiting examples include a component that is a flavanol, or a related oligomer, or a polyphenol as described in US2005/245601AA, US2002/018807AA, US2003/180406AA, US2002/086833AA, US2004/0236123, WO9809533, or WO9945788; a procyanidin or derivative thereof or polyphenol as described in US2005/171029AA; a procyanidin, optionally in combination with L-arginine as described in US2003/104075AA; a low fat cocoa extract as described in US2005/031762AA; lipophilic bioactive compound containing composition as described in US2002/107292AA; a cocoa extract, such as those containing one or more polyphenols or procyanidins as described in US2002/004523AA; an extract of oxidized tea leaves as described in U.S. Pat. Nos. 5,139,802 or 5,130,154; a food supplement as described in WO 2002/024002.

Of course a composition comprising any of the above components, alone or in combination with an HDac inhibitory agent as described herein is included within the disclosure.

In additional embodiments, an agent in combination with an HDac inhibitory agent may be a reported calcitonin receptor agonist such as calcitonin or the ‘orphan peptide’ PHM-27 (see Ma et al. “Discovery of novel peptide/receptor interactions: identification of PHM-27 as a potent agonist of the human calcitonin receptor.” Biochem Pharmacol. 2004 67(7): 1279-84). A further non-limiting example is the agonist from Kemia, Inc.

In an alternative embodiment, the agent may be a reported modulator of paratbyroid hormone activity, such as parathyroid hormone, or a modulator of the parathyroid hormone receptor.

In additional embodiments, an agent in combination with an HDac inhibitory agent may a reported antioxidant, such as N-acetylcysteine or acetylcysteine; disufenton sodium (or CAS RN 168021-79-2 or Cerovive); activin (CAS RN 104625-48-1); selenium; L-methionine; an alpha, gamma, beta, or delta, or mixed, tocopherol; alpha lipoic acid; Coenzyme Q; Benzimidazole; benzoic acid; dipyridamole; glucosamine; IRFI-016 (2(2,3-dihydro-5-acetoxy-4,6,7-trimethylbenzofuranyl)acetic acid); L-carnosine; L-Histidine; glycine; flavocoxid (or LIMBREL); baicalin, optionally with catechin (3,3′,4′,5,7-pentahydroxyflavan (2R,3S form)), and/or its stereo-isomer; masoprocol (CAS RN 27686-84-6); mesna (CAS RN 19767-45-4); probucol (CAS RN 23288-49-5); silibinin (CAS RN 22888-70-6); sorbinil (CAS RN 68367-52-2); spermine; tangeretin (CAS RN 481-53-8); butylated hydroxyanisole (BHA); butylated hydroxytoluene (BHT); propyl gallate (PG); tertiary-butyl-hydroquinone (TBHQ); nordihydroguaiaretic acid (CAS RN 500-38-9); astaxanthin (CAS RN 472-61-7); or an antioxidant flavonoid.

Additional non-limiting examples include a vitamin, such as vitamin A (Retinol) or C (Ascorbic acid) or E (including Tocotrienol and/or Tocopherol); a vitamin cofactors or mineral, such as Coenzyme Q10 (CoQ10), Manganese, or Melatonin; a carotenoid terpenoid, such as Lycopene, Lutein, Alpha-carotene, Beta-carotene, Zeaxanthin, Astaxanthin, or Canthaxantin; a non-carotenoid terpenoid, such as Eugenol; a flavonoid polyphenolic (or bioflavonoid); a flavonol, such as Resveratrol, Pterostilbene (methoxylated analogue of resveratrol), Kaempferol, Myricetin, Isorhamnetin, a Proanthocyanidin, or a tannin; a flavone, such as Quercetin, rutin, Luteol in, Apigenin, or Tangeritin; a flavanone, such as Hesperetin or its metabolite hesperidin, naringenin or its precursor naringin, or Eriodictyol; a flavan-3-ols (anthocyanidins), such as Catechin, Gallocatechin, Epicatechin or a gallate form thereof, Epigallocatechin or a gallate form thereof, Theaflavin or a gallate form thereof, or a Thearubigin; an isoflavone phytoestrogens, such as Genistein, Daidzein, or Glycitein; an anthocyanins, such as Cyanidin, Delphinidin, Malvidin, Pelargonidin, Peonidin, or Petunidin; a phenolic acid or ester thereof, such as Ellagic acid, Gallic acid, Salicylic acid, Rosmarinic acid, Cinnamic acid or a derivative thereof like ferulic acid, Chlorogenic acid, Chicoric acid, a Gallotannin, or an Ellagitannin; a nonflavonoid phenolic, such as Curcumin; an anthoxanthin, betacyanin, Citric acid, Uric acid, R-α-lipoic acid, or Silymarin.

Further non-limiting examples include 1-(carboxymethylthio)tetradecane; 2,2,5,7,8-pentamethyl-1-hydroxychroman; 2,2,6,6-tetramethyl-4-piperidinol-N-oxyl; 2,5-di-tert-butylhydroquinone; 2-tert-butylhydroquinone; 3,4-dihydroxyphenylethanol; 3-hydroxypyridine; 3-hydroxytamoxifen; 4-coumaric acid; 4-hydroxyanisole; 4-hydroxyphenylethanol; 4-methylcatechol; 5,6,7,8-tetrahydrobiopterin; 6,6′-methylenebis(2,2-dimethyl-4-methanesulfonic acid-1,2-dihydroquinoline); 6-hydroxy-2,5,7,8-tetramethylchroman-2-carboxylic acid; 6-methyl-2-ethyl-3-hydroxypyridine; 6-O-palmitoylascorbic acid; acetovanillone; acteoside; Actovegin; allicin; allyl sulfide; alpha-pentyl-3-(2-quinolinylmethoxy)benzenemethanol; alpha-tocopherol acetate; apolipoprotein A-IV; bemethyl; boldine; bucillamine; Calcium Citrate; Canthaxanthin; crocetin; diallyl trisulfide; dicarbine; dihydrolipoic acid; dimephosphon; ebselen; Efamol; enkephalin-Leu, Ala(2)-Arg(6)-; Ergothioneine; esculetin; essential 303 forte; Ethonium; etofyllinclofibrate; fenozan; glaucine; H290-51; histidyl-proline diketopiperazine; hydroquinone; hypotaurine; idebenone; indole-3-carbinol; isoascorbic acid; kojic acid, lacidipine, lodoxamide tromethamine; mexidol; morin; N,N′-diphenyl-4-phenylenediamine; N-isopropyl-N-phenyl-4-phenylenediamine; N-monoacetylcystine; nicaraven, nicotinoyl-GABA; nitecapone; nitroxyl; nobiletin; oxymethacil; p-tert-butyl catechol; phenidone; pramipexol; proanthocyanidin; procyanidin; prolinedithiocarbamate; Propyl Gallate; purpurogallin; pyrrolidine dithiocarbamic acid; rebamipide; retinol palmitate; salvin; Selenious Acid; sesamin; sesamol; sodium selenate; sodium thiosulfate; theaflavin; thiazolidine-4-carboxylic acid; tirilazad; tocopherylquinone; tocotrienol, alpha; a Tocotrienol; tricyclodecane-9-yl-xanthogenate; turmeric extract; U 74389F; U 74500A; U 78517F; ubiquinone 9; vanillin; vinpocetine; xylometazoline; zeta Carotene; zilascorb; zinc thionein; or zonisamide.

In additional embodiments, an agent in combination with an HDac inhibitory agent may be a reported modulator of a norepinephrine receptor. Non-limiting examples include Atomoxetine (Strattera); a norepinephrine reuptake inhibitor, such as talsupram, tomoxetine, nortriptyline, nisoxetine, reboxetine (described, e.g., in U.S. Pat. No. 4,229,449), or tomoxetine (described, e.g., in U.S. Pat. No. 4,314,081); or a direct agonist, such as a beta adrenergic agonist.

Non-limiting examples of reported adrenergic agonists include albuterol, albuterol sulfate, salbutamol (CAS RN 35763-26-9), clenbuterol, adrafinil, and SR58611A (described in Simiand et al., Eur J Pharmacol, 219:193-201 (1992)), clonidine (CAS RN 4205-90-7), yohimbine (CAS RN 146-48-5) or yohimbine hydrochloride, arbutamine; befunolol; BRL 26830A; BRL 35135; BRL 37344; bromoacetylalprenololmenthane; broxaterol; carvedilol; CGP 12177; cimaterol; cirazoline; CL 316243; Clenbuterol; denopamine; dexmedetomidine or dexmedetomidine hydrochloride; Dobutamine, dopexamine, Ephedrine, Epinephrine, Etilefrine; Fenoterol; formoterol; formoterol fumarate; Hexoprenaline; higenamine; ICI D7114; Isoetharine; Isoproterenol; Isoxsuprine; levalbuterol tartrate hydrofluoroalkane; lidamidine; mabuterol; methoxyphenamine; modafinil; Nylidrin; Orciprenaline; Oxyfedrine; pirbuterol; Prenalterol; Procaterol; ractopamine; reproterol; Ritodrine; Ro 363; salmeterol; salmeterol xinafoate; Terbutaline; tetramethylpyrazine; tizanidine or tizanidine hydrochloride; Tretoquinol; tulobuterol; Xamoterol; or zinterol. Additional non-limiting examples include Apraclonidine, Bitolterol Mesylate, Brimonidine or Brimonidine tartrate, Dipivefrin (which is converted to epinephrine in vivo), Epinephrine, Ergotamine, Guanabenz, guanfacine, Metaproterenol, Metaraminol, Methoxamine, Methyldopa, Midodrine (a prodrug which is metabolized to the major metabolite desglymidodrine formed by deglycination of midodrine), Oxymetazoline, Phenylephrine, Phenylpropanolamine, Pseudoephedrine, alphamethylnoradrenaline, mivazerol, natural ephedrine or D(−)ephedrine, any one or any mixture of two, three, or four of the optically active forms of ephedrine, CHF1035 or nolomirole hydrochloride (CAS RN 138531-51-8), AJ-9677 or TAK677 ([3-[(2R)-[[(2R)-(3-chlorophenyl)-2-hydroxyethyl]amino]propyl]-1H-indol-7-yloxy]acetic acid), MN-221 or KUR-1246 ((−)-bis(2-{[(2S)-2-({(2R)-2-hydroxy-2-[4-hydroxy-3-(2-hydroxyethyl)phenyl]ethyl}amino)-1,2,3,4-tetrahydronaphthalen-7-yl]oxy}-N,N-dimethylacetamide)monosulfate or bis(2-[[(2S)-2-([(2R)-2-hydroxy-2-[4-hydroxy-3-(2-hydroxyethyl)-phenyl]ethyl]amino)-1,2,3,4-tetrahydronaphthalen-7-yl]oxy]-N,N-dimethylacetamide)sulfate or CAS RN 194785-31-4), levosalbutamol (CAS RN 34391-04-3), lofexidine (CAS RN 31036-80-3) or TQ-1016 (from TheraQuest Biosciences, LLC).

In further embodiments, a reported adrenergic antagonist, such as idazoxan or fluparoxan, may be used as an agent in combination with an HDac inhibitory agent as described herein.

In further embodiments, an agent in combination with an HDac inhibitory agent may be a reported modulator of carbonic anhydrase. Non-limiting examples of such an agent include acetazolamide, benzenesulfonamide, benzolamide, brinzolamide, dichlorphenamide, dorzolamide or dorzolamide HCl, ethoxzolamide, flurbiprofen, mafenide, methazolamide, sezolamide, zonisamide, bendroflumethiazide, benzthiazide, chlorothiazide, cyclothiazide, dansylamide, diazoxide, ethinamate, furosemide, hydrochlorothiazide, hydroflumethiazide, mercuribenzoic acid, methyclothiazide, trichloromethazide, amlodipine, cyanamide, or a benzenesulfonamide. Additional non-limitinge examples of such an agent include (4s-Trans)-4-(Ethylamino)-5,6-Dihydro-6-Methyl-4h-Thieno(2,3-B)Thiopyran-2-Sulfonamide-7,7-Dioxide; (4s-Trans)-4-(Methylamino)-5,6-Dihydro-6-Methyl-4h-Thieno(2,3-B)Thiopyran-2-Sulfonamide-7,7-Dioxide; (R)-N-(3-Indol-1-YI-2-Methyl-Propyl)-4-Sulfamoyl-Benzamide; (S)-N-(3-Indol-1-YI-2-Methyl-Propyl)-4-Sulfamoyl-Benzamide; 1,2,4-Triazole; 1-Methyl-3-Oxo-1,3-Dihydro-Benzo[C]Isothiazole-5-Sulfonic Acid Amide; 2,6-Difluorobenzenesulfonamide; 3,5-Difluorobenzenesulfonamide; 3-Mercuri-4-Aminobenzenesulfonamide; 3-Nitro-4-(2-Oxo-Pyrrolidin-1-Yl)-Benzenesulfonamide; 4-(Aminosulfonyl)-N-[(2,3,4-Trifluorophenyl)Methyl]-Benzamide; 4-(Aminosulfonyl)-N-[(2,4,6-Trifluorophenyl)Methyl]-Benzamide; 4-(Aminosulfonyl)-N-[(2,4-Difluorophenyl)Methyl]-Benzamide; 4-(Aminosulfonyl)-N-[(2,5-Difluorophenyl)Methyl]-Benzamide; 4-(Aminosulfonyl)-N-[(3,4,5-Trifluorophenyl)Methyl]-Benzamide; 4-(Aminosulfonyl)-N-[(4-Fluorophenyl)Methyl]-Benzamide; 4-(Hydroxymercury)Benzoic Acid; 4-Flourobenzenesulfonamide; 4-Methylimidazole; 4-Sulfonamide-[1-(4-Aminobutane)]Benzamide; 4-Sulfonamide-[4-(Thiomethylaminobutane)]Benzamide; 5-Acetamido-1,3,4-Thiadiazole-2-Sulfonamide; 6-Oxo-8,9,10,11-Tetrahydro-7h-Cyclohepta[C][1]Benzopyran-3-O-Sulfamate; (4-sulfamoyl-phenyl)-thiocarbamic acid O-(2-thiophen-3-yl-ethyl) ester; (R)-4-ethylamino-3,4-dihydro-2-(2-methoylethyl)-2H-thieno[3,2-E]-1,2-thiazine-6-sulfonamide-1,1-dioxide; 3,4-dihydro-4-hydroxy-2-(2-(4-2H-thieno[3,2-E]-1,2-thiazine-6-sulfonamide-1,1-dioxide; 3,4-dihydro-4-hydroxy-2-(4-methoxyphenyl)-2H-thieno[3,2-E]-1,2-thiazine-6-sulfonamide-1,1-dioxide; N-[(4-methoxyphenyl)methyl]2,5-thiophenedesulfonamide; 2-(3-methoxyphenyl)-2H-thieno-[3,2-E]-1,2-thiazine-6-sulfinamide-1,1-dioxide; (R)-3,4-didhydro-2-(3-methoxyphenyl)-4-methylamino-2H-thieno[3,2-E]-1,2-thiazine-6-sulfonamide-1,1-dioxide; (S)-3,4-dihydro-2-(3-methoxyphenyl)-4-methylamino-2H-thieno[3,2-E]-1,2-thiazine-6-sulfonamide-1,1-dioxide; 3,4-dihydro-2-(3-methoxyphenyl)-2H-thieno-[3,2-E]-1,2-thiazine-6-sulfonamide-1,1-dioxide; [2h-Thieno[3,2-E]-1,2-Thiazine-6-Sulfonamide,2-(3-Hydroxyphenyl)-3-(4-Morpholinyl)-1,1-Dioxide]; [2h-Thieno[3,2-E]-1,2-Thiazine-6-Sulfonamide,2-(3-Methoxyphenyl)-3-(4-Morpholinyl)-, 1,1-Dioxide]; Aminodi(Ethyloxy)Ethylaminocarbonylbenzenesulfonamide; N-(2,3,4,5,6-Pentaflouro-Benzyl)-4-Sulfamoyl-Benzamide; N-(2,6-Diflouro-Benzyl)-4-Sulfamoyl-Benzamide; N-(2-FLOURO-BENZYL)-4-SULFAMOYL-BENZAMIDE; N-(2-Thienylmethyl)-2,5-Thiophenedisulfonamide; N-[2-(1H-INDOL-5-YL)-BUTYL]-4-SULFAMOYL-BENZAMIDE; N-Benzyl-4-Sulfamoyl-Benzamide; or Sulfamic Acid 2,3-O-(1-Methylethylidene)-4,5-O-Sulfonyl-Beta-Fructopyranose Ester.

In yet additional embodiments, an agent in combination with an HDac inhibitory agent may be a reported modulator of a catechol-O-methyltransferase (COMT), such as floproprion, or a COMT inhibitor, such as tolcapone (CAS RN 134308-13-7), nitecapone (CAS RN 116313-94-1), or entacapone(CAS RN 116314-67-1 or 130929-57-6).

In yet further embodiments, an agent in combination with an HDac inhibitory agent may be a reported modulator of hedgehog pathway or signaling activity such as cyclopamine, jervine, ezetimibe, regadenoson (CAS RN 313348-27-5, or CVT-3146), a compound described in U.S. Pat. No. 6,683,192 or identified as described in U.S. Pat. No. 7,060,450, or CUR-61414 or another compound described in U.S. Pat. No. 6,552,016.

In other embodiments, an agent in combination with an HDac inhibitory agent may be a reported modulator of IMPDH, such as mycophenolic acid or mycophenolate mofetil (CAS RN 128794-94-5).

In yet additional embodiments, an agent in combination with an HDac inhibitory agent may be a reported modulator of a sigma receptor, including sigma-1 and sigma-2. Non-limiting examples of such a modulator include an agonist of sigma-1 and/or sigma-2 receptor, such as (+)-pentazocine, SKF 10,047 (N-allylnormetazocine), or 1,3-di-o-tolylguanidine (DTG). Additional non-limiting examples include SPD-473 (from Shire Pharmaceuticals); a molecule with sigma modulatory activity as known in the field (see e.g., Bowen et al., Pharmaceutica Acta Helvetiae 74: 211-218 (2000)); a guanidine derivative such as those described in U.S. Pat. Nos. 5,489,709; 6,147,063; 5,298,657; 6,087,346; 5,574,070; 5,502,255; 4,709,094; 5,478,863; 5,385,946; 5,312,840; or 5,093,525; WO9014067; an antipsychotic with activity at one or more sigma receptors, such as haloperidol, rimcazole, perphenazine, fluphenazine, (−)-butaclamol, acetophenazine, trifluoperazine, molindone, pimozide, thioridazine, chlorpromazine and triflupromazine, BMY 14802, BMY 13980, remoxipride, tiospirone, cinuperone (HR 375), or WY47384.

Additional non-limiting examples include igmesine; BD1008 and related compounds disclosed in U.S. Publication No. 20030171347; cis-isomers of U50488 and related compounds described in de Costa et al, J. Med. Chem., 32(8): 1996-2002 (1989); U101958; SKF10,047; apomorphine; OPC-14523 and related compounds described in Oshiro et al., J Med Chem.; 43(2): 177-89 (2000); arylcyclohexamines such as PCP; (+)-morphinans such as dextrallorphan; phenylpiperidines such as (+)-3-PPP and OHBQs; neurosteroids such as progesterone and desoxycorticosterone; butryophenones; BD614; or PRX-00023. Yet additional non-limiting examples include a compound described in U.S. Pat. Nos. 6,908,914; 6,872,716; 5,169,855; 5,561,135; 5,395,841; 4,929,734; 5,061,728; 5,731,307; 5,086,054; 5,158,947; 5,116,995; 5,149,817; 5,109,002; 5,162,341; 4,956,368; 4,831,031; or 4,957,916; U.S. Publication Nos. 20050132429; 20050107432; 20050038011, 20030105079; 20030171355; 20030212094; or 20040019060; European Patent Nos. EP 503 411; EP 362 001-A1; or EP 461 986; International Publication Nos. WO 92/14464; WO 93/09094; WO 92/22554; WO 95/15948; WO 92/18127; 91/06297; WO01/02380; WO91/18868; or WO 93/00313; or in Russell et al., J Med Chem.; 35(11): 2025-33 (1992) or Chambers et al., J. Med Chem.; 35(11): 2033-9 (1992).

Further non-limiting examples include a sigma-1 agonist, such as IPAG (1-(4-iodophenyl)-3-(2-adamantyl)guanidine); pre-084; carbetapentane; 4-IBP; L-687,384 and related compounds described in Middlemiss et al., Br. J. Pharm., 102: 153 (1991); BD 737 and related compounds described in Bowen et al., J Pharmacol Exp Ther., 262(1): 32-40 (1992)); OPC-14523 or a related compound described in Oshiro et al., J Med Chem.; 43(2): 177-89 (2000); a sigma-1 selective agonist, such as igmesine; (+)-benzomorphans, such as (+)-pentazocine and (+)-ethylketocyclazocine; SA-4503 or a related compound described in U.S. Pat. No. 5,736,546 or by Matsuno et al., Eur J Pharmacol., 306(1-3): 271-9 (1996); SK&F 10047; or ifenprodil; a sigma-2 agonist, such as haloperidol, (+)-5,8-disubstituted morphan-7-ones, including CB 64D, CB 184, or a related compound described in Bowen et al., Eur. J. Parmacol. 278:257-260 (1995) or Bertha et al., J. Med. Chem. 38:4776-4785 (1995); or a sigma-2 selective agonist, such as 1-(4-fluorophenyl)-3-[4-[3-(4-fluorophenyl)-8-azabicyclo[3.2.1]oct-2-en-8-yl]-1-butyl]-1H-indole, Lu 28-179, Lu 29-253 or a related compound disclosed in U.S. Pat. Nos. 5,665,725 or 6,844,352, U.S. Publication No. 20050171135, International Patent Publication Nos. WO 92/22554 or WO 99/24436, Moltzen et al., J. Med Chem., 26; 38(11): 2009-17 (1995) or Perregaard et al., J Med Chem., 26; 38(11): 1998-2008 (1995).

Alternative non-limiting examples include a sigma-1 antagonist such as BD-1047 (N(−)[2-(3,4-dichlorophenyl)ethyl]-N-methyl-2-(dimethylamin-o)ethylamine), BD-1063 (1(−)[2-(3,4-dichlorophenyl)ethyl]-4-methylpiperazine, rimcazole, haloperidol, BD-1047, BD-1063, BMY 14802, DuP 734, NE-100, AC915, or R-(+)-3-PPP. Particular non-limiting examples include fluoxetine, fluvoxamine, citalopram, sertaline, clorgyline, imipramine, igmesine, opipramol, siramesine, SL 82.0715, imcazole, DuP 734, BMY 14802, SA 4503, OPC 14523, panamasine, or PRX-00023.

Other non-limiting examples of an agent in combination with an HDac inhibitory agent include acamprosate (CAS RN 77337-76-9); a growth factor, like LIF, EGF, FGF, bFGF or VEGF as non-limiting examples; octreotide (CAS RN 83150-76-9); an NMDA modulator like DTG, (+)-pentazocine, DFIEA, Lu 28-179 (1′-[4-[1-(4-fluorophenyl)-1H-indol-3-yl]-1-butyl]-spiro[isobenzofuran-1(3H), 4′piperidine]), BD 1008 (CAS RN 138356-08-8), ACEA1021 (Licostinel or CAS RN 153504-81-5), GVI50526A (Gavestinel or CAS RN 153436-22-7), sertraline, clorgyline, or memantine as non-limiting examples; or metformin.

Of course a further combination therapy may also be that of an HDac inhibitory agent with a non-chemical based therapy. Non-limiting examples include the use of psychotherapy for the treatment of many conditions described herein, such as the psychiatric conditions, as well as behavior modification therapy such as that use in connection with a weight loss program.

As described herein, a combination of a neurogenesis modulating agent and an HDac inhibitory agent may be used to modulate one or more aspects of neurogenesis, e.g., proliferation, differentiation, migration and/or survival, to a greater degree than either of the agents alone. For example, in some embodiments, a neurogenesis modulating HDac inhibitory agent that enhances differentiation, but not other aspects of neurogenesis is administered in combination with one or more compounds that enhance one or more additional aspects of neurogenesis, such as proliferation, differentiation, migration, inhibition of astrocytes, and/or survival. Advantageously, administering a combination of neurogenesis modulating agents having complementary modes of action enhances therapeutic efficacy and/or other aspects of treatment.

In some embodiments, a neurogenesis modulating agent is administered in combination with another agent that binds to, modifies, and/or stimulates an endogenous agent that enhances the potency (IC₅₀), affinity (K_{d}), and/or effectiveness of the neurogenesis modulating agent. Non-limiting examples include an additional agent administered in combination with an HDac inhibitory agent such that the effectiveness of either agent, including potency, affinity, or other property, is enhanced.

Methods for evaluating the neurogenesis modulating activity of neuromodulating HDac inhibitors in vitro, as well as the efficacy of neuromodulating HDac inhibitors in the treatment of various CNS disorders, including depression and/or anxiety, cognitive disorders, and cognitive function, are described in the references cited herein, as well as in the experimental examples provided below, which are non-limiting and merely representative of various aspects of the invention.

The methods disclosed herein may be utilized as one component in the providing of medical care to an animal subject or human patient. One non-limiting example is the application of a method disclosed herein in combination with one or more diagnostic methods (e.g. diagnostic services) as medical care. Thus the disclosure includes a method in the medical care of a subject or patient, the method comprising administration of an HDac inhibitory agent, alone or in combination as described herein. A method in the medical care of a subject or patient may thus include any method comprising administration of an HDac inhibitory agent as disclosed herein.

The medical care method optionally includes determination, such as by diagnosis or measurement as non-limiting examples, of the need for treatment with an HDac inhibitory agent, alone or in combination, as disclosed herein. In some embodiments, the determination is selection of an HDac inhibitory agent as suitable or preferable over another HDac inhibitory agent or another agent for the treatment of a disease or condition as described herein. Non-limiting examples include selection of an HDac inhibitory agent for the treatment of cancer or epilepsy in a subject or patient.

In addition to selection based on efficacy or value in treating a disease or condition, the selection of an HDac inhibitory agent may be based upon improved outcome in cognitive function, such as by lessening or reducing a decline or decrease of cognitive function in a subject or patient treated with the agent, as described herein. The selection may be in comparison to another agent, optionally another HDac inhibitory agent, or may be based upon recognition or realization of an advantageous phenotype or result in relation to cognitive function. In additional embodiments, the selection is based upon an HDac inhibitory agent as suitable or preferable for 1) maintenance or stabilization of cognitive function, 2) treating a mood disorder, 3) reducing or inhibiting aberrant differentiation, proliferation and/or migration of neural cells in a tissue, and/or 4) maintaining, stabilizing, stimulating, or increasing neurodifferentiation in a cell or tissue in a subject or patient, as described herein. Again, the selection may be in comparison to another agent or may be based upon recognition or realization of one or more advantageous phenotypes or results as listed above.

Therefore, a determination to administer or deliver an HDac inhibitory agent may be based upon one or more activities of the agent as disclosed herein. Non-limiting examples include determination to provide or deliver an HDac inhibitory agent, optionally to the exclusion of one or more other agents, to lessen or reduce a decline or decrease of cognitive function. Treatment with an HDac inhibitory agent may be in place of, or to the exclusion of, another agent or agents which do not result in a lessening or reducing of a decline or decrease in cognitive function. As a non-limiting example, a determination may be made to administer an HDac inhibitory agent, in place of another agent or agents, to a subject or patient in need of anti-cancer chemotherapy and/or radiation therapy. As an additional non-limiting example, a determination may be to administer an HDac inhibitory agent, in place of another agent or agents, to a subject or patient with epilepsy or with seizures associated with epilepsy.

In some cases, the determination to administer or provide an HDac inhibitory agent may be based upon recognition or reports of the HDac inhibitory agent as lessening or reducing a decline or decrease in cognitive function associated with epilepsy. Additional non-limiting examples include a determination based upon recognition or reports of one or more advantageous phenotypes or results as described above.

In other embodiments, a medical care method comprises determination of a patient as suitable for, or likely to benefit from, treatment with an HDac inhibitory agent recognized or reported as producing an improved outcome in cognitive function, such as by lessening or reducing a decline or decrease of cognitive function in a subject or patient treated with the agent, as described herein, in comparison to another agent. The determination may be by any means known to the skilled person, including knowledge of the course of a disease or condition (e.g. the pathology thereof) and/or assessment by a test or assay as described herein. Alternatively, the determination may be of a patient as suitable for, or likely to benefit from, treatment with an HDac inhibitory agent recognized or reported as producing a phenotype or result of 1) maintenance or stabilization of cognitive function, 2) treating a mood disorder, 3) reducing or inhibiting aberrant differentiation, proliferation and/or migration of neural cells in a tissue, and/or 4) maintaining, stabilizing, stimulating, or increasing neurodifferentiation in a cell or tissue in a subject or patient.

So a determination of a subject or patient as in need of, or a suitable recipient of, or likely to benefit from, administration of an HDac inhibitory agent may be based upon one or more phenotypes or results as disclosed herein. Non-limiting examples include determination of a need for treatment with an HDac inhibitory agent, optionally to the exclusion of one or more other agents, to lessen or reduce a decline or decrease of cognitive function, or to produce one or more of the described phenotypes or results.

A medical care method disclosed herein may include any diagnosis or measurement suitable for determining the choice or delivery of an HDac inhibitory agent, alone or in combination, to a subject or patient. In some embodiments, the determination is made by a medical doctor, nurse or other health care provider, or those working under his/her instruction. The determination may also have been made by personnel of a health insurance or health maintenance organization in approving the performance of the diagnosis or measurement as a basis to request reimbursement or payment for the performance. In some cases, the determination may be made in light of recognition or reports regarding the activities of an HDac inhibitory agent, such as those listed herein as non-limiting examples, as provided by a manufacturer or distributor of the agent. Non-limiting examples of a manufacturer or distributor include a pharmaceutical or chemical company.

Having now generally described the invention, the same will be more readily understood through reference to the following examples which are provided by way of illustration, and are not intended to be limiting of the disclosed invention, unless specified.

### EXAMPLES

### Example 1

### Effect of HDac Inhibitors on Neuronal Differentiation of Human Neural Stem Cells

Experiments were carried out essentially as described in U.S. Provisional Application No. 60/697,905 (incorporated by reference). Briefly, human neural stem cells (hNSCs) were isolated and grown in monolayer culture, and then plated and treated with varying concentrations of test compounds. Cells were stained with the neuronal antibody TUJ-1. Mitogen-free test media with a neuronal differentiating agent served as a positive control for neuronal differentiation, and basal media without growth factors served as a negative control.

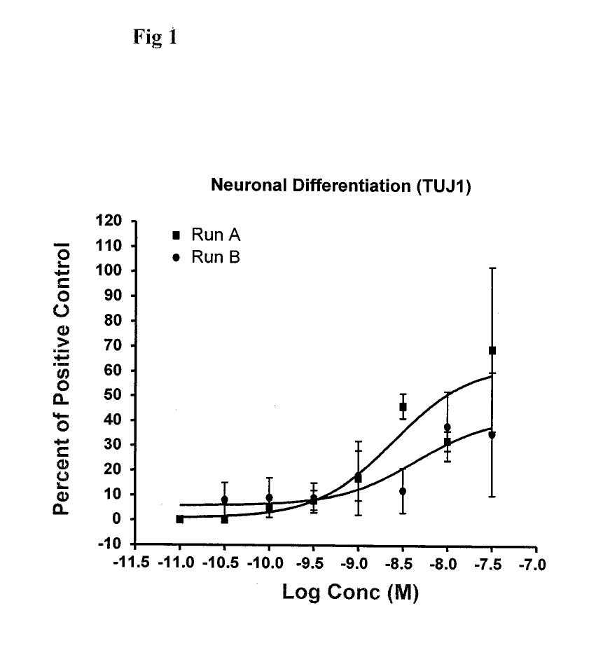

The results are shown in FIG. 1, which shows differentiation of human neural stem cells into neurons in the presence of trichostatin A. The data indicate that the HDac inhibitor trichostatin A permits differentiation of cells along a neuronal lineage.

### Example 2

### Effect of HDac Inhibitors on Astrocyte Differentiation of hNSCs

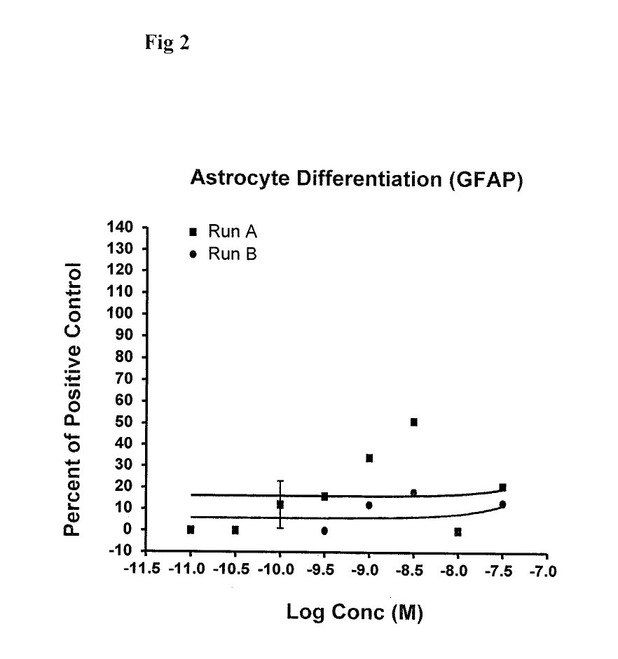

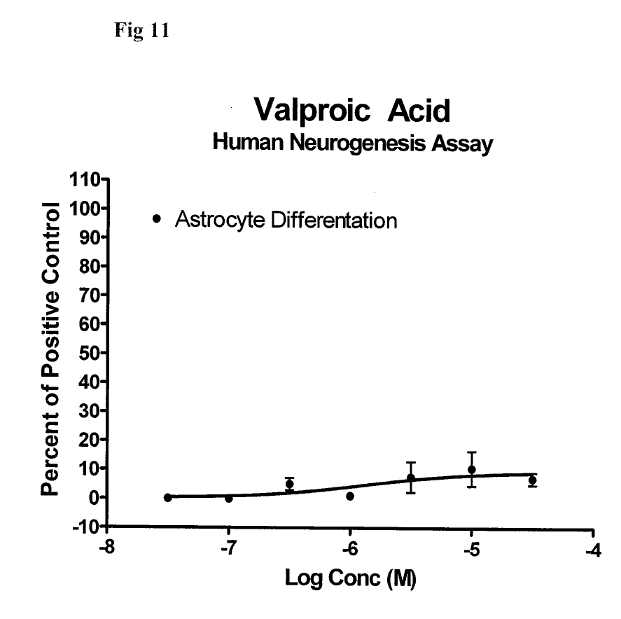

Experiments were carried out as described in Example 1, except the positive control for astrocyte differentiation contained mitogen-free test media with an astrocyte differentiating agent, and cells were stained with the astrocyte antibody GFAP. Results are shown in FIGS. 2 and 11, which show the lack of astrocyte differentiation of human neural stem cells in the presence of trichostatin A and valproic acid, respectively. The data indicate that HDac inhibitors inhibit the differentiation of human neural stem cells along an astrocytic lineage.

### Example 3

### Effect of HDac Inhibitors on Survival of Human Neural Stem Cells

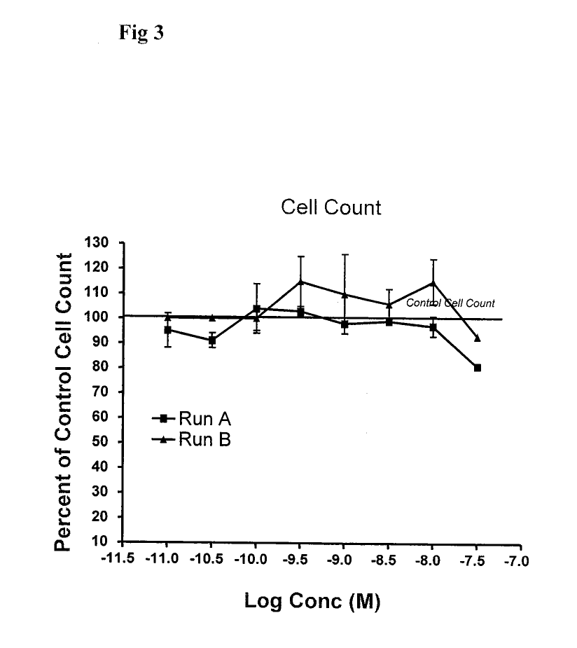

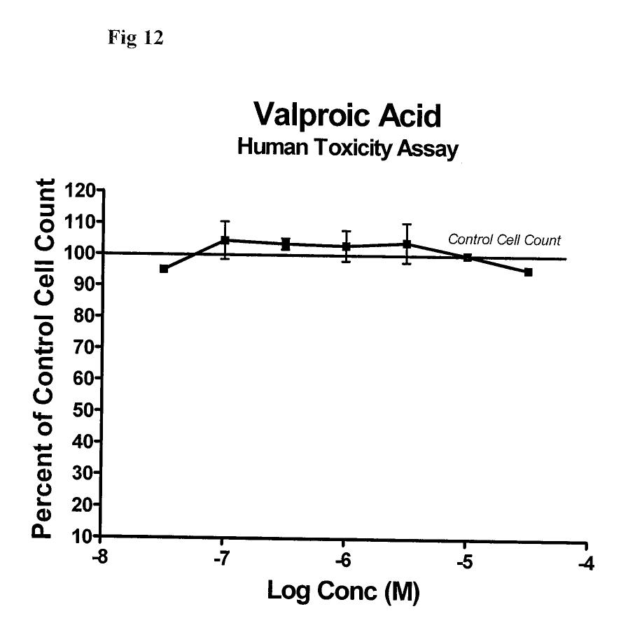

Experiments were carried out as described in Example 1, except that the cells were stained with a nuclear dye (Hoechst 33342). Results are shown in FIGS. 3 and 12, which show the maintenance of cell number human neural stem cells over a seven day period by trichostatin A and valproic acid, respectively. The data indicate that HDac inhibitors are non-toxic to human neural stem cells over an extended period of time.

### Example 4

### In Vitro Rodent Gene Reporter Assay

Experiments were carried out essentially as described in U.S. Provisional Application No. 60/697,905 (incorporated by reference). Briefly, cultured rodent neural stem cells (rNSC) were transfected by electroporation with a vectors containing promoters specific for the rat NeuroD1, GluR2, NFH and GAP43 genes linked to the fluorescent reporter protein DsRed. All gene reporter constructs were cloned in the same lentiviral vector backbone, and mixed with Nucleofactor solution for electroporation. A GFP vector control was used in parallel to visualize effectiveness of electroporation. Transfected rNSCs were then suspended in test media containing varying concentrations of the HDac inhibitors trichostatin A, valproic acid, MS-275 or apicidin, and a mixture of 0.5 μg renilla luciferase and 5 μg promoter-specific sea pansy luciferase. The cells were incubated in 5% CO₂ at 37° C. for 2 days, and then lysed, whereupon the cell extracts were read on a Tecan Genios Pro reader to detect the promoter-specific activation of the reporter constructs.

Valproic acid, MS-275 and apicidin were neurogenic, as indicated by activation of the rat NeuroD1, GluR2, NFH and GAP43 promoters. The results are shown in FIGS. 4-9. These data show that the HDac inhibitors MS-275 and valproic acid promote expression of promoters for the neurofilament high (NFH) and growth associated protein 43 (GAP43) genes, which are activated during neuronal differentiation. These data further indicate that HDac inhibitors permit, and may promote, differentiation of human neural stems cells along a neuronal lineage.

### Example 5

### In Vivo Chronic Dosing Studies

Male Fischer F344 rats were treated with 300 mg/kg of valproic acid for 28 days, and then anesthetized and killed by transcardial perfusion of 4% paraformaldehyde at day 28. Brains were rapidly removed and stored in 4% paraformaldehyde for 24 hours and then equilibrated in phosphate buffered 30% sucrose. Free floating 40 micron sections were collected on a freezing microtome and stored in cyroprotectant. Antibodies against BrdU and cells types of interest (e.g., neurons, astrocytes, oligodendrocytes, endothelial cells) were used for detection of cell survival and differentiation. In brief, tissues were washed (0.01 M PBS), endogenous peroxidase blocked with 1% hydrogen peroxide, and incubated in PBS (0.01M, pH 7.4, 10% normal goat serum, 0.5% Triton X-100) for 2 hours at room temperature. Tissues were then incubated with primary antibody at 4° C. overnight. The tissues were then rinsed in PBS followed by incubation with biotinylated secondary antibody (1 hour at room temperature). Tissues were further washed with PBS and incubated in avidin-biotin complex kit solution at room temperature for 1 hour. Bound antibodies were visualized with fluorophores linked to streptavidin.

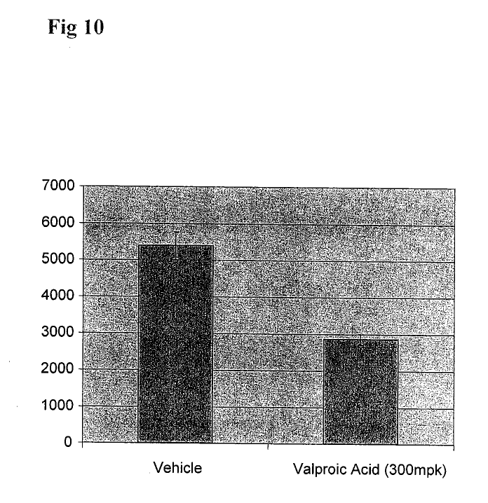

Cell counting and unbiased stereology was limited to the hippocampal granule cell layer proper and a 50 um border along the hilar margin that includes the neurogenic subgranular zone. The proportion of BrdU cells displaying a lineage-specific phenotype was determined by scoring the co-localization of cell phenotype markers with BrdU using confocal microscopy. Split panel and z-axis analysis was used for all counting. All counts were performed using multi-channel configuration with a 40× objective and electronic zoom of 2. When possible, 100 or more BrdU-positive cells were scored for each maker per animal. Each cell was manually examined in first full “z”-dimension and only those cells for which the nucleus was unambiguously associated with the lineage-specific marker were scored as positive. The total number of BrdU-labeled cells per hippocampal granule cell layer and subgranular zone were determined using diaminobenzadine stained tissues. Overestimation was corrected using the Abercrombie method for nuclei with empirically determined average diameter of 13 um within a 40 um section. As shown in FIG. 10, valproic acid substantially decreased proliferation of neural stem and/or progenitor cells.

These data indicate that the HDac inhibitor valproic acid inhibits proliferation of neural stem cells in the adult mammalian brain. However, valproic acid continues to promote neuronal differentiation as shown in FIG. 1 but does not promote astrocyte differentiation as shown in FIG. 11. Therefore, these data indicate that HDac inhibitors preferentially increase neurons while limiting or decreasing astrocytes.

### Example 6

### Characterization of In Vivo Neurogenesis: Assay Models for Diseases of the Central and Peripheral Nervous System

Depression Mood Disorders, and Other Conditions

The following in vivo assays are models of various diseases as described above. The assays may thus be used to assess an agent or a combination of agents as disclosed herein for treatment of a disease. Non-limiting examples of a disease include depression, mood disorder, or other condition disclosed herein.

Locomotor Activity

Open field activity during the light phase of the diurnal cycle is quantified via photoelectric cell monitoring in a Plexiglas cube open-field arena (45 cm×45 cm×50 cm high with infra-red (I/R) array, Hamilton-Kinder San Diego, Calif.). Measurements are collected for 30 minutes (6 blocks of 5 min): ambulatory distance in center and periphery; ambulatory time in center and periphery; total time in center and periphery; rearing in center and periphery; the number of zone entries; and total distance. Testing begins 30 minutes after injection with an HDac inhibitor, such as trichostatin A, valproic acid, MS-275 or apicidin.

Forced Swim Test

Active motor behavior is measured in a swim tank, this test being a modification of that described by Porsolt, R. D., Bertin, A., Jalfree, M. Arch. Int. Pharmacodyn Ther. 229 (1977) 327-336. The animal is placed into the swim tank (38 cm deep). The swim test consists of two phases; a 15 minute pretest and a 5 minute test 24-hours later. Activity is quantified by measuring three aspects of behavior: (1) immobility, defined as an absence of movement other than what is required to remain afloat, (2) swimming, defined as horizontal movement greater than what is required to remain afloat and (3) climbing, vertical movement greater than what is required to remain afloat. The predominant behavior is scored every 5 seconds by trained observers for a total of 5 minutes.

Tail Suspension

Lack of active motor behavior is measured in a mouse that is suspended by its tail to a metal bar. The test to be used is the same as that described by Steru L, Chermat R, Thierry B, Simon P, Psychopharmacology, 1985, 85(3):367-70. The animal is suspended from its tail on a metal bar located 30 cm above a flat surface for 5-10 minutes. Adhesive tape is used to suspend the mouse from its tail on the metal bar. Immobility is quantified by measuring the amount of time when no whole body movement is observed. The animals are very alert during the test, and their breathing appears normal. They respond to any form of sensory stimulus (especially sound or smell) even when they are immobile. As soon as the animal is detached from the bar, the animal resumes its usual behaviors/activity. Antidepressants reverse the immobility in the test.

Chronic Unpredictable Stress

In this paradigm, rats are exposed to a series of mild stressors, such as food and/or water restriction for 12 hours, 1 hour of restraint stress (see Protocol RS-R), tilted or soiled cage for 12 hours, reverse light-dark cycle for 12 hours, or group housing overnight if the rats have been housed singly. The rats are subjected to only one or two stressors within a 24 hour period and the entire protocol lasts from 5 to 15 days. The stressors are presented in a random order and unpredictably to the subjects. The unpredictability of the occurrence of the mild stressors is the key to the stressful experience considering that all of the stressors are very mild.

Learned Helplessness

On Day 1, twenty-four hours before exposure to inescapable shock, all animals are exposed to a 2 hour strobe stress session consisting of twelve, 1 minute exposures to a strobe light every 10 minutes. Animals sit in the dark in between strobe light exposures and are returned to home cages 20 minutes after last strobe exposure. On day 2, rats are subjected to 60 inescapable electric foot shocks (0.8 mA; 15 sec duration; average interval 45 sec). On day 3, a two-way conditioned avoidance test (i.e. learned helplessness test) is performed as to determine whether the rats will show the predicted escape deficits to foot shock. Learned helplessness behavioral tests are performed with an automated system (Hamilton-Kinder, San Diego, Calif.). This apparatus is divided into two compartments by a retractable door. This test session consists of 30 trials in which the animals are exposed to 30 electric foot shocks (0.8 mA; 3 sec duration, average intervals ranging from 22 to 38 sec) preceded by a 3 sec conditioned stimulus tone that remains on until the shock is terminated. The rats can switch chamber and escape the shock at any time during the trials. Rats with >20 escape failures in the 30 trials are regarded as having reached the criterion and are used for further experiments. It is estimated that approximately 75% of the rats reach this criterion. Imipramine (10 mg/kg, i.p., twice per day), saline and test compounds are administered 1 day after the conditioned avoidance screening test. The compounds are administered for 7 days until 1 day before the active avoidance behavioral tests are performed again.

The second two-way active avoidance testing (30 trials, 0.8 mA shock, 30 s shock proceeded by 3 s CS tone) is then conducted and the numbers of escape failures and escape latency in each 30 trials are recorded by the computer system. An escape failure is defined as the failure of the rat to cross to the non-electrified chamber within the 33 second interval (3 sec tone with door open followed by 30 sec of shock). Rarely, animals cross before the shock, and this is defined as avoidance. Animals with greater than 20 failures in both the post-test and the final test are considered helpless. Escape latencies are calculated from the beginning of the tone since animals have the opportunity to escape starting at that time point. Consequently, an escape failure for any given trial gives a latency of 33 seconds. An animal crossing at 32 seconds in a given trial is scored as 32 second latency without an escape failure.

Olfactory Bulbectomy

Male Sprague Dawley rats of approx. 10 weeks are placed in the Open Field for 15 min and animals are matched re activity over all groups (N=108 rats). Two weeks after arrival all rats receive either Olfactory Bulbectomy (N=60) or Sham (N=48) surgery. Two weeks later the experiment starts with an Open Field test of 15 min immediately followed by the first daily injection (IP) of the consecutive daily treatment for 14 days. The last treatment (DAY 14-28) is followed (30 min) by an Open Field test of 15 min. Body weight of all animals is measured weekly.

Novelty Suppressed Feeding Assay

Twenty-four hours prior to behavioral testing, all food is removed from the home cage. At the time of testing a single pellet is placed in the center of a novel arena. Animals are placed in the corner of the arena and latency to eat the pellet is recorded. Compounds are generally administered 30 minutes prior to testing. Animals receive compound daily for 21 days and testing is performed on day 21.

### Example 7

### Characterization of In Vivo Neurogenesis: Cognition (Cognitive Function) Assays

The following assay models may be used to assess cognitive function or other conditions as described herein. The applicability of these assays to other diseases and conditions are known to the skilled person.

Active Avoidance

The apparatus consists of a shuttle-box divided into a lighted (white) compartment and a dark (black) compartment. Each compartment is 24 cm×16 cm×19 cm and is equipped with a grid floor composed of 14 bars, 0.5 cm in diameter, spaced 2 cm apart. The compartments are connected by an opening with a sliding door. The compartment designated as the shock compartment is fitted with a light and/or tone generator to produce the conditioned stimulus (CS). Apparatus control and response recording are computer automated (Hamilton-Kinder, San Diego, Calif.).

On the first trial the CS (either tone or light) is presented simultaneously with a scrambled AC shock (0.3-1.0 mA [12-240 V]) while animals are in the dark compartment of the apparatus. As soon as the animal enters the safe compartment, the door is closed for a 30 second intertrial interval prior to placement in the start/shock compartment for the next trial. For subsequent trials, animals are placed in the dark start chamber, exposed to the CS, and given 10 sec to shuttle into the white safe compartment. If the animal fails to respond to the CS within 10 seconds, a shock is turned on and is terminated when the animal escapes into the safe compartment or after 30 sec have elapsed. If an animal does not avoid (enter the safe compartment during CS only exposure) or escape (enter the safe compartment during the CS+shock) the shock, it is gently pushed through the door into the safe compartment. Rats are given 4-8 trials/day with memory retention tests occurring on days 1-7 following the acquisition day. The number and latency of avoidance and escape responses are recorded.

Passive Avoidance

The apparatus is the same as used in active avoidance testing. For the training trial, the animals are placed in the white compartment facing the door (after 90 seconds of adaptation to the compartment). The door is opened and the time taken to enter the shock compartment is recorded. When the animal steps into the dark section with all four paws, the door is closed, and a 0.5-1.5 sec 0.3-1.0 mA (72-240 V), footshock is given. After 5 sec, the rat is removed and placed in its home cage. At the time of the testing trial (usually 1-7 days later), the animal is returned to the white compartment. Latency to enter the dark compartment is recorded, but the animal is not shocked.

Object Recognition

The apparatus consists of an open field (45×45×50 cm high) made of polycarbonate. Triplicate copies are used of the objects to be discriminated. Care is taken to ensure that the pair of objects to be tested are made from the same material so that they can not be distinguished readily by olfactory cues although they have very different appearances. Each test session consists of two phases. In the initial familiarization phase, two identical objects (A1 and A2) are placed in the far corners of the box arena. A rat is then placed in the middle of the arena and allowed 15 minutes to explore both objects. Exploration of an object is defined as directing the nose to the object at a distance of less than 2 cm and/or touching it with the nose. After a delay of 48-hours, the rat is re-introduced to the arena (“test phase”). The box now contains a third identical copy of the familiar object (A3) and a new object (B). These are placed in the same locations as the sample stimuli, whereby the position (left or right) of the novel object in the test phase is balanced between rats. For half the rats, object A is the sample and object B is the novel alternative. The test phase is 15 minutes in duration, with the first 30 seconds of object interaction used to determine preference scores. Any animal with less than 15 seconds of object exploration are excluded from analysis.

Object Location

In this test two copies of the same object (A) were used. In the sample phase the rat was exposed to objects A1 and A2 which were placed in the far corners of the arena (as in the object recognition test). The animal was allowed to explore both objects during a sample phase of 3 min, and the amount of exploration of each object recorded by the experimenter. After a delay of 5 min the test phase began. In the test phase object A3 was placed in the same position as A1 had occupied in the sample phase and object A4 was placed in the corner adjacent to the original position of A2, so that the two objects A3 and A4 were in diagonal corners. Thus, both objects in the test phase were equally familiar but only one had changed location. There was only one session of testing and during the choice phase the position of the moved object was counterbalanced between rats.

Visual Discrimination in Y-Maze

The apparatus used is a Y-maze, with each 3 equally long arms (61×14 cm) covered by smoked Plexiglas. At the end of one arm is a start box ( 11×14 cm) separated from the stem arm by a manually activated guillotine door. Rats are food-restricted according to protocol FR-R. Rats are given daily sessions of 3 to 5 trials. Day 1 of training consists of adaptation to the maze where the rats are allowed to explore the maze for 5 min, and food pellets are available in each arm. On day 2, each rat is placed in the start box with the door closed. The door is opened after 5 sec, and the rat is allowed to choose either the right or left arm of the maze to obtain a food pellet reward with pellets available in both arms. On day 3, each rat receives 6 trials in sets of 3 rats. Now, one arm is closed at the choice point, no discriminative stimulus is present, and two food pellets are available in the open goal box. The rat will be placed in the start box for only 15 sec, and which arm is open is determined by a counterbalanced sequence of left and right. On day 4, the same procedure will be used as on day 3 except that now a light will be illuminated, both arms are open but only the arm open on day 3 is baited, and 10 trials will be conducted. Rats will be tested in the maze for 10 trials, 6 days in succession. Rats will be tested in sets of 3 rats (i.e., the investigator will test the first rat in each set on its first trial, then the second rat on its first trial, etc., until 10 trials are completed for each of the 3 rats with an intertrial interval of approximately 1.5 min). For each trial, the latency to find the food pellets and the number of correct arm choices are recorded. Animals are given their daily food ration after each day's behavioral testing session is completed.

Trace Cued Contextual Fear Conditioning

Training: Subjects are placed into a conditioning chamber and allowed 2 minutes to explore the chamber (45 cm×45 cm×50 cm, Hamilton Kinder, San Diego Calif.). The CS tome is then presented for 30 seconds. During the last second of the CS tone the US footshock is administered. The first CS and US pairing were separated by a delay of 2.5 seconds. Animals received a total of four sequential CS and US pairings. Animals are then allowed to explore the cage for 2 additional minutes. All animals are then removed and returned to their home cage. Animals are scored for the presence or absence of freezing (absence of any movement except breathing).

Testing: Auditory cued fear test sessions occur on day 2. Subjects are allowed to explore the novel environment for 3 minutes without CS presentation, followed by 3 minutes of continuous CS presentation. Following the termination of the CS, the subjects were allowed to explore the novel context for an additional 90 seconds to determine if the subjects learned that the tone signaled an upcoming shock in the trace procedure. Freezing is scored at 10-sec intervals throughout the entire session (7.5 min). On day 3, subjects were returned to the original training room for the contextual fear test. Each subject was placed in the chamber were training took place and freezing behavior was recorded at 10-sec intervals over the 5-min test session. All conditions in the room were identical to the training day with the exception that the CS and US were not presented.

### Example 8

### Characterization of In Vivo Neurogenesis: Anxiety Assays

The following assay models may be used in relation to anxiety or other conditions as described herein. The applicability of these assays to other diseases and conditions are known to the skilled person.

Defensive Burying

In this paradigm, rats are first extensively handled to habituate them to handling and to the experimenter. Then, rats are placed in the testing cage for 45 minutes on two consecutive days to habituate them to the environment before the actual test occurs. The probe is not presented during habituation. During the test, the rat is exposed to a metal rod (i.e., probe) that delivers shock (1.5 mA) when touched by the subject. As soon as the shock is delivered (duration of shock less than 1 sec), the subject withdraws away from the probe and the experimenter switches off the current so further contacts with the rod do not lead to any further shocks. After this aversive experience, the rat starts pushing bedding material towards or over the rod. The animal's behavior is monitored by the experimenters for 15 minutes. After this 15 minute observation period, the subject is placed back in its home cage.

Emotional Stress Procedure

During the stress procedure, rats are tested in pairs. One rat is placed on each side of a two-compartmented Plexiglas chamber with a metal grid floor. The wall between the compartments is made of Plexiglas and has small holes (d=0.5 cm) so that the rats can see, hear and smell each other. In one of the compartments the rat is subjected to electrical foot-shocks and at the same time the other rat of the pair is subjected to the emotional stress of witnessing the reaction of the other rat to the foot shock procedure. Fifteen 1 second footshocks of 0.5 mA (40V), 50 Hz are administered within a 15 minute trial. The shock is delivered in a variable interval 60 second schedule, so that the shocked rat receives a shock once every 60 seconds on average for 15 minutes. Control rats are placed into the two compartments for 15 minutes without being exposed to the stress procedures.

Open Field Locomotion

Open field activity during both dark and light phases of the diurnal cycle is quantified via photoelectric cell monitoring in a Plexiglas cube open-field arena (45 cm×45 cm×50 cm high with infra-red (I/R) array, Hamilton-Kinder San Diego, Calif.). Measurements are collected for 90 mins (18 blocks of 5 min): ambulatory distance in center and periphery; ambulatory time in center and periphery; total time in center and periphery; rearing in center and periphery; the number of zone entries; and total distance.

Plus Maze

The plus maze apparatus has four arms (10×50 cm) at right angles to each other and is elevated 50 cm from the floor. Two of the arms have 40 cm high walls (enclosed arms) and two arms have no walls (open arms). The plus-maze is located in a quiet room that is dimmed to provide 22 to 350 lux of illumination for the open arms, and <1 lux within the enclosed arms. Rats are placed individually onto the center of the maze and allowed free access to all four arms for 5 min. Time spent on each arm is recorded automatically by photocell beams and a computer program. The data are presented as percentages of time spent in the open arms [open/(open+enclosed)] and the percentage of open arm entries [open/(open+enclosed)]. The maze is wiped clean with a damp cloth between each trial. Rats are observed through a window in the door as well as via an on-line display of the rats location on the computer monitor. Rats do not typically fall or jump off of the maze due to a small 0.5 cm border that discourages jumping. Rats in the wild typically jump these heights without injury.

Restraint Stress

The rat is placed in a clear, vented Plexiglas tube fitted with a tail slot to prevent unnatural body postures. The restraint period could vary from 20 minutes to 2 hours, and most frequently it is 20 minutes. The tube, designed to restrict nearly all movement, is placed on an absorbent pad to alleviate moisture buildup. Animals are monitored throughout the procedure to ensure that no physical harm results from movement. In the event that respiratory distress (highly unlikely since the tubes have several holes/gaps for ventilation), sustained struggling effort or unnatural body postures occur, the subject is immediately removed from the restrainer and placed in it's home cage.

Shock Stress

The rat is exposed to a mild footshock of 0.2-1.0 mA (12-120 V), 60 Hz 0.5 sec train duration. The shock will be delivered on a variable interval 40 sec schedule (i.e., the animal receives a shock once every 40 sec on average) for 10-15 min (corresponding to a total of 20 shocks). This is a shock level that rats will choose to receive in a conflict situation (Koob, G F, Braestrup, C., and Thatcher-Britton, K. The effects of FG 7142 and RO 15-1788 on the release of punished responding produced by chlordiazepoxide and ethanol in the rat. Psychopharmacology, 90:173-178, 1986) and that produces optimal avoidance performance in some learning situations such as two-way active avoidance. This is also the level of shock shown to reinstate drug self-administration following extinction paradigms.

Social Stress

Pairs of male (400 g) and female rats (250-300 g), termed “residents”, are housed in large boxes (48 cm×69 cm×51 cm) with sawdust-covered, stainless floors and unrestricted access to laboratory chow and water. Under these conditions the rats reliably establish appropriate reproductive and aggressive behavior after one month. After producing one litter, the females will have the uterine horns tied off to prevent future pregnancies. Aggressive behavior is readily observed when the resident male (female is temporarily removed) is confronted with a male intruder. The stressor here will be the exposure of a naive rat (intruder) to the resident male for 15-60 min. The resident typically attacks the intruder and within 90 sec the intruder assumes a submission posture and the intruder is removed by the experimenter to a screen enclosed container where the intruder is protected from any physical harm. Here the intruder can see and smell the resident and the resident will continue to threaten the intruder. No physical harm results from this procedure if the animals are removed in 90 sec. Any evidence of physical harm will cause the termination of the experiment, and the animal will be treated immediately with topical antibiotic and more serious injuries will be referred to veterinary staff for treatment. This exposure is sufficient to produce major increases in plasma stress hormone levels.

### Example 9

### In Vivo Acute Dosing Studies—Effect on Neurogenesis

Male Fischer F344 rats are injected with varying concentrations of HDac inhibitors, including trichostatin A, valproic acid, MS-275 and apicidin, within the range of about 10 nM to about 30 FM, +vehicle, or vehicle only (negative control), once daily for five days, followed by a single intraperitoneal injection with 100 mg/kg BrdU. Rats are then anesthetized and killed by transcardial perfusion of 4% paraformaldehyde at day 28. Brains are rapidly removed and stored in 4% paraformaldehyde for 24 hours and then equilibrated in phosphate buffered 30% sucrose. Free floating 40 micron sections are collected on a freezing microtome and stored in cyroprotectant. Antibodies against BrdU and cells types of interest (e.g., neurons, astrocytes, oligodendrocytes, endothelial cells) will also be used for detection of cell differentiation. In brief, tissues are washed (0.01 M PBS), endogenous peroxidase blocked with 1% hydrogen peroxide, and incubated in PBS (0.01M, pH 7.4, 10% normal goat serum, 0.5% Triton X-100) for 2 hours at room temperature. Tissues are then incubated with primary antibody at 4° C. overnight. The tissues are then rinsed in PBS followed by incubation with biotinylated secondary antibody (1 hour at room temperature). Tissues are further washed with PBS and incubated in avidin-biotin complex kit solution at room temperature for 1 hour. Various fluorophores linked to streptavidin are used for visualization. Tissues are washed with PBS, briefly rinsed in dH₂O, serially dehydrated and coverslipped.

Cell counting and unbiased stereology may include any brain region, but is generally limited to the hippocampal granule cell layer proper and a 50 um border along the hilar margin that includes the neurogenic subgranular zone. The proportion of BrdU cells displaying a lineage-specific phenotype is determined by scoring the co-localization of cell phenotype markers with BrdU using confocal microscopy. Split panel and z-axis analysis are used for all counting. All counts are performed using multi-channel configuration with a 40× objective and electronic zoom of 2. When possible, 100 or more BrdU-positive cells are scored for each maker per animal. Each cell is manually examined in first full “z”-dimension and only those cells for which the nucleus is unambiguously associated with the lineage-specific marker are scored as positive. Overestimation is corrected using the Abercrombie method for nuclei with empirically determined average diameter of 13 um within a 40 μm section. The method detects the ability of alacepril, azasetron, clorprenaline, flopropione, itopride HCl, meticrane, mosapride citrate, and rebamipide, and other neurogenesis modulators, to produce neurogenic effects with a rapid onset of action.

### Example 10

### Anti-Proliferative Effects on Human Neural Stem Cells

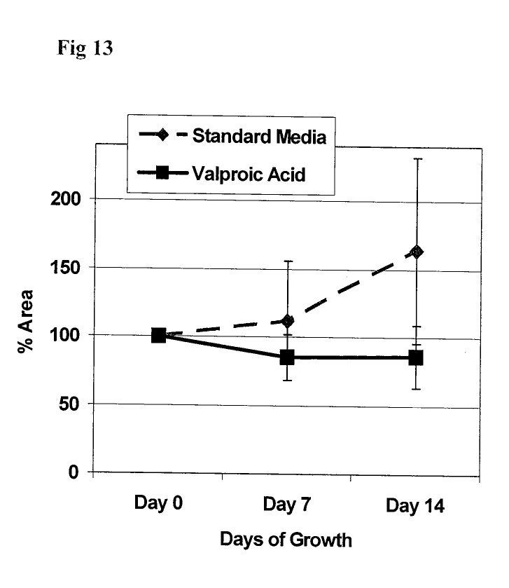

Experiments were carried out essentially as described in U.S. application Ser. No. 11/482,528, filed Jul. 7, 2006 (incorporated by reference). Briefly, human neural stem cells (hNSCs) were isolated and grown as clusters, then plated and treated with varying concentrations of test compounds. Cell clusters were imaged and their area measured over fourteen days. Basal media without growth factors served as a control for growth. Results are shown in FIG. 13, which shows an inhibition of normal growth by exposure of cell clusters to valproic acid. As the results described in Example 3 indicate, reduction in growth is not due to increased toxicity or reduced survival, and therefore the observed reduced growth of the cell clusters results from decreased proliferation of human neural stem cells in the presence of the HDac inhibitor. These data indicate that the HDac inhibitor inhibits human neural stem cell proliferation without increasing cell death.

### Example 11

### Embodiments

Some specific embodiments of the present invention are as follows:

In one embodiment, an adult human is treated with valproic acid, or a pharmaceutically acceptable salt or derivative thereof, at a daily dosage of about 20-60 mg/kg in order to treat depression and/or enhance cognitive function. In some embodiments, the patient suffers from a neurodegenerative condition. Pharmaceutically acceptable salts of valproic acid include, for example, valproate sodium (the sodium salt of valproic acid) and divalproex sodium (mixture of valproic acid and sodium valproate).

In another embodiment, an adult human is treated with valproic acid, or a pharmaceutically acceptable salt or derivative thereof, at a daily dosage of less than 20 mg/kg, 10 mg/kg, 5 mg/kg, or 1 mg/kg in order to treat depression and/or enhance cognitive function.

In another embodiment, an adult human is treated with MS-275, or a pharmaceutically acceptable salt or derivative thereof, at a daily dosage of about 0.1-1.0 mg/kg in order to treat depression and/or enhance cognitive function. In some embodiments, the patient suffers from a neurodegenerative condition.

In another embodiment, an adult human is treated with MS-275, or a pharmaceutically acceptable salt or derivative thereof, at a dosage of less than 0.1 mg/kg, 0.05 mg/kg, or 0.01 mg/kg at a frequency of less than once daily, 3 times weekly, once per week, or biweekly, in order to treat depression and/or enhance cognitive function.

In another embodiment, an adult human is treated with apicidin, or a pharmaceutically acceptable salt or derivative thereof, at a daily dosage of less than about 10 ug/kg, 5 ug/kg, or 1 ug/kg in order to treat depression and/or enhance cognitive function. In some embodiments, the patient suffers from a neurodegenerative condition.

In another embodiment, an adult human is treated with FK228, or a pharmaceutically acceptable salt or derivative thereof, at a daily dosage of less than about 0.35 mg/kg, 0.20 mg/kg, 0.1 mg/kg, 0.05 mg/kg, or 0.01 mg/kg in order to treat depression and/or enhance cognitive function. In some embodiments, the patient suffers from a neurodegenerative condition.

In another embodiment, an adult human is treated with FK228, or a pharmaceutically acceptable salt or derivative thereof, at a daily dosage of less than about 0.35 mg/kg, 0.20 mg/kg, 0.1 mg/kg, 0.05 mg/kg, or 0.01 mg/kg in order to treat depression and/or enhance cognitive function. In some embodiments, the patient suffers from a neurodegenerative condition.

In another embodiment, an adult human is treated with SAHA, or a pharmaceutically acceptable salt or derivative thereof, at a daily dosage of less than about 20 mg/kg, 10 mg/kg, 5 mg/kg, 1 mg/kg, 0.05 mg/kg, or 0.01 mg/kg in order to treat depression and/or enhance cognitive function. In some embodiments, the patient suffers from a neurodegenerative condition.

In another embodiment, an adult human is treated with trichostatin A, or a pharmaceutically acceptable salt or derivative thereof, at a daily dosage of less than about 20 mg/kg, 10 mg/kg, 5 mg/kg, 1 mg/kg, 0.05 mg/kg, or 0.01 mg/kg in order to treat depression and/or enhance cognitive function. In some embodiments, the patient suffers from a neurodegenerative condition.

All references cited herein, including patents, patent applications, and publications, are hereby incorporated by reference in their entireties, whether previously specifically incorporated or not.

Having now fully provided the instant disclosure, it will be appreciated by those skilled in the art that the same can be performed within a wide range of equivalent parameters, concentrations, and conditions without departing from the spirit and scope of the disclosure and without undue experimentation.

While the disclosure has been described in connection with specific embodiments thereof, it will be understood that it is capable of further modifications. This application is intended to cover any variations, uses, or adaptations of the disclosure following, in general, the disclosed principles and including such departures from the disclosure as come within known or customary practice within the art to which the disclosure pertains and as may be applied to the essential features hereinbefore set forth.

What is claimed is:

1. A method of lessening or reducing a decline or decrease of cognitive function in a subject or patient treated with anti-cancer chemotherapy and/or radiation therapy, said method comprising
administering an HDac inhibitory agent to said subject or patient to lessen or reduce a decline or decrease of cognitive function due to anti-cancer chemotherapy and/or radiation therapy.

2. The method of claim 1 wherein said lessening or reducing results in maintenance or stabilization of cognitive function in said subject or patient.

3. The method of claim 1 wherein said HDac inhibitory agent is administered before, or concurrent with, said anti-cancer chemotherapy and/or radiation therapy.

4. The method of claim 1 wherein said HDac inhibitory agent is trichostatin A, apicidin, MS-275, FK228, or SAHA.

5. The method of claim 1 wherein said chemotherapy comprises administration of a kinase inhibitor or a therapy independent of HDac inhibition.

6. The method of claim 1 wherein said patient is a human being diagnosed as having cancer.

7. A method of lessening or reducing a decline or decrease of cognitive function associated with epilepsy in a subject or patient, said method comprising
i) diagnosing said subject or patient as in need of lessening or reducing a decline or decrease in cognitive function associated with epilepsy, and
administering an HDac inhibitory agent to said subject or patient to lessen or reduce a decline or decrease of cognitive function in said subject or patient; or
ii) administering an HDac inhibitory agent, other than valproic acid, to said subject or patient to lessen or reduce a decline or decrease of cognitive function in said subject or patient.

8. The method of claim 7 wherein said lessening or reducing results in maintenance or stabilization of cognitive function in said subject or patient.

9. The method of claim 7 wherein said HDac inhibitory agent is trichostatin A, apicidin, MS-275, FK228, or SAHA.

10. The method of claim 7 wherein said subject or patient is a human being diagnosed as having epilepsy or having seizures associated with epilepsy.

11. A method of treating a nervous system disorder related to cellular degeneration, a psychiatric condition, cellular trauma and/or injury, or another neurologically related condition in a subject or patient, said method comprising
administering an HDac inhibitory agent, optionally in combination with another an HDac inhibitory agent and/or another neurogenic agent, to said subject or patient to produce an improvement in said disorder,
wherein said disorder is not epilepsy.

12. The method of claim 11, wherein said nervous system disorder related to cellular degeneration is selected from a neurodegenerative disorder, a neural stem cell disorder, a neural progenitor cell disorder, a degenerative disease of the retina, an ischemic disorder, and combinations thereof; or
wherein said nervous system disorder related to a psychiatric condition is selected from a neuropsychiatric disorder, an affective disorder, depression, hypomania, panic attacks, anxiety, excessive elation, bipolar depression, bipolar disorder (manic-depression), seasonal mood (or affective) disorder, schizophrenia and other psychoses, lissencephaly syndrome, anxiety syndromes, anxiety disorders, phobias, stress and related syndromes, cognitive function disorders, aggression, drug and alcohol abuse, obsessive compulsive behavior syndromes, borderline personality disorder, non-senile dementia, post-pain depression, post-partum depression, cerebral palsy, and combinations thereof; or
wherein said nervous system disorder related to cellular trauma and/or injury is selected from neurological traumas and injuries, surgery related trauma and/or injury, retinal injury and trauma, injury related to epilepsy, spinal cord injury, brain injury, brain surgery, trauma related brain injury, trauma related to spinal cord injury, brain injury related to cancer treatment, spinal cord injury related to cancer treatment, brain injury related to infection, brain injury related to inflammation, spinal cord injury related to infection, spinal cord injury related to inflammation, brain injury related to environmental toxin, spinal cord injury related to environmental toxin, and combinations thereof; or
wherein said neurologically related condition is selected from learning disorders, memory disorders, autism, attention deficit disorders, narcolepsy, sleep disorders, cognitive disorders, epilepsy, temporal lobe epilepsy, and combinations thereof.

13. A method of treating a mood disorder in a subject or patient, said method comprising
administering an HDac inhibitory agent, optionally in combination with another an HDac inhibitory agent and/or another neurogenic agent, to a subject or patient that is
a) under treatment with anti-cancer chemotherapy and/or radiation therapy, or
b) diagnosed as having epilepsy or having seizures associated with epilepsy,
to produce an improvement in said mood disorder.

14. The method of claim 13, wherein said mood disorder is selected from depression, anxiety, hypomania, panic attacks, excessive elation, seasonal mood (or affective) disorder, schizophrenia and other psychoses, lissencephaly syndrome, anxiety syndromes, anxiety disorders, phobias, stress and related syndromes, aggression, non-senile dementia, post-pain depression, and combinations thereof.

15. The method of claim 13, wherein said HDac inhibitory agent is valproic acid.

16. A method of protecting neural cells from damage or toxicity, said method comprising
contacting a population of neural cells with an HDac inhibitory agent to protect said cells.

17. The method of claim 16, wherein the level of differentiation of said protected cells into astrocytes is limited or inhibited.

18. A method to maintain or reduce the differentiation of neural cells into astrocytes, said method comprising
contacting a population of neural cells with an HDac inhibitory agent to maintain or reduce their differentiation into cells of an astrocytic lineage.

19. The method of claim 18, wherein said cells are in a subject or patient with a nervous system disorder related to disease, cellular degeneration, a psychiatric condition, cellular trauma and/or injury, or another neurologically related condition.

20. A method to reduce or inhibit aberrant differentiation, proliferation and/or migration of neural cells in a tissue, said method comprising
administering an HDac inhibitory agent to a subject or patient to reduce or inhibit aberrant differentiation, proliferation and/or migration of neural cells into a tissue.

21. The method of claim 20, wherein said subject or patient has a nervous system disorder related to disease, cellular degeneration, a psychiatric condition, cellular trauma and/or injury, or another neurologically related condition.

22. The method of claim 16, wherein said cells are
in a human patient or in a tissue of a human patient; optionally diagnosed with cancer, or
in a human patient treated with chemotherapy and/or radiation; or
in a human patient diagnosed as having epilepsy or having seizures associated with epilepsy.

23. A method of preparing cells or tissue for transplantation to a subject or patient, said method comprising
contacting said cell or tissue with an HDac inhibitory agent, optionally in combination with another HDac inhibitory agent and/or another neurogenic agent, to stimulate or increase neurogenesis in said cell or tissue.

24. A method of maintaining, stabilizing, stimulating, or increasing neurodifferentiation in a cell or tissue, said method comprising
contacting said cell or tissue with an HDac inhibitory agent to maintain, stabilize stimulate, or increase neurodifferentiation in said cell or tissue.

25. The method of claim 24, further comprising contacting said cell or tissue with an additional neurogenic or neuroproliferative agent to produce neurogenesis in said cell or tissue, optionally wherein said cell or tissue exhibits decreased neurogenesis or is subjected to an agent or condition which decreases or inhibits neurogenesis; or
wherein said cell or tissue is in an animal subject or a human patient, optionally wherein said subject or patient is in need of neurogenesis or has been diagnosed with a disease, condition, or injury of the central or peripheral nervous system; or
wherein said cell or tissue exhibits aberrant neurogenesis or neuroproliferation.
